(19) United States
(12) Patent Application Publication
(10) Pub. No.: US 2026/0317810 A1
(43) Pub. Date: Oct. 15, 2026

(54) ARRAYED DETECTION OF TARGET MOLECULES VIA CRISPR CASCADE REACTIONS

(71) Applicant: VedaBio, Inc., San Diego, CA (US)

(72) Inventors: Jorge Bernate, San Diego, CA (US); Phillip Belgrader, San Diego, CA (US); Don Masquelier, San Diego, CA (US); Andrea Bryan, San Diego, CA (US); Ariana Mostafa, San Diego, CA (US); Jacob Berger, San Diego, CA (US); Ashish Pandey, San Diego, CA (US); Ketki Sawant, San Diego, CA (US); Ravi Vijaynendran, San Diego, CA (US); Anurup Ganguli, San Diego, CA (US)

(21) Appl. No.: 19/481,079

(22) PCT Filed: May 14, 2024
(86) PCT No.: PCT/US24/29305

§ 371 Date: Nov. 3, 2025

Related U.S. Application Data
(60) Provisional application No. 63/466,668, filed on May 15, 2023.

(51) Int. Cl. C12Q 1/6818; C12N 9/22; C12N 15/11; C12Q 1/6825; C12Q 1/689

(52) U.S. Cl. CPC C12Q 1/6818; C12N 9/226; C12N 15/11; C12Q 1/6825; C12Q 1/689; C12N 2310/20; C12N 2320/10; C12Q 2600/16

(57) ABSTRACT

The present disclosure relates to assay modules on which multiplex nucleic acid detection assays are performed to detect target nucleic acids of interest from several to many sources in a sample without amplification of the target nucleic acids of interest. The assay modules introduce a sample simultaneously to several to many different target-specific modalities.

### RELATED CASES

This application is a 35 U.S.C. § 371 filing of PCT/US24/29305, filed 14 May 2024, which claims priority to U.S. Ser. No. 63/466,668, filed 15 May 2023, both of which are incorporated by reference in their entirety.

### FIELD OF THE INVENTION

The present disclosure relates to assay modules on which multiplexed nucleic acid assays are performed to detect target nucleic acids of interest from several to many source organisms in a sample without amplification.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Rapid and accurate identification of, e.g., infectious agents, microbe contamination, variant nucleic acid sequences that indicate the present of diseases such as cancer, or contamination by heterologous sources is important in order to select correct treatment; identify tainted food, pharmaceuticals, cosmetics and other commercial goods; and to monitor the environment including identification of biothreats. Classic PCR and nucleic acid-guided nuclease or CRISPR (clustered regularly interspaced short palindromic repeats) detection methods rely on pre-amplification of target nucleic acids of interest to enhance detection sensitivity. However, amplification increases time to detection and may cause changes to the relative proportion of nucleic acids in samples that, in turn, lead to artifacts or inaccurate results. Improved technologies, including instrumentation, that allow very rapid and accurate detection of nucleic acids are therefore needed for timely diagnosis and treatment of disease, to identify toxins in consumables and the environment, as well as in other applications.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides assay modules upon which multiplexed signal boost assay methods are performed to detect target nucleic acids of interest in a sample without amplification of the target nucleic acids of interest. The “signal boost assays” or “signal boost nuclease assays” described herein comprise one or two different ribonucleoprotein (RNP) complexes and either blocked nucleic acid molecules, blocked primer molecules, or blocked guide nucleic acids, all of which allow for massive multiplexing without sacrificing sensitivity. The one-RNP embodiment comprises an RNP comprising a nucleic acid-guided nuclease and with gRNAs designed with complementarity to one or more target nucleic acids of interest, and in many embodiments there may be several to many to a multiplexed number of different Cas12-RNPs specific for each target of interest.

In the two-RNP embodiment, the blocked nucleic acid molecules, blocked primer molecules, or blocked guide nucleic acids in the signal boost assay keep second ribonucleoprotein complexes (RNP2s) “locked” unless and until a target nucleic acid of interest activates the first ribonucleoprotein complexes (RNP1s). The present signal boost assays can detect target nucleic acids of interest (e.g., DNA, RNA and/or cDNA) in less than ten minutes in some embodiments (including sample prep) without the need for amplifying the target nucleic acid(s) of interest, thereby avoiding the drawbacks of multiplex DNA amplification, such as primer-dimerization. A particularly advantageous feature of the signal boost assay is that, with the exception of the gRNAs in RNP1, the signal boost assay components may be the same in each assay no matter what target nucleic acids of interest are being detected; moreover, the gRNAs in the RNP1 are easily reprogrammed using traditional guide design methods. (See, e.g., U.S. Pat. Nos. 11,639,520; 11,702,686; 11,821,025; 11,970,730; 11,884,921; 11,820,983).

The assay modules described herein allow for performing the signal boost assays in an automated manner and in a short period of time. The assay modules employ two different approaches to introducing a sample to the assay components (e.g., RNP1, RNP2, blocked nucleic acids and reporter moieties); namely, 2D or parallel modules and 3D or array modules. Either assay module allows for several to many different target nucleic acids of interest to be detected simultaneously in a short period of time. Moreover, the assay modules may be incorporated into an assay instrumentation system which comprises a sample prep module and means for detection of the reporter moieties.

Thus, there is provided in one embodiment an assay module for identifying one or more target nucleic acids of interest from two or more source organisms in a sample comprising: at least one inlet coupled to a sample splitting region, wherein the sample splitting region is fluidically coupled to a plurality of first fluid channels and each first fluid channel is fluidically coupled to a first partition; a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, and wherein different RNP1s reside in different first partitions; second ribonucleoprotein complexes (RNP2s) comprising a second nucleic acid-guided nuclease and a second guide nucleic acid complementary to a portion of blocked nucleic acid molecules; the blocked nucleic acid molecules; reporter moieties; a pump configured to provide negative or positive pressure to the assay module; and a detection and imaging zone.

In some aspects, the RNP1s are lyophilized and in some aspects, the RNP1s are air dried.

In some aspects, the reporter moieties and blocked nucleic acid molecules are introduced in the sample splitting region with the sample and the RNP2s reside in the first partitions with the RNP1s, in yet other aspects, the RNP2s, reporter moieties and blocked nucleic acid molecules are introduced in the sample splitting region with the sample.

In some aspects, there are at least ten first fluid channels and ten first partitions, or at least twenty first fluid channels and twenty first partitions, or at least twenty-five first fluid channels and twenty-five first partitions, or at least thirty first fluid channels and thirty first partitions, or at least forty first fluid channels and forty first partitions, or at least fifty first fluid channels and fifty first partitions.

In some aspects, there are valves in the first fluid channels between the sample splitting region and the first partitions.

Some aspects include second fluid channels connecting the first partitions to second partitions, and wherein the RNP2s, blocked nucleic acid molecules, and reporter moieties reside in the second partitions.

In some aspects, there are at least ten first and second fluid channels and ten first and second partitions, or at least twenty first and second fluid channels and twenty first and second partitions, or at least twenty-five first and second fluid channels and twenty-five first and second partitions, or at least thirty first and second fluid channels and thirty first and second partitions, or at least forty first and second fluid channels and forth first and second partitions, or at least fifty first and second fluid channels and fifty first and second partitions

In some aspects, the assay module further comprises third fluid channels connecting the second partitions to third partitions and detection and imaging is performed in the third partitions.

In some aspects, the first partitions and/or the second partitions provide mixing of the sample and the RNP1s, RNP2s, blocked nucleic acid molecules and/or reporter moieties via bubbling, ultrasonic perturbation, magnetic beads or push/pull pressure changes.

In some aspects, there is provided a method for performing an assay cascade on a sample comprising the steps of: providing the assay module described above, wherein the detection and imaging zones are in each of the first partitions; providing the sample, RNP2s, blocked nucleic acid molecules, and reporter moieties to the sample splitting region; using the pump to flow the sample, RNP2s, blocked nucleic acid molecules, and reporter moieties from the sample splitting region through the first fluid channels into the first partitions; and detecting and imaging signals from the reporter moieties in the first partitions.

In yet another embodiment there is provided an assay module for identifying one or more target nucleic acids of interest from two or more source organisms in a sample comprising:

a bulk sample introduction region comprising at least one inlet and a fluid channel fluidically coupled sequentially to a plurality of first partitions; a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, wherein different RNP1s reside in different partitions, wherein the different RNP1s are coupled to the partitions or to supports within the partitions, and wherein reacted reporter moieties, if present, can be detected and imaged in each of the partitions; second ribonucleoprotein complexes (RNP2s) comprising a second nucleic acid-guided nuclease and a second guide nucleic acid complementary to a portion of blocked nucleic acid molecules; the blocked nucleic acid molecules; reporter moieties; and a pump configured to provide negative or positive pressure to the assay module.

In some aspects, wherein the RNP1s are lyophilized, and in other aspects, the RNP1s are air dried.

In some aspects of this embodiment, wherein the reporter moieties and blocked nucleic acid molecules are introduced in the bulk sample introduction region with the sample and the RNP2s are coupled to the first partitions or to supports within the partitions, in other embodiments, the RNP2s, reporter moieties and blocked nucleic acid molecules are introduced in the bulk sample introduction region with the sample.

In some aspects, the partitions provide mixing of the sample and the RNP1s, RNP2s, blocked nucleic acid molecules and/or reporter moieties via bubbling, ultrasonic perturbation, magnetic beads or push/pull pressure changes.

In some aspects of this embodiment, there are at least ten partitions, or at least twenty partitions, or at least twenty-five partitions, or at least thirty partitions, or at least forty partitions, or at least fifty first partitions.

In some aspects, there is provided a method for performing an assay cascade on a sample comprising the steps of: providing the bulk delivery assay module described above; providing the sample, RNP2s, blocked nucleic acid molecules, and reporter moieties to the bulk sample introduction region; using the pump to flow the sample, RNP2s, blocked nucleic acid molecules, and reporter moieties from bulk sample introduction region through the fluid channel into each of the partitions; and detecting and imaging signals from the reporter moieties in the partitions. In some aspects of this method, the second providing step is performed at below room temperature.

In a third embodiment there is provided an assay module for identifying one or more target nucleic acids of interest from two or more source organisms in a sample using reaction components comprising: a bulk sample introduction region comprising at least one inlet and a fluid channel fluidically coupled to a plurality of first partitions; a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, wherein different RNP1s reside in different first partitions, and wherein the different RNP1s are coupled to the first partitions or to supports within the first partitions; a plurality of second ribonucleoprotein complexes (RNP2s) comprising a second nucleic acid-guided nuclease and a second guide nucleic acid complementary to a portion of blocked nucleic acid molecules wherein the RNP2s reside in a plurality of second partitions fluidically coupled to the plurality of first partitions but separated from the plurality of first partitions by a frangible membrane, wherein the frangible membrane is ruptured at pressure X; blocked nucleic acid molecules and reporter moieties; a pump configured to provide negative or positive pressure to the assay module; and detection and imaging zones adjacent to the second partitions.

In some aspects, the RNP1s and RNP2s are lyophilized, and in other aspects, the RNP1s and RNP2s are air dried.

In some aspects, the reporter moieties and blocked nucleic acid molecules are introduced in the bulk sample introduction region with the sample, in other aspects, the reporter moieties and blocked nucleic acid molecules reside in the second partitions with the RNP2s, and in yet other aspects, the reporter moieties and blocked nucleic acid molecules reside in the first partitions with the RNP1s.

In some aspects, there is provided a method for performing an assay cascade on a sample comprising the steps of: providing the bulk delivery assay module describe above; providing the sample, blocked nucleic acid molecules, and reporter moieties to the bulk sample introduction region; using the pump at a pressure less than X to flow the sample, blocked nucleic acid molecules, and reporter moieties from the bulk sample introduction region through the fluid channel into each of the first partitions; allowing the sample to react with the RNP1s; using the pump to provide a pressure X to rupture the frangible membrane between the first and second partitions; allowing the unblocked nucleic acid molecules, if present, to react with the RNP2s in the second partitions; and detecting and imaging signals from the reporter moieties in the second partitions.

These aspects and other features and advantages of the invention are described below in more detail.

### BRIEF DESCRIPTION OF THE DRAWINGS

The foregoing and other features and advantages of the present invention will be more fully understood from the following detailed description of exemplary embodiments taken in conjunction with the accompanying drawings in which:

FIG. 1A is a schematic overview of three exemplary embodiments of methods for performing the signal boost assay on a 2D or parallel module.

FIG. 1B is a schematic overview of three exemplary embodiments of methods for performing the signal boost assay on a 3D or array module.

FIG. 1C shows an exemplary embodiment of a 2D or parallel module and a 3D or array module.

FIG. 1D is an overview of the general principles underlying the nucleic acid-guided nuclease signal boost assay described in detail herein, where target nucleic acids of interest from a sample do not need to be amplified before detection.

FIG. 2A is a diagram showing the sequence of steps in an exemplary signal boost assay utilizing blocked nucleic acid molecules.

FIG. 2B is a simplified graphic showing an exemplary blocked nucleic acid molecule and a method for unblocking the blocked nucleic acid molecules of the disclosure.

FIG. 3A is a diagram showing the sequence of steps in an exemplary signal boost assay utilizing circular blocked primer molecules and linear template molecules.

FIG. 3B is a diagram showing the sequence of steps in an exemplary signal boost assay utilizing circular blocked primer molecules and circular template molecules.

FIG. 4 is a diagram showing the sequence of steps in an exemplary signal boost assay utilizing blocked guide nucleic acid (gRNA) molecules.

FIG. 5 illustrates three exemplary embodiments of reporter moieties.

FIG. 6 is a graphic illustration of an exemplary workflow for the sample splitting and 3D or array modules.

FIG. 7A is a simplified graphic of an architecture and readout for an exemplary 2D or parallel module.

FIG. 7B is an illustration of an exemplary 2D or parallel module.

FIG. 7C is a diagram of a dead-ended channel design which may enhance even splitting of fluid across a channel network, such as that shown in FIG. 7B.

FIGS. 7D-7L are illustrations of perspectives of additional exemplary 2D or parallel modules.

FIG. 7M is an overview of the principles behind one sample splitting method of the present disclosure.

FIG. 8A is a simplified graphic of an architecture and readout for an exemplary a 3D or array module.

FIGS. 8B-8J are illustrations of various perspectives of 3D or array modules.

FIG. 9 comprises two graphs of repeat experiments measuring RNP1 activity after 72 hours of air drying.

FIG. 10 is a graph showing assay activity using air dried RNP1s and RNP2s.

It should be understood that the drawings are not necessarily to scale, and that like reference numbers refer to like features.

### Definitions

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

All of the functionalities described in connection with one embodiment of the compositions and/or methods described herein are intended to be applicable to the additional embodiments of the compositions and/or methods except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Note that as used herein and in the appended claims, the singular forms “a,” “an,” and “the” include plural referents unless the context clearly dictates otherwise. Thus, for example, reference to “a cell” refers to one or more cells, and reference to “a system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA, as RNA, or a combination of DNA and RNA (e.g., a chimeric nucleic acid).

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both limits, ranges excluding either or both of those included limits are also included in the invention.

The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context.

As used herein, the terms “binding affinity” or “dissociation constant” or “K_{d}” refer to the tendency of a molecule to bind (covalently or non-covalently) to a different molecule. A high K_{d} (which in the context of the present disclosure refers to blocked nucleic acid molecules binding to RNP2, blocked primer molecules binding to template molecules or blocked guide molecules binding to the second nucleic acid nuclease and/or the RNP2 activating nucleic acids) indicates the presence of more unbound molecules, and a low K_{d} (which in the context of the present disclosure refers to blocked nucleic acid molecules binding to RNP2, blocked primer molecules binding to template molecules or blocked guide molecules binding to the second nucleic acid nuclease and/or the RNP2 activating nucleic acids) indicates the presence of more bound molecules. In the context of the present disclosure and the binding of blocked or unblocked nucleic acid molecules or blocked or unblocked primer molecules or blocked or unblocked guide molecules, low K_{d} values are in a range from about 100 fM to about 1 aM or lower (e.g., 100 zM) and high K_{d} values are in the range of 100 nM-100 μM (10 mM) and thus are about 10⁵- to 10¹⁰-fold or higher as compared to low K_{d} values.

As used herein, the terms “binding domain” or “binding site” refer to a region on a protein, DNA, or RNA, to which specific molecules and/or ions (ligands) may form a covalent or non-covalent bond. By way of example, a polynucleotide sequence present on a nucleic acid molecule (e.g., the primer binding domain on a template molecule) may serve as a binding domain for a different nucleic acid molecule (e.g., an unblocked primer nucleic acid molecule). Characteristics of binding sites are chemical specificity, a measure of the types of ligands that will bond, and affinity, which is a measure of the strength of the chemical bond.

As used herein, the terms “blocked guide molecule”, “blocked guide nucleic acid”, “blocked guide RNA” and “blocked gRNA” refer to CRISPR guide nucleic acids that cannot bind to the first or second RNP complex to activate cis- or trans-cleavage. The terms “unblocked guide molecule”, “unblocked guide nucleic acid”, “unblocked guide RNA” and “unblocked gRNA” refer to a formerly blocked gRNA that can bind to the second RNP complex (RNP2) to activate trans-cleavage of additional blocked gRNAs.

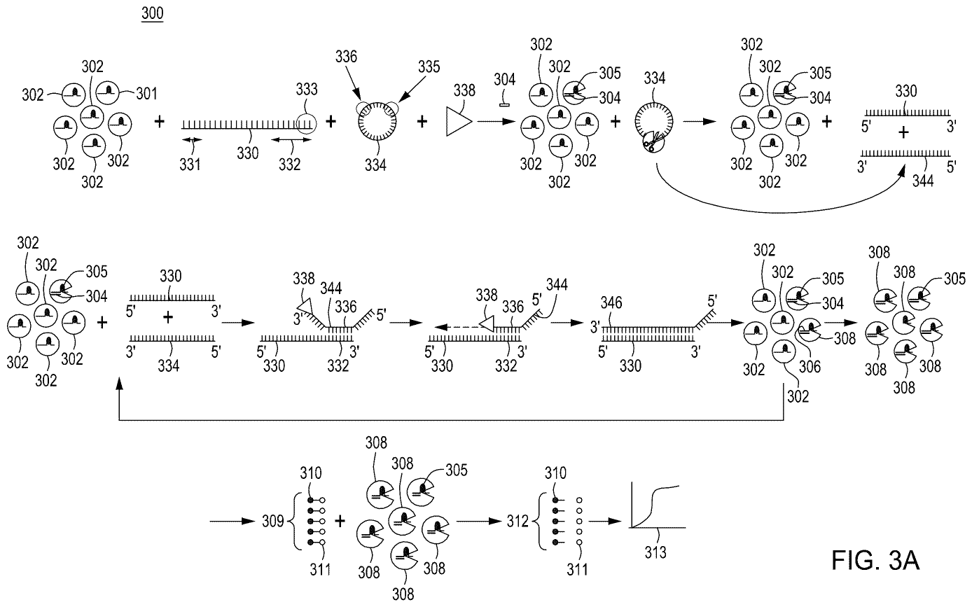

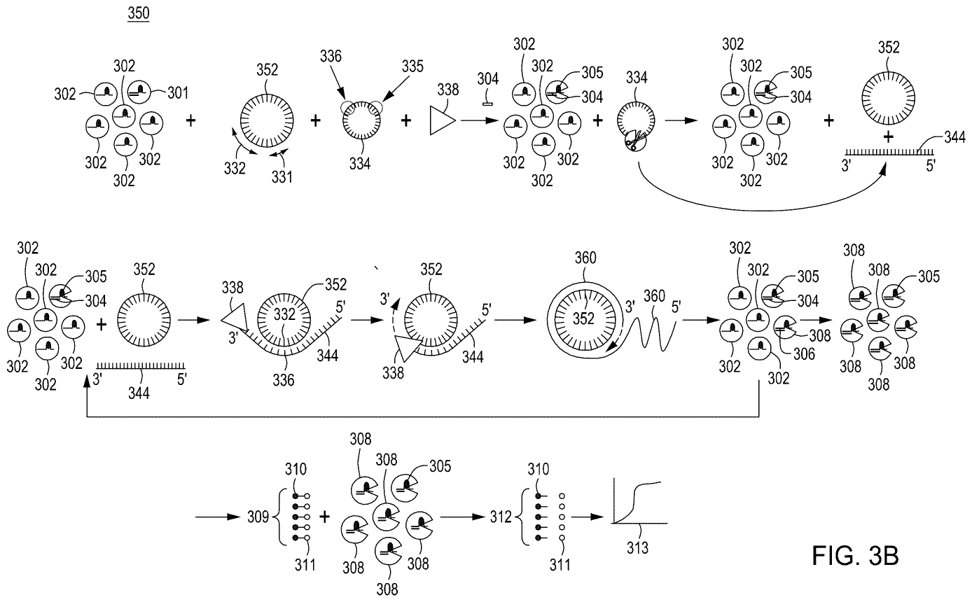

As used herein, the terms “blocked nucleic acid molecule” or “high K_{d} nucleic acid” refer to nucleic acid molecules that cannot bind to the first or second RNP complex to activate cis- or trans-cleavage. “Unblocked nucleic acid molecule” refers to a formerly blocked nucleic acid molecule that can bind to the second RNP complex (RNP2) to activate trans-cleavage of additional blocked nucleic acid molecules. A “blocked nucleic acid molecule” may be a “blocked primer molecule” in some embodiments of the signal boost assay as described below in relation to FIGS. 3A and 3B, and an “unblocked primer molecule” refers to a formerly blocked primer molecule that can bind to a template molecule and in the presence of a polymerase and dNTPs be extended to product a synthesized activating molecule.

The terms “Cas RNA-guided nucleic acid-guided nuclease” or “CRISPR nuclease” or “nucleic acid-guided nuclease” refer to a CRISPR-associated protein that is an RNA-guided nucleic acid-guided nuclease suitable for assembly with a sequence-specific gRNA to form a ribonucleoprotein (RNP) complex.

As used herein, the terms “cis-cleavage”, “cis-nucleic acid-guided nuclease activity”, “cis-mediated nucleic acid-guided nuclease activity”, “cis-nuclease activity”, “cis-mediated nuclease activity”, and variations thereof refer to sequence-specific cleavage of a target nucleic acid of interest, including an unblocked nucleic acid molecule, a synthesized activating molecule, or an RNP2 activating nucleic acid by a nucleic acid-guided nuclease in an RNP complex. Cis-cleavage is a single turn-over cleavage event in that only one substrate molecule is cleaved per event.

The term “complementary” as used herein refers to Watson-Crick base pairing between nucleotides and specifically refers to nucleotides hydrogen-bonded to one another with thymine or uracil residues bonded to adenine residues by two hydrogen bonds and cytosine and guanine residues bonded by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-ATCGAT-5′ is 100% complementary to a region of the nucleotide sequence 5′-GCTAGCTAG-3′.

As used herein, the term “contacting” refers to placement of two moieties in direct physical association, including in solid or liquid form. Contacting can occur in vitro with isolated cells (for example in a tissue culture dish or other vessel) or in samples or in vivo by administering an agent to a subject.

A “control” is a reference standard of a known value or range of values.

The terms “guide nucleic acid” or “guide RNA” or “gRNA” refer to a polynucleotide comprising 1) a crRNA region or guide sequence capable of hybridizing to the target strand of a target nucleic acid of interest, and 2) a scaffold sequence capable of interacting or complexing with a nucleic acid-guided nuclease. The crRNA region of the gRNA is a customizable component that enables specificity in every nucleic acid-guided nuclease reaction. A gRNA can include any polynucleotide sequence having sufficient complementarity with a target nucleic acid of interest to hybridize with the target nucleic acid of interest and to direct sequence-specific binding of a ribonucleoprotein (RNP) complex containing the gRNA and nucleic acid-guided nuclease to the target nucleic acid.

“Modified” refers to a changed state or structure of a molecule. Molecules may be modified in many ways including chemically, structurally, and functionally. In one embodiment, a nucleic acid molecule (for example, a blocked nucleic acid molecule) may be modified by the introduction of non-natural nucleosides, nucleotides, and/or internucleoside linkages. In another embodiment, a modified protein (e.g., a modified or variant nucleic acid-guided nuclease) may refer to any polypeptide sequence alteration which is different from the wildtype.

As used herein, a “partition” is an isolate region (e.g., a feature surrounded by an interstitial region) or an isolate depression (e.g., a well) on a substrate, or a droplet. Partitions are used, in relation to the present disclosure, to compartmentalize a plurality of ribonucleoprotein complexes (RNP1s) comprising different guide nucleic acids (gRNA1s) or guide nucleic acids for different source organisms and/or other assay components (e.g., into separate wells, features, or droplets).

The terms “percent sequence identity”, “percent identity”, or “sequence identity” refer to percent (%) sequence identity with respect to a reference polynucleotide or polypeptide sequence following alignment by standard techniques. Alignment for purposes of determining percent sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32 (5): 1792-1797 (2004)). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul, et al., J. Mol. Biol., 215:403-410 (1990)).

As used herein, the terms “preassembled ribonucleoprotein complex”, “ribonucleoprotein complex”, “RNP complex”, or “RNP” refer to a complex containing a guide RNA (gRNA) and a nucleic acid-guided nuclease, where the gRNA is integrated with the nucleic acid-guided nuclease. The gRNA, which includes a sequence complementary to a target nucleic acid of interest, guides the RNP to the target nucleic acid of interest and hybridizes to it. The hybridized target nucleic acid/gRNA units are cleaved by the nucleic acid-guided nuclease. In the signal boost assays described herein, a first ribonucleoprotein complex (RNP1) includes a first guide RNA (gRNA) specific to a target nucleic acid of interest, and a first nucleic acid-guided nuclease, such as, for example, cas12a or cas14a for a DNA target nucleic acid, or cas13a for an RNA target nucleic acid. A second ribonucleoprotein complex (RNP2) used for signal amplification includes a second guide RNA specific to an unblocked nucleic acid or synthesized activating molecule (or, in some embodiments, an RNP2 activating nucleic acid), and a second nucleic acid-guided nuclease, which may be different from or the same as the first nucleic acid-guided nuclease.

As used herein, the terms “protein” and “polypeptide” are used interchangeably. Proteins may or may not be made up entirely of amino acids.

As used herein, the term “sample” refers to tissues; cells or component parts; body fluids, including but not limited to peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. “Sample” may also refer to specimens or aliquots from food; agricultural products; pharmaceuticals; cosmetics; nutraceuticals; personal care products; environmental substances such as soil, water (from both natural and treatment sites), air, or sewer samples; industrial sites and products; and chemicals and compounds. A sample further may include a homogenate, lysate or extract. A sample further refers to a medium, such as a nutrient broth or gel, which may contain cellular components, such as proteins or nucleic acid molecules.

The terms “target nucleic acid of interest”, “target sequence”, “target nucleic acid molecule of interest”, “target molecule of interest”, “target nucleic acid”, or “target of interest” refer to any locus that is recognized by a gRNA sequence in vitro or in vivo. The “target strand” of a target nucleic acid of interest is the strand of the double-stranded target nucleic acid that is complementary to a gRNA. The spacer sequence of a gRNA may be 50%, 60%, 75%, 80%, 85%, 90%, 95%, 97.5%, 98%, 99% or more complementary to the target nucleic acid of interest. Optimal alignment can be determined with the use of any suitable algorithm for aligning sequences. Full complementarity is not necessarily required provided there is sufficient complementarity to cause hybridization and trans-cleavage activation of an RNP complex. A target nucleic acid of interest can include any polynucleotide, such as DNA (ssDNA or dsDNA) or RNA polynucleotides. A target nucleic acid of interest may be located in the nucleus or cytoplasm of a cell such as, for example, within an organelle of a eukaryotic cell, such as a mitochondrion or a chloroplast, or it can be exogenous to a host cell, such as a eukaryotic cell or a prokaryotic cell. The target nucleic acid of interest may be present in a sample, such as a biological or environmental sample, and it can be a viral nucleic acid molecule, a bacterial nucleic acid molecule, a fungal nucleic acid molecule, or a polynucleotide of another organism, such as a coding or a non-coding sequence, and it may include single-stranded or double-stranded DNA molecules, such as a cDNA or genomic DNA, or RNA molecules, such as mRNA, tRNA, and rRNA. The target nucleic acid of interest may be associated with a protospacer adjacent motif (PAM) sequence, which may include a 2-5 base pair sequence adjacent to the protospacer. In some embodiments 1, 2, 3, 4, 5, 6, 7, 8, 9, 10, or more target nucleic acids can be detected by the disclosed method.

As used herein, the terms “trans-cleavage”, “trans-nucleic acid-guided nuclease activity”, “trans-mediated nucleic acid-guided nuclease activity”, “trans-nuclease activity”, “trans-mediated nuclease activity” and variations thereof refer to indiscriminate, non-sequence-specific cleavage of a target nucleic acid molecule by a nucleic acid-guided nuclease (such as by a Cas12, Cas13, and Cas14) which is triggered by binding of N nucleotides of a target nucleic acid molecule to a gRNA and/or by cis-(sequence-specific) cleavage of a target nucleic acid molecule. Trans-cleavage is a “multiple turn-over” event, in that more than one substrate molecule is cleaved after initiation by a single turn-over cis-cleavage event.

Type V CRISPR/Cas nucleic acid-guided nucleases are a subtype of Class 2 CRISPR/Cas effector nucleases such as, but not limited to, engineered Cas12a, Cas12b, Cas12c, C2c4, C2c8, C2c5, C2c10, C2c9, CasX (Cas12e), CasY (Cas12d), Cas 13a nucleases or naturally-occurring proteins, such as a Cas12a isolated from, for example, Francisella tularensis subsp. novicida (Gene ID: 60806594), Candidatus Methanoplasma termitum (Gene ID: 24818655), Candidatus Methanomethylophilus alvus (Gene ID: 15139718), and [Eubacterium] eligens ATCC 27750 (Gene ID: 41356122), and an artificial polypeptide, such as a chimeric protein.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like.

### DETAILED DESCRIPTION

The present disclosure provides assay modules for performing multiplex signal boost assays that detect several to many target nucleic acids of interest simultaneously. First, neither sample preparation methods nor the signal boost assays require amplification of the target nucleic acids of interest yet the signal boost assays retain sensitivity. The signal boost assays allow for massive multiplexing, and provide low cost, minimum automated workflow and result in less than ten minutes. The signal boost assays described herein comprise first and second ribonucleoprotein complexes and blocked nucleic acid molecules, blocked primer molecules or blocked guide molecules. The blocked nucleic acid molecules, blocked primer molecules and blocked guide molecules keep the second ribonucleoprotein complexes (RNP2s) “locked” unless and until a target nucleic acid of interest activates the first ribonucleoprotein complexes (RNP1s). The methods comprise the steps of designing guide nucleic acids specific to one to several to many target nucleic acids of interest in one or more source organisms, synthesizing first ribonucleoprotein complexes (and/or other signal boost assay components) in partitions, providing signal boost assay components, combining the signal boost assay components and sample, and detecting a signal that is generated if a target nucleic acid of interest is present in the sample. The assay modules described herein allow for the signal boost assay methods to be automated to provide results in some embodiments in less than 10 minutes.

Early and accurate identification of, e.g., infectious agents, microbe contamination, variant nucleic acid sequences that indicate the presence of diseases such as cancer or contamination by heterologous sources is important in order to select correct treatment; identify tainted food, pharmaceuticals, cosmetics and other commercial goods; and to monitor the environment. Nucleic acid-guided nucleases, such as Type V nucleic acid-guided nucleases, can be utilized for the detection of target nucleic acids of interest associated with diseases, food contamination and environmental threats. However, currently available nucleic acid detection such as quantitative PCR (also known as real time PCR or qPCR) or CRISPR-based detection assays such as SHERLOCK™ and DETECTR™ rely on DNA amplification, which requires time and may lead to changes to the relative proportion of nucleic acids, particularly in multiplexed nucleic acid assays. The lack of rapidity for these detection assays is due to the fact that there is a significant lag phase early in the amplification process where fluorescence above background cannot be detected. With qPCR, for example, there is a lag until the cycle threshold or Ct value, which is the number of amplification cycles required for the fluorescent signal to exceed the background level of fluorescence, is achieved and can be quantified.

The present disclosure describes an assay module for performing a signal boost assay that can detect several to many multiplexed target nucleic acids of interest from one to many source organisms (e.g., DNA, RNA and/or cDNA) in a multiplexed manner in less than ten minutes without the need for amplifying the target nucleic acid(s) of interest, thereby avoiding the drawbacks of multiplex amplification, such as primer-dimerization. As described in detail below, the sample preparation techniques utilize assaying for multiple target nucleic acids of interest from one to many to a massively multiplexed number of source organisms. The signal boost assays utilize signal boost mechanisms comprising various components including nucleic acid-guided nucleases; guide RNAs (gRNAs) incorporated into ribonucleoprotein complexes (RNP complexes) (and in come embodiments, blocked guide molecules); blocked nucleic acid molecules or blocked primer molecules (or in the case of blocked guide molecules RNP2 activating nucleic acids), reporter moieties, and, in some embodiments, polymerases and template molecules. A particularly advantageous feature of the signal boost assay is that, with the exception of the gRNA in RNP1 (i.e., gRNA1), the signal boost assay components may be identical no matter what target nucleic acids of interest are being detected, and gRNA1 is easily programmable using known techniques and gRNA design tools known in the art.

The first two embodiments of the signal boost assay provide a reaction mix comprising: a first ribonucleoprotein complex (RNP1) comprising a first Cas enzyme that exhibits both cis- and trans-cleavage activity and several to many first gRNAs; a second ribonucleoprotein complex (RNP2) comprising a second Cas enzyme that also exhibits both cis- and trans-cleavage activity and a second gRNA; either blocked nucleic acid molecules or blocked primer molecules; and reporter moieties, which may be separate molecules from the blocked nucleic acid molecules or blocked primer molecules or the reporter moieties may be incorporated into and part of the blocked nucleic acid molecules or blocked primer molecules. RNP1 is not activated unless and until a target nucleic acid molecule is detected.

The third embodiment of the signal boost assay provides a reaction mix comprising: a first ribonucleoprotein complex (RNP1) comprising a first Cas enzyme that exhibits both cis- and trans-cleavage activity and several to many first gRNAs; a second Cas enzyme that also exhibits both cis- and trans-cleavage activity; blocked guide nucleic acids that, when unblocked, can form a second ribonucleotide complexes (RNP2s) with the second Cas enzyme; RNP2 activating nucleic acids; and reporter moieties. Like the first two embodiments, RNP1 is not activated unless and until a target nucleic acid molecule is detected.

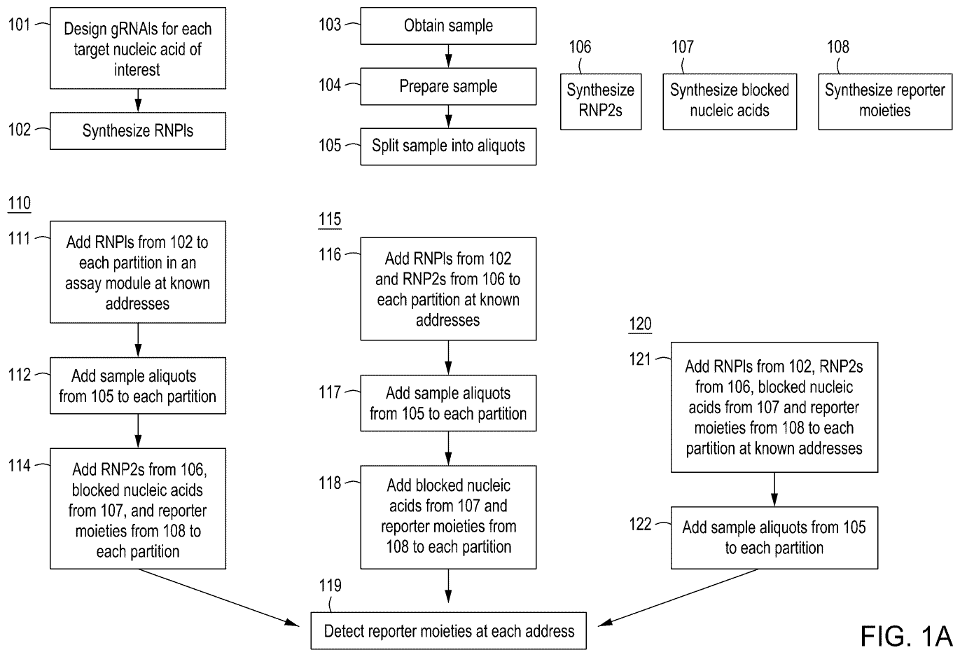

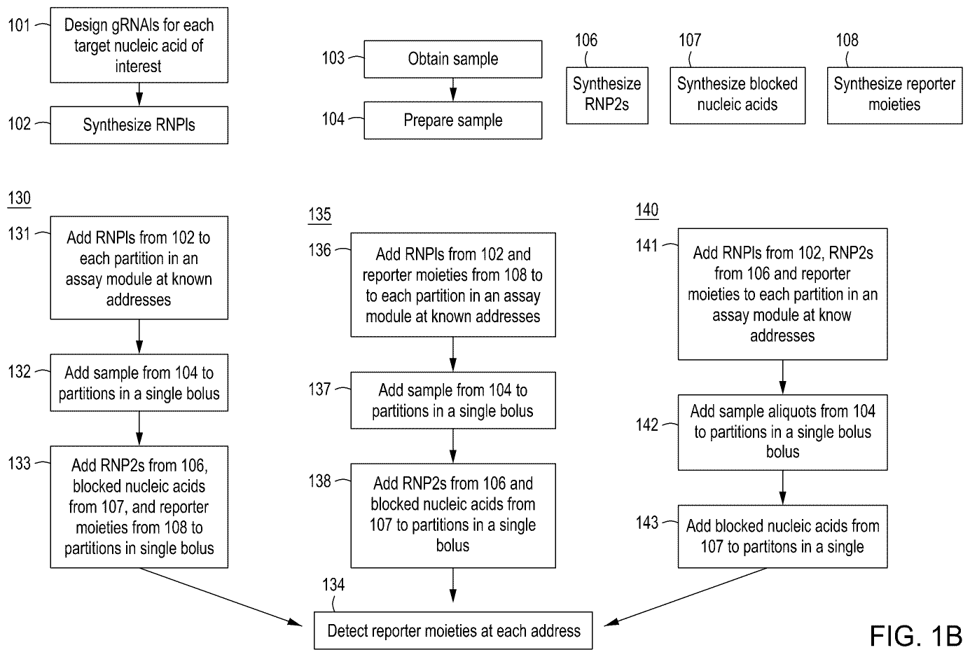

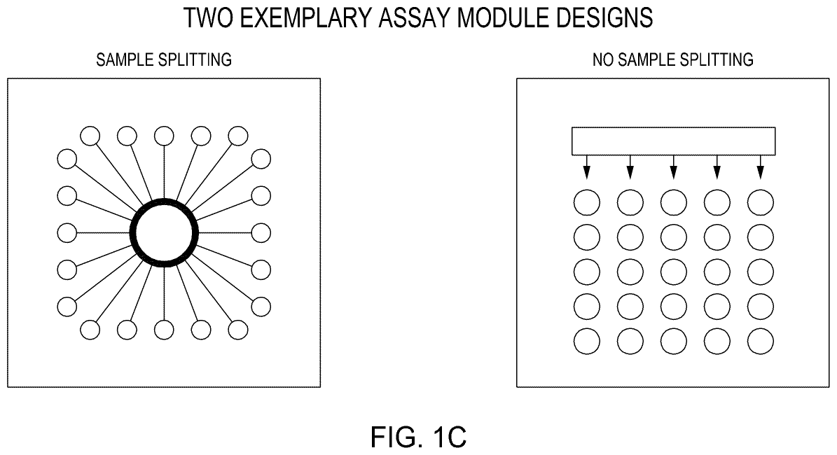

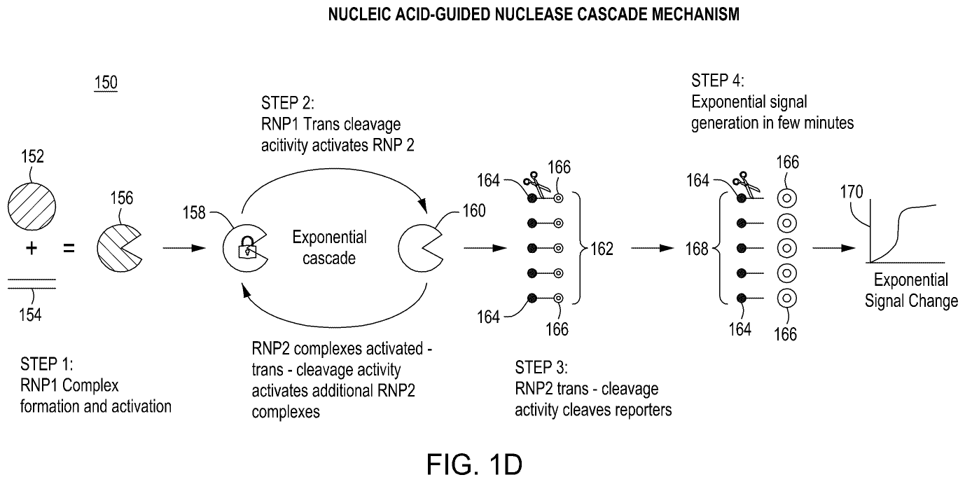

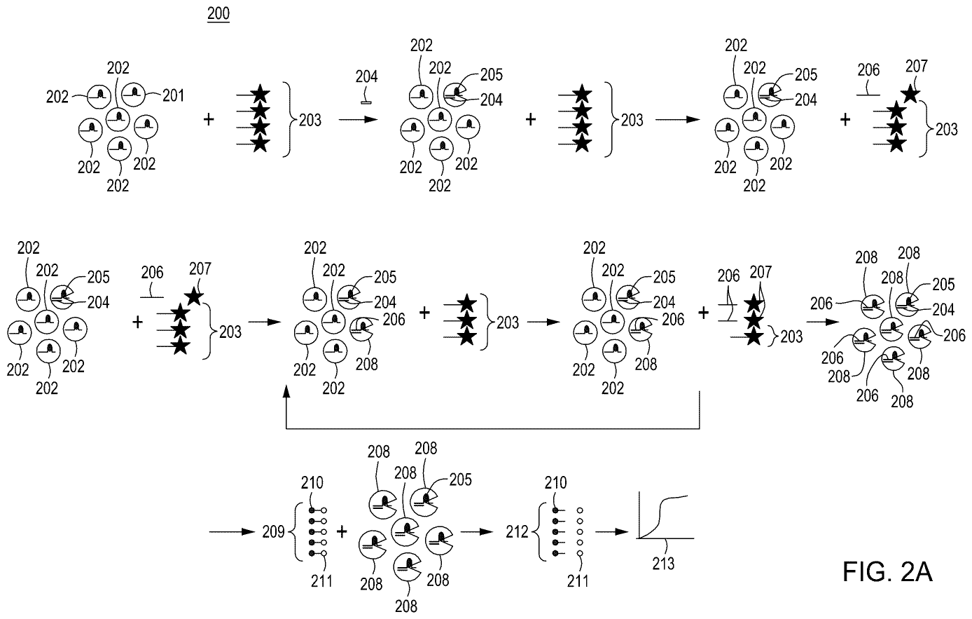

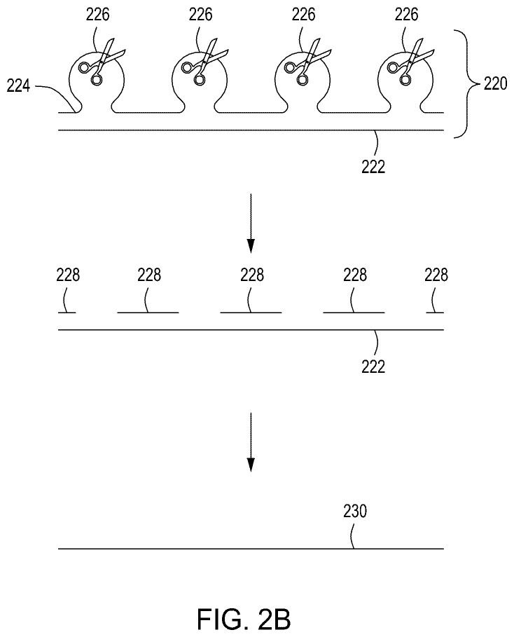

FIG. 1A is a schematic overview of three exemplary embodiments of methods for performing the signal boost assay on an assay module, where the methods are described in detail in relation to FIG. 2A. In a first step, first gRNAs (gRNA1s) are designed to several to many target nucleic acids of interest (101). That is, e.g., ten gRNA1s may be designed to detect ten different source organisms (e.g., bacteria and/or virus) or, e.g., two or more gRNA1s may be designed to target nucleic acids of interest in each of, e.g., five different source organisms. Designing the gRNA1s may be accomplished using known techniques and gRNA design tools known in the art. At step (102), RNP1s are synthesized with the gRNA1s and a first nucleic acid-guided nuclease of choice. Example II, infra, discloses a method for forming or synthesizing RNPs. Note that as described in this embodiment (110), the RNP1s were added to each partition in the assay module. As an alternative and preferably in some embodiments, the RNP1s can be formed in the partitions in the assay module. That is, the different gRNA1s can be distributed into partitions of known address, with the first nucleic acid guided nuclease added to each partition under conditions for forming the RNP1s. Separately, a sample is obtained (103). As described in detail below, a sample can be taken from any number of sources such as a biological sample including blood, serum, plasma, saliva, mucus, a nasal swab, a nasal pharyngeal swab, a buccal swab, a cell, a cell culture, and tissue from any mammal, such as, but not limited to, a human, primate, monkey, cat, dog, mouse, pig, cow, horse, sheep, and bat. Samples may also be obtained from any other source, such as air, water, soil, surfaces, food, beverages, nutraceuticals, clinical sites or products, industrial sites (including food processing sites) and products, plants and grains, cosmetics, personal care products, pharmaceuticals, medical devices, agricultural equipment and sites, and commercial products or processing facilities.

Once the sample is obtained, it is prepared (104) where source organisms present in the sample such as bacteria, viruses, fungi or other organisms or cells from organisms are lysed and the nucleic acids from these source organisms are fragmented into lengths of nucleic acids approximately 30 bp to 12,000 bp depending on the size of the genome. For example, it has been demonstrated that bead-beating E. coli cells for 1-3 minutes resulted in fragment sizes that look to be 500 bp-10 kb (see Proctor, et al., researchgate.net/publication/334045102, June 2019). It has also been demonstrated that heating E. coli DNA at 95° C. for 5 minutes results in fragment sizes of 500 bp-1000 bp (see Yang and Hang, J. of Biomolecular Techniques, 24:98-103 (2013). Human DNA fragments appear as a smear from 650 bp-12,000 bp when heated at 95° C. for 2 minutes, while 18 minutes of heating resulted in a smear ranging from 100 bp-500 bp. In addition to heating and bead beating, sonication may be used for DNA shearing; alternatively, enzymatic fragmentation may be employed. After nucleic acid fragmenting, the sample is split into a desired number of aliquots (105).

For embodiments utilizing blocked nucleic acid molecules (the blocked nucleic acid molecule embodiment illustrated in and described in relation to FIG. 2A will be used to describe the overviews in FIGS. 1A and 1B), second ribonucleoprotein complexes (RNP2s) comprising a second guide nucleic acid (i.e., gRNA2) and a second nucleic acid nuclease are synthesized. RNP2s are described in detail in relation to FIG. 2A. For most or all of the embodiments of the signal boost assay, the same RNP2 (gRNA2+second nucleic acid-guided nuclease) will be used in all partitions. The RNP2s are formed in the same manner as the RNP1s, and again, Example II, infra, discloses a method for forming or synthesizing RNPs.

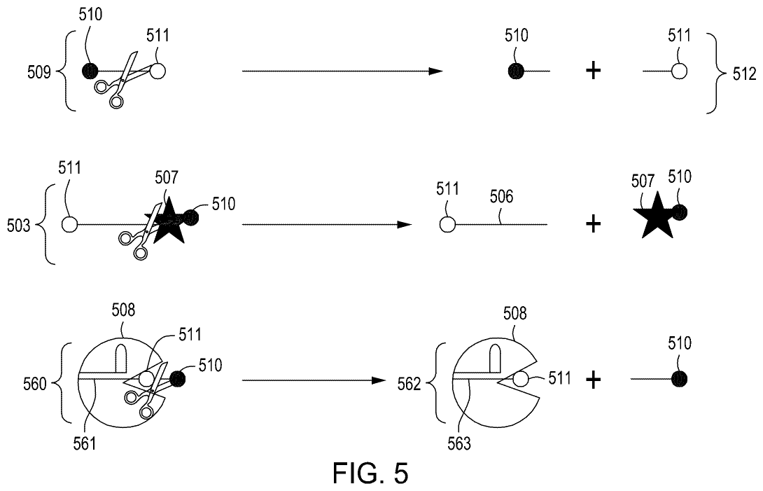

In addition to synthesis of the RNP1s and RNP2s, blocked nucleic acid molecules are synthesized (107). As described in detail below, blocked nucleic acid molecules keep the second ribonucleoprotein complexes (RNP2s) which boost the signal of a reporter “locked” unless and until a target nucleic acid of interest activates the RNP1s. Blocked nucleic acid molecules may be single-stranded or double-stranded molecules. If single-stranded, the blocked nucleic acid molecules comprise a first region recognized by the RNP2 complex (i.e., the target strand); one or more second regions not complementary to the first region forming at least one loop (which at least one of the at least one loops is a hairpin loop); and one or more third regions complementary to and hybridized to the first region forming at least one clamp. If double-stranded, the blocked nucleic acid molecules comprise a first strand recognized by the RNP2 complex (i.e., the target strand); and a second strand comprising one or more regions not complementary to the first strand forming at least one loop, as well as two or more regions complementary to and hybridized to the first strand forming at least two clamps. Finally, reporter moieties are synthesized (108). The reporter moieties produce a detectable signal upon induction, as described in detail below. In addition, FIG. 5 describes three different embodiments of reporter moieties of use in the signal boost assay.

In method (110), once the sample has been split into aliquots (105) and the signal boost assay components have been synthesized (102, 106, 107 and 108), the different gRNA1s are distributed into partitions or known address (111). Typically, one or more RNP1s designed to target source organism X (e.g., RNP1-1s) will be distributed in, e.g., partition 1; one or more RNP1s designed to target source organism Y (e.g., RNP1-2s) will be distributed in, e.g., partition 2; one or more RNP1s designed to target source organism Z (e.g., RNP1-3s) will be distributed in, e.g., partition 3, and so on. In other embodiments, RNP1s targeting two or more different target nucleic acids of interest may be distributed into a single partition, allowing for testing for two to many times the number source organisms; however, further testing would have to be done using the RNP1s from the single partition to identify the source organism(s) that were detected. The key is that the RNP1s that are in each partition at a particular address are known. The number of partitions will depend on the volume of the sample and the number of source organisms to be detected. In a typical diagnostic assay where the target nucleic acids of interest are at a low concentration (i.e., as low as attomolar range), the number of partitions will range from 2-100, or from 4-48 or from 4-24. However, in other applications of the signal boost assay, as many as 10,000 or more different nucleic acids may be detected in the signal boost assays.

Once the RNP1s have been distributed into partitions of known address (111), an aliquot of the sample is added to each partition (112). Once the sample aliquots have been distributed into each partition (112), the other signal boost assay components (RNP2s, blocked nucleic acid molecules and reporter moieties) are added to each partition (114) where reaction conditions are provided for the signal boost assay to take place. As discussed below, even at ambient temperatures of about 16-20° C. or less up to 48° C. the signal boost assay can detect target nucleic acids of interest from source organisms present in the sample. The source organisms are detected via activation of trans-cleavage activity of RNP1 by binding of a target nucleic acid molecule of interest to RNP1; trans-cleavage of the blocked nucleic acid molecules which then activates the trans-cleavage activity of RNP2; and trans-cleavage of more blocked nucleic acid molecules by RNP2 and trans-cleavage (induction) of reporter moieties. Thus, binding of a single target nucleic acid of interest to RNP1 initiates unblocking of the blocked nucleic acid molecules where the unblocked nucleic acid molecules bind to RNP2s initiating more unblocking of the blocked nucleic acid molecules and induction of reporter moieties in a continuing cascade, thereby “boosting” the signal produced by the reporter moieties.

In a final step, the signal generated by trans-cleavage of the reporter moieties is detected at each address (119). How the signals generated by the reporter moieties are detected will depend on the type of reporter moieties used; e.g., radioactive moieties, enzymes, prosthetic groups, fluorescent markers, luminescent markers, bioluminescent markers, metal particles, or protein-protein binding pairs as described below. In method (110) described above, the first ribonucleoproteins (RNP1s) comprising the first nucleic acid-guided nuclease and the first guide nucleic acids (gRNA1s) are partitioned and then the sample aliquots followed by the other assay components are added to each partition. As described below, many variations on the sequence of adding reagents are possible. Further, the various reagents may be added at different times but to the same partition(s), or the reagents may reside in separate partitions linked by fluidic channels. Detection, such as, e.g., fluorescent detection, may take place in a reaction well such as where the RNP2s and blocked nucleic acids reside, or the reaction may be moved to a specific well (smaller, larger, possessing specific properties) for detection.

In a different embodiment shown in FIG. 1A, method (115) begins by adding both the RNP1s and RNP2s into each partition at known addresses (116) (where, i.e., the RNP1s comprise different gRNA1s in each partition but the RNP2s in each partition are the same) before the sample aliquots (117) are added, followed by the addition of the other assay components (i.e., blocked nucleic acid molecules and reporter moieties) (118) and detection of signal from the reporter moieties (119). Method (115) thus accomplishes what method 110 does but alters the sequence of adding the sample aliquots and assay components. Similarly, method (120) is similar to methods (110) and (115); however, in method (120), all assay components (RNP1s from (102), RNP2s from (106), blocked nucleic acids from (107), and reporter moieties from (108)) are added to the partitions (with the different RNP1s at known addresses) (121) before the sample aliquots are added to each partition (122) followed by detection of signal from the reporter moieties (119). FIG. 1A thus illustrates three sequences for adding the sample aliquots and assay components to partitions comprising the RNP1s but other embodiments are possible, such as those described in relation to FIG. 1B.

In the methods (110, 115, and 120) just described, an alternative approach is that the gRNA1s may be in the partitions and the first nucleic acid-guided nuclease may be part of the reaction mix along with the other assay components. In this case, the RNP1s will form in the partitions (i.e., with the gRNA1s and the first nucleic acid-guided nuclease) in the presence of the other assay components. Although this alternative is possible, it is preferable that the RNP1s are pre-formed to speed reaction kinetics.

FIG. 1B is a schematic overview of three exemplary embodiments of methods for performing the signal boost assay on a 3D or array module. Instead of splitting the sample into aliquots and distributing the aliquots into the partitions comprising the RNP1s, the sample is delivered “in bulk” or as a “single bolus” to the partitions in the assay module. As with the methods shown in and described in relation to FIG. 1A, the signal boost assay embodiment described in detail in relation to FIG. 2A is described; however, the other signal boost assay embodiments, e.g., those employing either blocked primer molecules or blocked guide molecules, could be performed on the assay module although the assay components would be different as described below.

As with methods (110, 115 and 120) described in FIG. 1A, methods (130, 135, and 140) in FIG. 1B begin with a first step where gRNA1s are designed to several to many target nucleic acids of interest (101) by, e.g., using techniques and gRNA design tools known in the art. At step (102), RNP1s are synthesized with the gRNA1s and a first nucleic acid-guided nuclease of choice. In step (103), a sample is obtained and the prepared (104) where source organisms or cells present in the sample such as bacteria, viruses, fungi or other organisms are lysed and the nucleic acids fragmented. For embodiments utilizing blocked nucleic acid molecules, second ribonucleoprotein complexes (RNP2s) comprising a second guide nucleic acid (i.e., gRNA2) and a second nucleic acid nuclease are synthesized. As described above, for most or all of the embodiments of the signal boost assay, the same RNP2 (gRNA2+second nucleic acid-guided nuclease) will be used in all partitions. Unlike RNP1, RNP2 is not specific to a target nucleic acid of interest; instead, RNP2 is an assay component that boosts the signal from a reporter moiety when complexed with an unblocked nucleic acid molecule. In addition to synthesis of the RNP1s and RNP2s, blocked nucleic acid molecules are synthesized (107) as are reporter moieties (108).

In method (130), once the sample has been prepared (104) and the signal boost assay components have been synthesized (102, 106, 107 and 108), the different gRNA1s are distributed into partitions or known address (131). Typically, one or more RNP Is designed to target source organism X (e.g., RNP1-1s) will be distributed in, e.g., partition 1; one or more RNP1s designed to target source organism Y (e.g., RNP1-2s) will be distributed in, e.g., partition 2; one or more RNP1s designed to target source organism Z (e.g., RNP1-3s) will be distributed in, e.g., partition 3, and so on. Again, the key is that the RNP1s that are in each partition at a particular address are known. The number of partitions will depend on the volume of the sample and the number of source organisms to be detected. In a typical diagnostic assay where the target nucleic acids of interest are at a low concentration (i.e., as low as attomolar range), the number of partitions will range from 2-100 or from 2-50, or from 2-24. However, in other applications of the signal boost assay where samples are plentiful, as many as 10,000 or more different nucleic acids may be detected in the signal boost assays, where the RNP1s for detecting the different nucleic acids may reside in different partitions, or may be grouped into a smaller number of partitions to identify a target nucleic acid of interest as being one of a “group” of target nucleic acids, where identifying precisely which target nucleic acid is present will require testing against each target nucleic acid in the group separately.

Once the RNP1s have been distributed into partitions of known address (131), the sample is added to partition region of the array module in a single bolus (132) and allowed to contact the RNP1s for less than 5 minutes, or less than 2 minutes, or less than 1 minute, or less than 30 seconds to facilitate interaction of the nucleic acids (including the target nucleic acids of interest) in the sample and the RNP1s in the partitions. Once the sample has had time to interact with the RNP1s in the partitions (and, in some embodiments the excess sample is removed), the other signal boost assay components (RNP2s, blocked nucleic acid molecules and reporter moieties) are added to each partition in a single bolus (133) where reaction conditions are provided for the signal boost assay to take place. As discussed below, even at ambient temperatures of about 16-20° C. or less up to 48° C. the signal boost assay can detect source organisms present in the sample. In a final step, the signal generated by trans-cleavage of the reporter moieties is detected at each address (134).

In a different embodiment shown in FIG. 1B, method (135) begins by adding both the RNP1s and reporter moieties into each partition at known addresses (136) before the sample is added in a single bolus (137). Again, the RNP1s are allowed to interact with the nucleic acids (including the target nucleic acids of interest) in the sample, followed by the addition of the RNP2s and blocked nucleic acid molecules (138) in a single bolus, and detection of signal from the reporter moieties (134). Note again, method (135) accomplishes what method 130 does but alters the sequence of adding certain of the assay components and the sample. Note also that both methods (130, 135) deliver both the sample and certain of the assay components “in bulk” or in a single bolus. Similarly, method (140) is similar to methods (130) and (135); however, in method (140), all assay components aside from the RNP2s (i.e., RNP1s from (102), blocked nucleic acids from (107), and reporter moieties from 108) are added to the partitions (with the RNP1s at known addresses) (141) before the sample is added to the partitions (142) in the assay module in a single bolus. In method (140), RNP2 is added to the assay module last in a single bolus (143) followed by detection of signal from the reporter moieties (134). FIG. 1B thus illustrates three embodiments of adding the sample and assay components to partitions comprising the RNP1s all different from those illustrated in FIG. 1A. The three embodiments shown in FIG. 1B are applicable to the methods (110, 115, and 120) in FIG. 1A, and the three embodiments shown in FIG. 1A are applicable to the methods (130, 135 and 140) in FIG. 1B.

In the methods (130, 135, and 140) just described and with the methods (110, 115 and 120), an alternative approach is that the gRNA1s may be in the partitions and the first nucleic acid-guided nuclease may be part of the reaction mix of assay components along with the other assay components. In this case, the RNP1s will form in the partitions in the presence of the other assay components. Again, although this alternative is possible, it is preferable that the RNP1s are pre-formed to speed reaction kinetics.

One of ordinary skill in the art given the present disclosure will appreciate that the assay components may be combined in various configurations, where, e.g., there is a single RNP1 and RNP2 bead rather than two separate beads and comprising one or more of the reaction salts and buffers, and a blocked nucleic acid molecule and reporter moiety bead (which also may contain one or more of the reaction salts and buffers), and a “universal bead” comprising all of the assay components. Thus, the assay modules may comprise only one partition per channel. Again, the assay components may be added and distributed in many different configurations as long as different RNP1s are in different partitions.

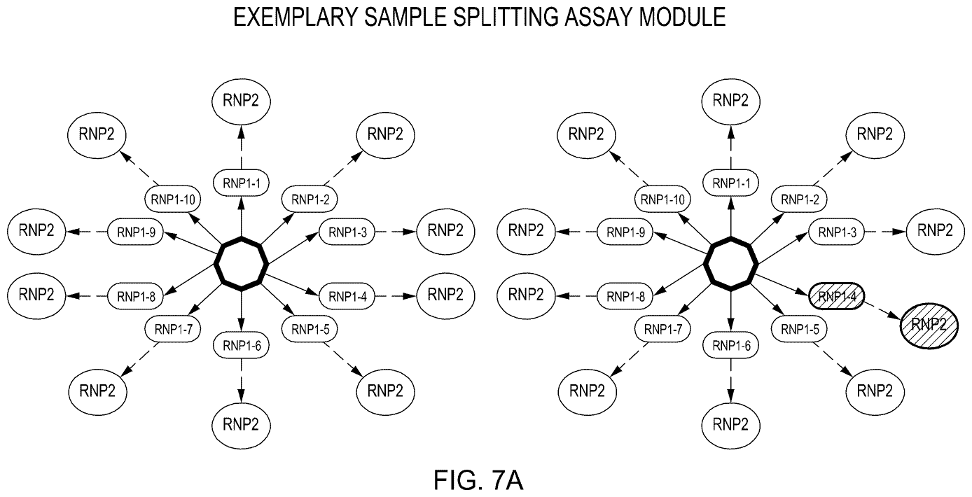

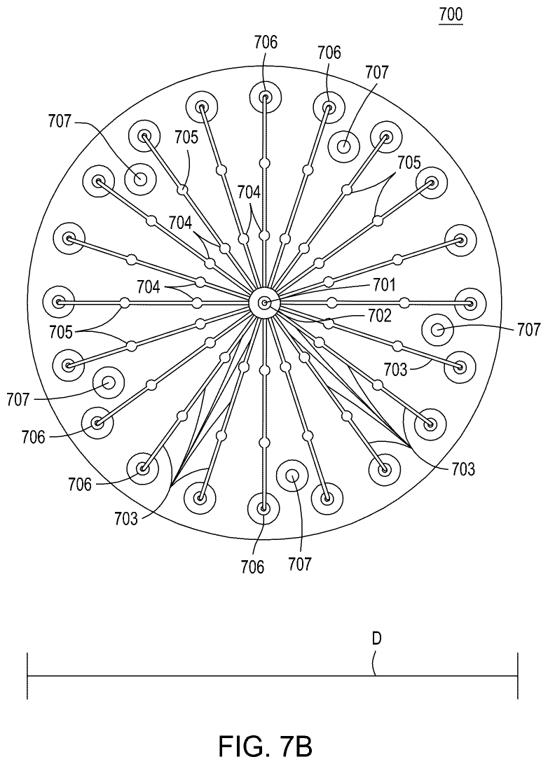

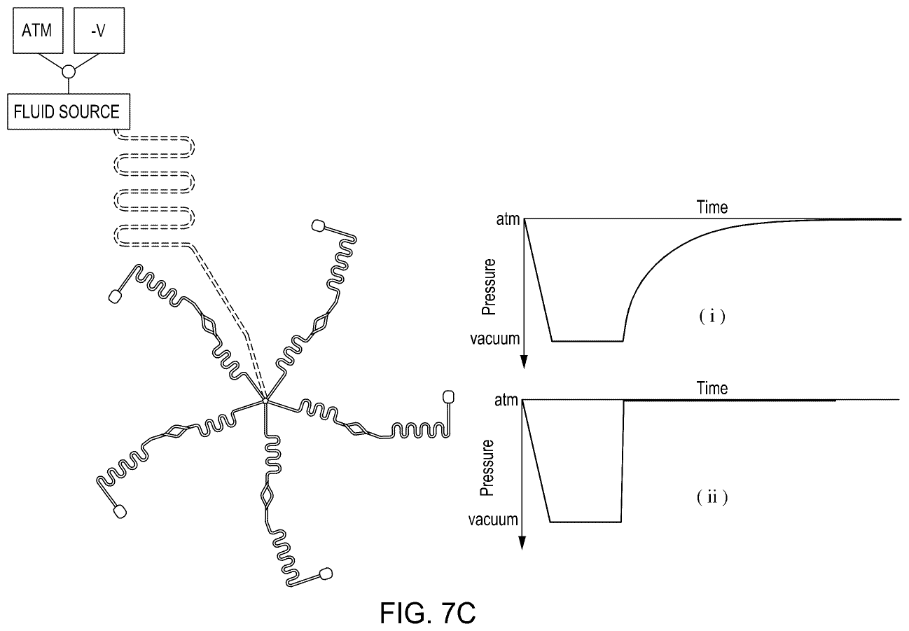

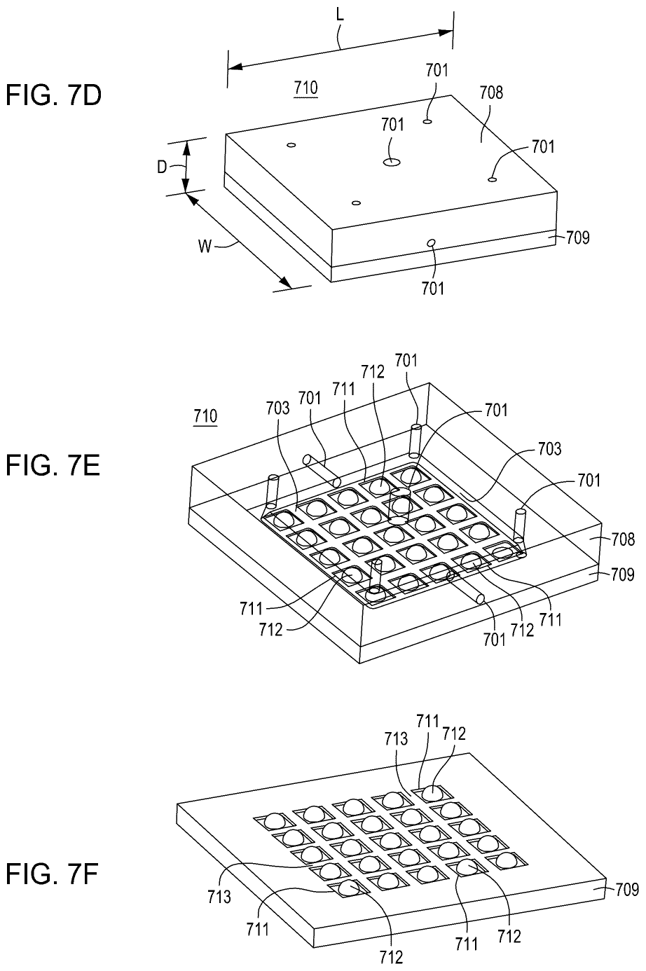

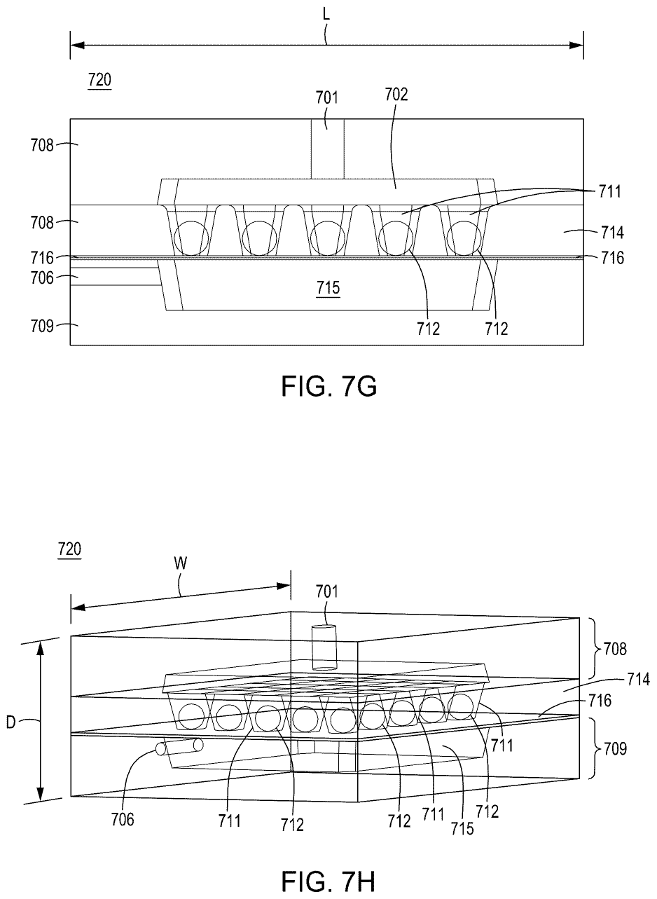

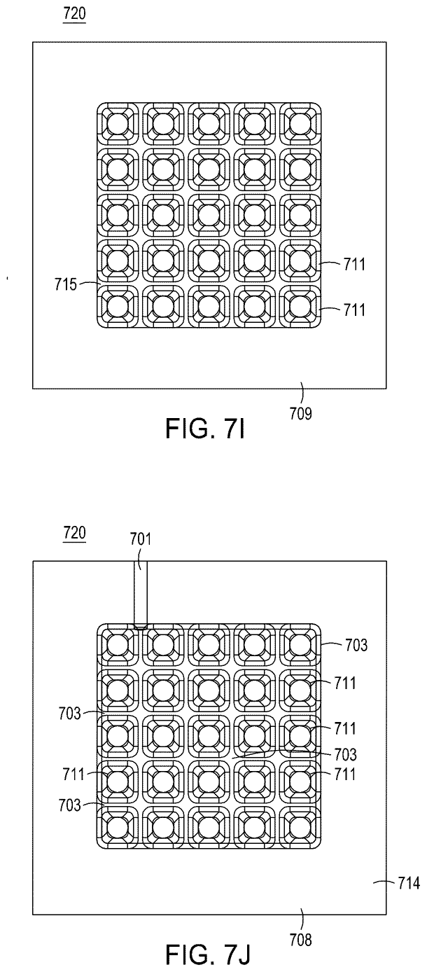

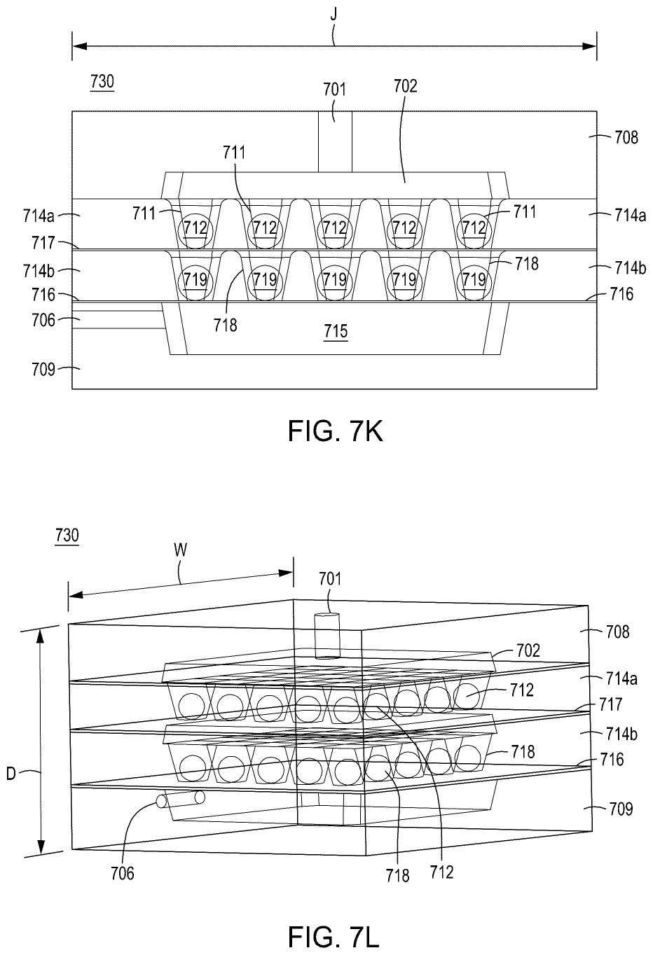

FIG. 1C shows a simplified exemplary embodiment of a 2D or parallel module and a 3D or array module. At left in FIG. 1C is an exemplary design of a 2D or parallel module where a central well or hub (“sample splitting zone”) where a sample can be distributed, with twenty channels radiating out from the central well, ending in terminal reaction wells. In this 2D or parallel module, a sample is distributed in the center of the assay module, then aliquots of the sample are driven by positive or negative pressure into the twenty channels and toward, e.g., the terminal reaction wells where the assay components are distributed. Each of the twenty terminal reaction wells comprises a known (and different) RNP1 and the other assay components (i.e., RNP2, blocked nucleic acids, and reporter moieties). FIGS. 7A-7C and the descriptions thereof show and describe modules in more detail.

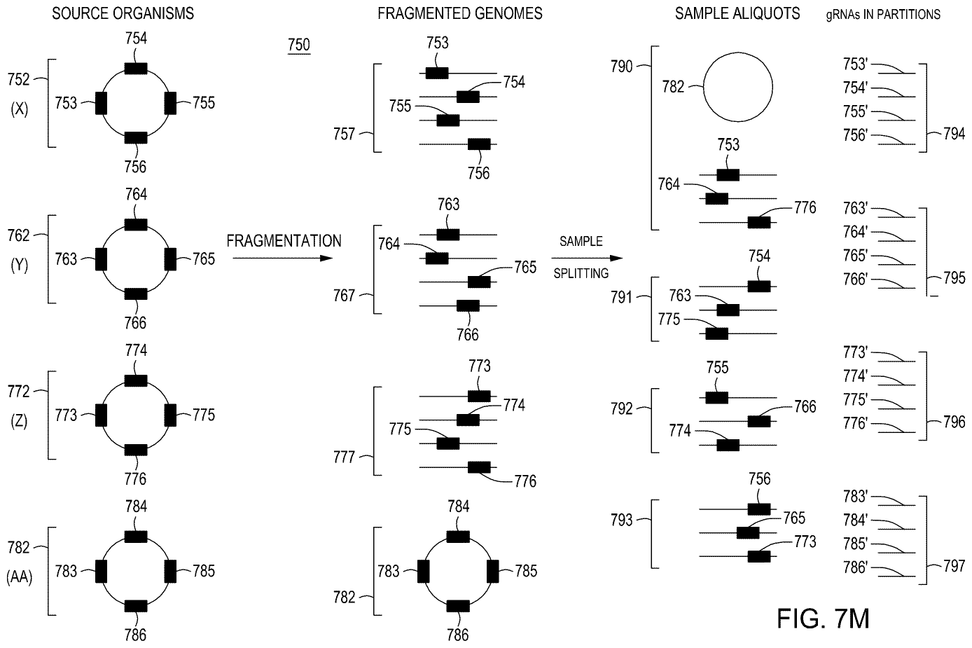

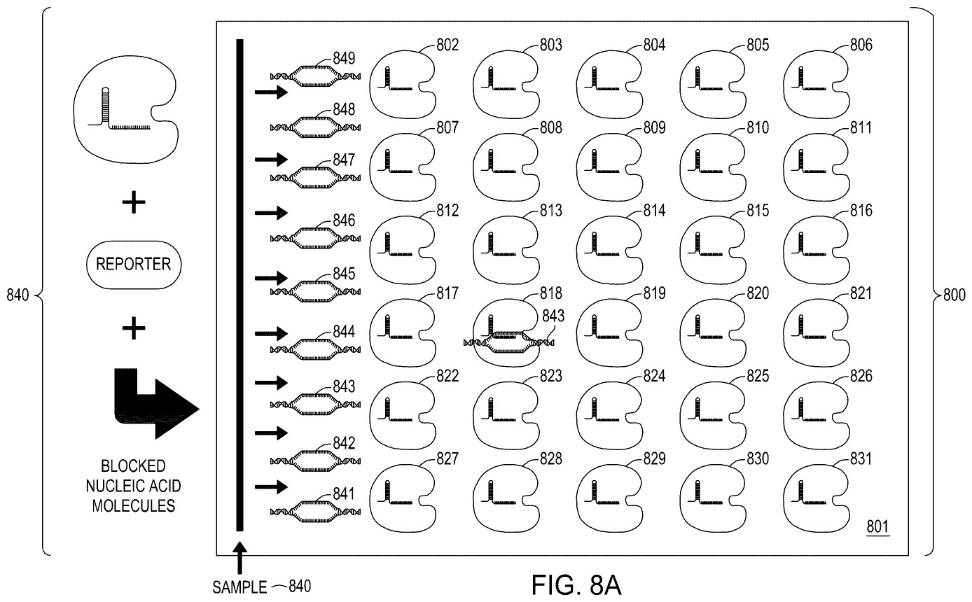

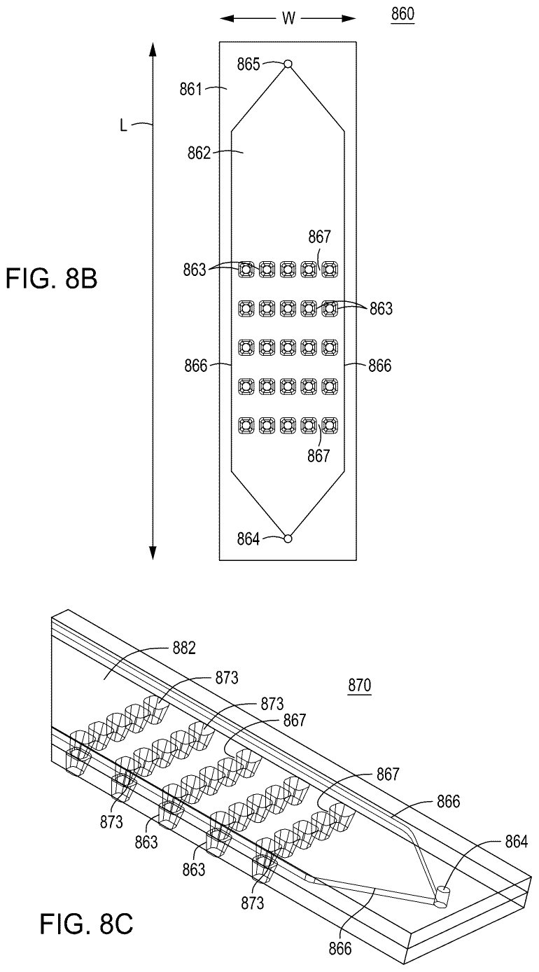

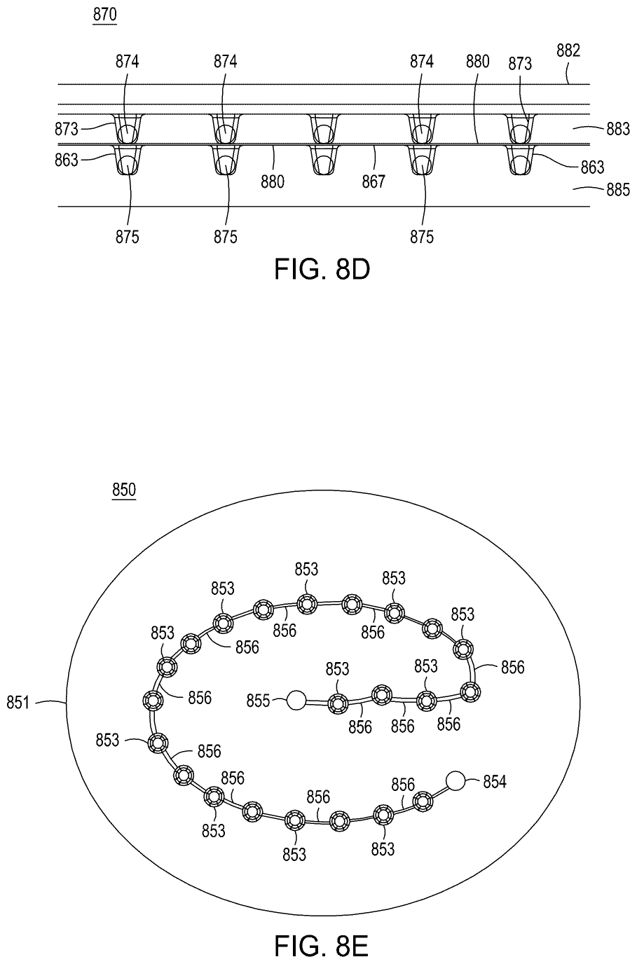

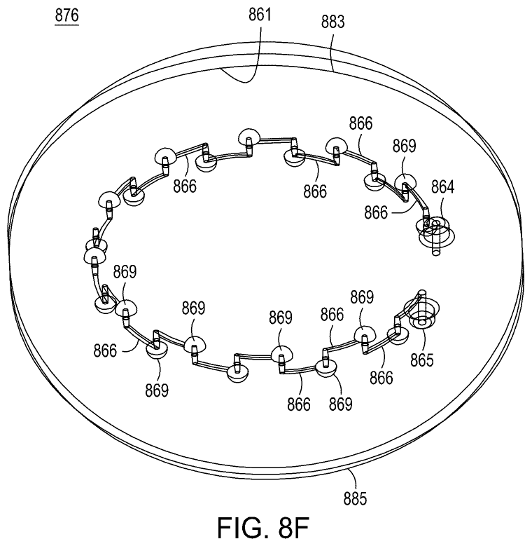

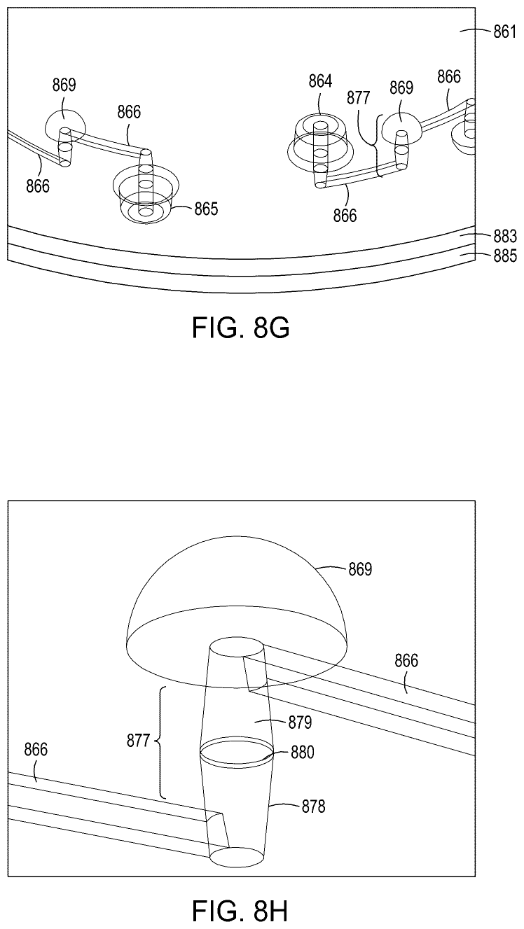

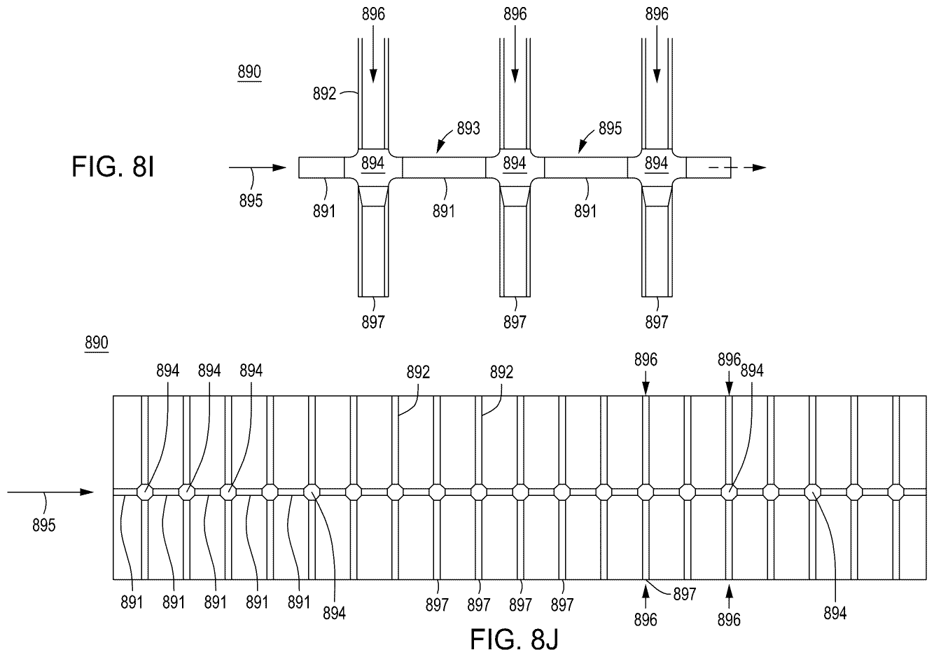

At right in FIG. 1C is an exemplary design of a 3D or array module comprising twenty-five reaction wells. In this array module, there is a 5×5 array of reaction wells each comprising a known (and different) RNP1 and the other assay components (i.e., RNP2, blocked nucleic acids, and reporter moieties). A sample is driven in a single bolus or in “bulk” by air displacement using positive or negative pressure across the reaction wells, where the sample is allowed to contact the RNP1s in the partitions to facilitate interaction of the nucleic acids (including the target nucleic acids of interest) in the sample and the RNP1s in the partitions. FIGS. 8A-8D and the descriptions thereof show and describe 3D or array modules in more detail.

FIG. 1D provides a simplified diagram demonstrating a method (150) of a signal boost assay. The signal boost assay is initiated when the target nucleic acid of interest (154) binds to and activates a first pre-assembled ribonucleoprotein complex (RNP1) (152). A ribonucleoprotein (RNP) complex comprises a guide RNA (gRNA) and a nucleic acid-guided nuclease, where the gRNA is integrated with the nucleic acid-guided nuclease. The gRNA, which includes a sequence complementary to the target nucleic acid of interest, guides an RNP complex to the target nucleic acid of interest and hybridizes to it. Typically, preassembled RNP complexes are employed in the reaction mix—as opposed to separate nucleic acid-guided nucleases and gRNAs—to facilitate rapid (and in the present signal boost assays, virtually instantaneous) detection of the target nucleic acid(s) of interest.

“Activation” of RNP1 refers to activating trans-cleavage activity of the nucleic acid-guided nuclease in RNP1 (156) by binding of the target nucleic acid of interest to the gRNA of RNP1. This binding initiates both trans-cleavage activity and cis-cleavage activity where the target nucleic acid of interest is cleaved by the nucleic acid-guided nuclease. This binding and/or cis-cleavage activity then initiates the trans-cleavage activity (i.e., multi-turnover activity) of the nucleic acid-guided nuclease, where trans-cleavage is indiscriminate, leading to non-sequence-specific cutting of nucleic acid molecules by the nucleic acid-guided nuclease of RNP1 (152). This trans-cleavage activity triggers activation of blocked ribonucleoprotein complexes (RNP2s) (158) in various ways, which are described in detail below. Each newly activated RNP2 (160) activates more RNP2s (158→160), which in turn cleave reporter moieties (162). The reporter moieties (162) may be a synthetic molecule linked or conjugated to a quencher (164) and a fluorophore (166) such as, for example, a probe with a dye label (e.g., FAM or FITC) on the 5′ end and a quencher on the 3′ end. The quencher (164) and fluorophore (166) can be about 20-30 bases apart (or about 10-11 nm apart) or less for effective quenching via fluorescence resonance energy transfer (FRET). Reporter moieties in various configurations also are described in greater detail in relation to FIG. 5 below.

As more RNP2s are unquenched (158→160), more trans-cleavage activity is activated and more reporter moieties are unquenched; thus, the binding of the target nucleic acid of interest (154) to RNP1 (152) initiates what becomes a cascade of signal production (170) from the reporter moieties, which increases exponentially; hence, the term “signal boost.” The signal boost assay thus comprises a single turnover event that triggers a multi-turnover event that then triggers another multi-turnover events in a “cascade.” As described below in relation to FIG. 5, the reporter moieties (162) may be provided as molecules that are separate from the other components of the nucleic acid-guided nuclease signal boost assay, or the reporter moieties may be covalently or non-covalently linked to the blocked nucleic acid molecules or synthesized activating molecules (i.e., the target molecules for the RNP2).

Various components of the sample prep methods, signal boost assay, and descriptions of how the signal boost assays work are described in detail below.

### Target Nucleic Acids of Interest

The target nucleic acids of interest may be a DNA, RNA, or cDNA molecule. Target nucleic acids of interest may be isolated from a sample by standard laboratory techniques. The target nucleic acids of interest originate from source organisms that are present in a sample, such as a biological sample from a subject (including non-human animals or plants), items of manufacture, or an environmental sample (e.g., water or soil). Non-limiting examples of biological samples include blood, serum, plasma, saliva, mucus, a nasal swab, a buccal swab, a cell, a cell culture, and tissue. The source of the sample could be any mammal, such as, but not limited to, a human, primate, monkey, cat, dog, mouse, pig, cow, horse, sheep, and bat. Samples may also be obtained from any other source, such as air, water, soil, surfaces, food, beverages, nutraceuticals, clinical sites or products, industrial sites (including food processing sites) and products, plants and grains, cosmetics, personal care products, pharmaceuticals, medical devices, agricultural equipment and sites, and commercial samples.

In some embodiments, the target nucleic acids of interest are from one to many infectious agents (e.g., a bacteria, protozoan, insect, worm, virus, or fungus) that affect mammals, including humans. As a non-limiting example, the target nucleic acids of interest could be one or more nucleic acid molecules from bacteria, such as Bordetella parapertussis, Bordetella pertussis, Chlamydia pneumoniae, Legionella pneumophila, Mycoplasma pneumoniae, Acinetobacter calcoaceticus-baumannii complex, Bacteroides fragilis, Enterobacter cloacae complex, Escherichia coli, Klebsiella aerogenes, Klebsiella oxytoca, Klebsiella pneumoniae group, Moraxella catarrhalis, Proteus spp., Salmonella enterica, Serratia marcescens, Haemophilus influenzae, Neisseria meningitidis, Pseudomonas aeruginosa, Stenotrophomonas maltophilia, Enterococcus faecalis, Enterococcus faecium, Listeria monocytogenes, Staphylococcus aureus, Staphylococcus epidermidis, Staphylococcus lugdunensis, Streptococcus agalactiae, Streptococcus pneumoniae, Streptococcus pyogenes, Chlamydia tracomatis, Neisseria gonorrhoeae, Syphilis (Treponema pallidum), Ureaplasma urealyticum, Mycoplasma genitalium, and/or Gardnerella vaginalis. Also, as a non-limiting example, the target nucleic acid of interest could be one or more nucleic acid molecules from a virus, such as adenovirus, coronavirus HKU1, coronavirus NL63, coronavirus 229E, coronavirus OC43, severe acute respiratory syndrome coronavirus 2 (SARS-COV-2), human metapneumovirus, human rhinovirus, enterovirus, influenza A, influenza A/H1, influenza A/H3, influenza A/H1-2009, influenza B, parainfluenza virus 1, parainfluenza virus 2, parainfluenza virus 3, parainfluenza virus 4, respiratory syncytial virus, herpes simplex virus 1, herpes simplex virus 2, human immunodeficiency virus (HIV), human papillomavirus, hepatitis A virus (HAV), hepatitis B virus (HBV), hepatitis C virus (HCV), and/or human parvovirus B19 (B19V). Also, as a non-limiting example, the target nucleic acids of interest could be one or more nucleic acid molecules from a fungus, such as Candida albicans, Candida auris, Candida glabrata, Candida krusei, Candida parapsilosis, Candida tropicalis, Cryptococcus neoformans, and/or Cryptococcus gattii. As another non-limiting example, the target nucleic acids of interest could be one or more nucleic acid molecules from a protozoan, such as Trichomonas vaginalis.

In some embodiments, other target nucleic acids of interest may be for non-infectious conditions, e.g., to be used for genotyping, including non-invasive prenatal diagnosis of, e.g., trisomies, other chromosomal abnormalities, and known genetic diseases such as Tay Sachs disease and sickle cell anemia. Other target nucleic acids of interest and samples include human biomarkers for cancer. Target nucleic acids of interest may include engineered biologics, including cells such as CAR-T cells, or target nucleic acids of interest from very small or rare samples, where only small volumes are available for testing.

The signal boost assays described herein are particularly well-suited for simultaneous testing of multiple to many targets via massively multiplexed gRNAs as described below. Pools of two to 10,000 target nucleic acid molecules of interest may be employed, e.g., pools of two to 1000, two to 100, two to 50, or two to 10 target nucleic acids of interest. As described above, the present disclosure contemplates two to several to many target nucleic acid molecules for loci from each source organism genome (or source chromosome or source cell or source tissue). If RNP1s from different source organisms are contained within the same partition, further testing may be used to identify the specific source organism, if desired.

The methods described herein do not require the target nucleic acids of interest to be DNA, and in fact it is specifically contemplated that the target nucleic acid of interest may be RNA.

### Nucleic Acid-Guided Nucleases

The signal boost assays comprise nucleic acid-guided nucleases in the reaction mix, either provided as a protein, a coding sequence for the protein, or, in many embodiments, in a ribonucleoprotein (RNP) complex. In some embodiments, the one or more nucleic acid-guided nucleases in the reaction mix may be, for example, a Cas nucleic acid-guided nuclease. Any nucleic acid-guided nuclease having both cis- and trans-cleavage activity may be employed, and the same nucleic acid-guided nuclease may be used for both RNP complexes or different nucleic acid-guided nucleases may be used in RNP1 and RNP2. For example, RNP1 and RNP2 may both comprise Cas12a nucleic acid-guided nucleases, or RNP1 may comprise a Cas13 nucleic acid-guided nuclease and RNP2 may comprise a Cas12a nucleic acid-guided nuclease or vice versa. Note that trans-cleavage activity is not triggered unless and until cis-cleavage activity (i.e., sequence specific activity) is initiated. Nucleic acid-guided nucleases include Type V and Type VI nucleic acid-guided nucleases, as well as nucleic acid-guided nucleases that comprise a RuvC nuclease domain or a RuvC-like nuclease domain but lack an HNH nuclease domain. Nucleic acid-guided nucleases with these properties are reviewed in Makarova and Koonin, Methods Mol. Biol., 1311:47-75 (2015) and Koonin, et al., Current Opinion in Microbiology, 37:67-78 (2020) and updated databases of nucleic acid-guided nucleases and nuclease systems that include newly-discovered systems include BioGRID ORCS (orcs: thebiogrid.org); GenomeCRISPR (genomecrispr.org); Plant Genome Editing Database (plantcrispr.org) and CRISPRCasFinder (crispercas.i2bc.paris-saclay.fr).

The type of nucleic acid-guided nuclease utilized in the method of detection depends on the type of target nucleic acid of interest to be detected. For example, a DNA nucleic acid-guided nuclease (e.g., a Cas12a, Cas14a, or Cas3) should be utilized if the target nucleic acid of interest is a DNA molecule, and an RNA nucleic acid-guided nuclease (e.g., Cas13a or Cas12g) should be utilized if the target nucleic acid of interest is an RNA molecule. Exemplary nucleic acid-guided nucleases include, but are not limited to, Cas RNA-guided DNA nucleic acid-guided nucleases, such as Cas3, Cas12a (e.g., AsCas12a, LbCas12a), Cas12b, Cas12c, Cas12d, Cas12e, Cas14, Cas12h, Cas12i, and Cas12j; Cas RNA-guided RNA nucleic acid-guided nucleases, such as Cas13a (LbaCas13, LbuCas13, LwaCas13), Cas13b (e.g., CccaCas13b, PsmCas13b), and Cas12g; and any other nucleic acid (DNA, RNA, or cDNA) targeting nucleic acid-guided nuclease with cis-cleavage activity and collateral trans-cleavage activity. In some embodiments, the nucleic acid-guided nuclease is a Type V CRISPR-Cas nuclease, such as Cas12a, Cas13a, or Cas14a. In some embodiments, the nucleic acid-guided nuclease is a Type I CRISPR-Cas nuclease, such as Cas3, and Type II and Type VI nucleic acid-guided nucleases may also be employed as long as the nucleic acid-guided nuclease exhibits trans-cleavage activity.

In an RNP with a single crRNA (i.e., lacking/without a traceRNA), Cas12a nucleases and related homologs and orthologs interact with a PAM (protospacer adjacent motif) sequence in a target nucleic acid for dsDNA unwinding and R-loop formation. Cas12a nucleases employ a multistep mechanism to ensure accurate recognition of spacer sequences in the target nucleic acid. The WED, REC1 and PAM-interacting (PI) domains of Cas12a nucleases are responsible for PAM recognition and for initiating invasion of the crRNA in the target dsDNA and for R-loop formation. It has been hypothesized that a conserved lysine residue is inserted into the dsDNA duplex, possibly initiating template strand/non-template strand unwinding. (See Jinek, et al, Mol. Cell, 73 (3): 589-600.e4 (2019).) PAM binding further introduces a kink in the target strand, which further contributes to local strand separation and facilitates base paring of the target strand to the seed segment of the crRNA while the displaced non-target strand is stabilized by interactions with the PAM-interacting domains. (Id.)

The nucleic acid-guided nucleases disclosed herein are wildtype or variants of wildtype Type V nucleases LbCas12a (Lachnospriaceae bacterium Cas12a), AsCas 12a (Acidaminococcus sp. BV3L6 Cas12a), CtCas12a (Candidatus Methanoplasma termitum Cas12a), EeCas12a (Eubacterium eligens Cas12a), Mb3Cas12a (Moraxella bovoculi Cas12a), FnCas12a (Francisella novicida Cas12a), FnoCas12a (Francisella tularensis subsp. novicida FTG Cas12a), FbCas12a (Flavobacteriales bacterium Cas12a), Lb4Cas12a (Lachnospira eligens Cas12a), MbCas12a (Moraxella bovoculi Cas12a), Pb2Cas12a (Prevotella bryantii Cas12a), PgCas12a (Candidatus Parcubacteria bacterium Cas12a), AaCas12a (Acidaminococcus sp. Cas12a), BoCas12a (Bacteroidetes bacterium Cas12a), CMaCas12a (Candidatus Methanomethylophilus alvus CMx1201 Cas12a), and to-be-discovered equivalent Cas12a nucleic acid-guided nucleases and homologs and orthologs of these nucleic acid-guided nucleases (and other nucleic acid-guided nucleases that exhibit both cis-cleavage and trans-cleavage activity.

Guide RNA (gRNA)

The present disclosure detects a target nucleic acid of interest via a reaction mixture containing at least two guide RNAs (gRNAs) (i.e., gRNA1 and gRNA2) each incorporated into a different RNP complex (i.e., RNP1 and RNP2). Suitable gRNAs include at least one crRNA region to enable specificity in every reaction. The gRNA1s of the RNP1s are specific to target nucleic acids of interest and the gRNA2s of the RNP2s are specific to an unblocked nucleic acid, a synthesized activating molecule, or an RNP2 activating nucleic acid depending on the embodiment of the signal boost assay, all of which are described in detail below. As will be clear given the description below, an advantageous feature of the signal boost assay is that, with the exception of the gRNA1s in the RNP1s (i.e., the gRNAs specific to the target nucleic acids of interest), the signal boost assay components can stay the same (i.e., are identical or substantially identical) no matter what target nucleic acids of interest are being detected, and the gRNA1s in the RNP1s are easily reprogrammable using known techniques and gRNA design tools.

Like the nucleic acid-guided nuclease, the gRNA may be provided in the signal boost assay reaction mix in a preassembled RNP, as an RNA molecule, or may also be provided as a DNA sequence to be transcribed, in, e.g., a vector backbone. Providing the gRNA in a pre-assembled RNP complex (i.e., RNP1 or RNP2) is preferred if rapid kinetics are preferred. If provided as a gRNA molecule, the gRNA sequence may include multiple endoribonuclease recognition sites (e.g., Csy4) for multiplex processing. Alternatively, if provided as a DNA sequence to be transcribed, an endoribonuclease recognition site may be encoded between neighboring gRNA sequences such that more than one gRNA can be transcribed in a single expression cassette. Direct repeats can also serve as endoribonuclease recognition sites for multiplex processing. Guide RNAs are generally about 20 nucleotides to about 300 nucleotides in length and may contain a spacer sequence containing a plurality of bases and complementary to a protospacer sequence in the target sequence. The gRNA spacer sequence may be 50%, 60%, 75%, 80%, 85%, 90%, 95%, 97.5%, 98%, 99%, or more complementary to its intended target nucleic acid of interest.

The gRNA of RNP1 is capable of binding to the nucleic acid-guided nuclease of RNP1 to perform cis-cleavage of a target nucleic acid of interest (e.g., a DNA or RNA), where the binding also triggers non-sequence specific trans-cleavage of other molecules in the reaction mix. Guide RNAs include any polynucleotide sequence having sufficient complementarity with a target nucleic acid of interest (or target sequences generated by unblocking blocked nucleic acid molecules or target sequences generated by synthesizing synthesized activating molecules as described below). Target nucleic acids of interest (described above) preferably include a protospacer-adjacent motif (PAM), and, following gRNA binding, the nucleic acid-guided nuclease induces a double-stranded break either inside or outside the protospacer region of the target nucleic acid of interest.

In some embodiments, the gRNA (e.g., of RNP1) is an exo-resistant circular molecule that can include several DNA bases between the 5′ end and the 3′ end of a natural guide RNA and is capable of binding a target sequence. The length of the circularized guide for RNP1 can be such that the circular form of guide can be complexed with a nucleic acid-guided nuclease to form a modified RNP1 which can still retain its cis-cleavage i.e., (specific) and trans-cleavage (i.e., non-specific) nuclease activity.

In any of the foregoing embodiments, the gRNA may be a modified or non-naturally occurring nucleic acid molecule. In some embodiments, the gRNAs of the disclosure may further contain a locked nucleic acid (LNA), a bridged nucleic acid (BNA), and/or a peptide nucleic acid (PNA). By way of further example, a modified nucleic acid molecule may contain a modified or non-naturally occurring nucleoside, nucleotide, and/or internucleoside linkage, such as a 2′-O-methyl(2′-O-Me) modified nucleoside, a 2′-fluoro (2′-F) modified nucleoside, and a phosphorothioate (PS) bond, or any other nucleic acid molecule modifications described herein.

### Ribonucleoprotein (RNP) Complexes

Although the signal boost assay “reaction mix” or “reaction mixture” may comprise separate nucleic acid-guided nucleases and gRNAs (or coding sequences therefor), the signal boost assays preferably comprise preassembled ribonucleoprotein complexes (RNPs) in the reaction mix, allowing for faster detection kinetics. The present signal boost assay employs at least two types of RNP complexes—RNP1 and RNP2—each type containing a nucleic acid-guided nuclease and a gRNA. RNP1 and RNP2 may comprise the same nucleic acid-guided nuclease or may comprise different nucleic acid-guided nucleases; however, the gRNAs in RNP1 and RNP2 are different and are configured to detect different nucleic acids. In some embodiments, the reaction mixture contains about 1 fM to about 10 μM of a given RNP1, or about 1 pM to about 1 μM of a given RNP1, or about 10 pM to about 500 pM of a given RNP1. In some embodiments the reaction mixture contains about 6×10⁴ to about 6×10¹² complexes per microliter (μl) of a given RNP1, or about 6×10⁶ to about 6×10¹⁰ complexes per microliter (μl) of a given RNP1. In some embodiments, the reaction mixture contains about 1 fM to about 500 μM of a given RNP2, or about 1 pM to about 250 μM of a given RNP2, or about 10 pM to about 100 μM of a given RNP2. In some embodiments the reaction mixture contains about 6×10⁴ to about 6×10¹² complexes per microliter (μl) of a given RNP2 or about 6×10⁶ to about 6×10¹² complexes per microliter (μl) of a given RNP2. See Example II below describing preassembling RNPs and Examples V and VI below describing various signal boost assay conditions.

In any of the embodiments of the disclosure, 1 to about 1,000 different RNP1s may be used to interrogate target nucleic acids (e.g., 1, 2, 3, 4, 5, 6, 7, 8, 9, 10, 11, 12, 13, 14, 15, 16, 27, 28, 19, 20, 21, 22, 23, 24, 25, 50, 75, 100, 125, 150, 175, 200, 250, 300, 350, 400, 450, 500, 550, 600, 650, 700, 750, 800, 850, 900, 950, 1,0000, or 5,000 or more RNP1s), where different RNP1s comprise a different gRNA polynucleotide sequence. That is, more than one RNP1 may be present for the purpose of targeting one target nucleic acid of interest from each of many sources or more than one (e.g., several to many) RNP1s may be present for the purpose of targeting more than one target nucleic acids of interest from a single source organism (or source chromosome, source cell, source tissue, etc.).

In any of the foregoing embodiments and as contemplated herein, the gRNA1 of a specific RNP1 may be homologous or heterologous, relative to the gRNA of other RNP1(s) present in the reaction mixture. A homologous mixture of RNP1 gRNA1s has a number of gRNA1s with the same nucleotide sequence, whereas a heterologous mixture of RNP1 gRNA1s has multiple gRNA1s with different nucleotide sequences (e.g., gRNAs targeting different loci, genes, variants, and/or source organisms). There will be many RNP1s with the same gRNA1; however, there will be many RNP1s with different gRNA1 sequences. Therefore, the disclosed methods may include a reaction mixture containing more than two heterologous gRNA1s, more than three heterologous gRNA1s, more than four heterologous gRNA1s, more than five heterologous gRNA1s, more than six heterologous gRNA1s, more than seven heterologous gRNA1s, more than eight heterologous gRNA1s, more than nine heterologous gRNA1s, more than ten heterologous gRNAs, more than eleven heterologous gRNA1s, more than twelve heterologous gRNA1s, more than thirteen heterologous gRNA1s, more than fourteen heterologous gRNA1s, more than fifteen heterologous gRNA1s, more than sixteen heterologous gRNA1s, more than seventeen heterologous gRNA1s, more than eighteen heterologous gRNA1s, more than nineteen heterologous gRNA1s, more than twenty heterologous gRNA1s, more than twenty-one heterologous gRNA1s, more than twenty-three heterologous gRNA1s, more than twenty-four heterologous gRNA1s, more than twenty-five heterologous gRNA1s, more than fifty heterologous gRNA1s, more than one hundred heterologous gRNA1s, more than five hundred heterologous gRNA1s, more than one thousand heterologous gRNA1s, or more than five thousand gRNA1s.

As a first non-limiting example of a heterologous mixture of RNP1 gRNAs, the reaction mixture may contain: a number of RNP1s (RNP1-1s) having a gRNA targeting parainfluenza virus 1; a number of RNP1s (RNP1-2s) having a gRNA targeting human metapneumovirus; a number of RNP1s (RNP1-3s) having a gRNA targeting human rhinovirus; a number of RNP1s (RNP1-4s) having a gRNA targeting human enterovirus; and a number of RNP1s (RNP1-5s) having a gRNA targeting coronavirus HKU1. As a second non-limiting example of a heterologous mixture of RNP1 gRNAs, the reaction mixture may contain: a number of RNP1s containing a gRNA targeting two or more SARS-Co-V-2 variants, e.g., B.1.1.7, B.1.351, P.1, B.1.617.2, BA.1, BA.2, BA.2.12.1, BA.4, and BA.5 and subvariants thereof.

As another non-limiting example of a heterologous mixture of RNP1 gRNAs, the reaction mixture may contain RNP1s targeting two or more target nucleic acids of interest from organisms that infect grapevines, such as Guignardia bidwellii (RNP1-1), Uncinula necator (RNP1-2), Botrytis cincerea (RNP1-3), Plasmopara viticola (RNP1-4), and Botryotinis fuckleina (RNP1-5).

### Reporter Moieties

The signal boost assay detects a target nucleic acid of interest via detection of a signal generated in the reaction mix by a reporter moiety. In many embodiments the detection of the target nucleic acids of interest occurs within ten minutes including sample prep. Reporter moieties can comprise DNA, RNA, a chimera of DNA and RNA, and can be single stranded, double stranded, or a moiety that is a combination of single stranded portions and double stranded portions.

Depending on the type of reporter moiety used, trans- and/or cis-cleavage by the nucleic acid-guided nuclease in RNP2 releases a signal. In some embodiments, trans-cleavage of stand-alone reporter moieties (e.g., not bound to any blocked nucleic acid molecules or blocked primer molecules) may generate signal changes at rates that are proportional to the cleavage rate, as new RNP2s are activated over time (shown in FIGS. 1D, 2A, 3A, 3B and 4 at bottom, and at top of FIG. 5). Trans-cleavage by either an activated RNP1 or an activated RNP2 may release a signal although the vast majority of trans-cleavage of the reporter moieties are due to the trans-cleavage activity of RNP2. In alternative embodiments and preferably, the reporter moiety may be bound to the blocked nucleic acid molecule, where trans-cleavage of the blocked nucleic acid molecule (or blocked primer molecule) and conversion to an unblocked nucleic acid molecule (or unblocked primer molecule) may generate signal changes at rates that are proportional to the cleavage rate as new RNP2s are activated over time, thus allowing for real time reporting of results (shown at FIG. 5, center). In yet another embodiment, the reporter moiety may be bound to a blocked nucleic acid molecule such that cis-cleavage following the binding of the RNP2 to an unblocked nucleic acid molecule releases a PAM distal sequence, which in turn generates a signal at rates that are proportional to the cleavage rate (shown at FIG. 5, bottom). In this case, activation of RNP2 by cis-(target specific) cleavage of the unblocked nucleic acid molecule directly produces a signal, rather than producing a signal via indiscriminate trans-cleavage activity. Alternatively or in addition, a reporter moiety may be bound to the gRNA.

The reporter moiety may be a synthetic molecule linked or conjugated to a reporter and quencher such as, for example, a TaqMan probe with a dye label (e.g., FAM or FITC) on the 5′ end and a quencher on the 3′ end. The reporter and quencher may be about 20-30 bases apart or less (i.e., 10-11 nm apart or less) for effective quenching via fluorescence resonance energy transfer (FRET). Alternatively, signal generation may occur through different mechanisms. Other detectable moieties, labels, or reporters can also be used to detect a target nucleic acid of interest as described herein. Reporter moieties can be labeled in a variety of ways, including direct or indirect attachment of a detectable moiety such as a fluorescent moiety, hapten, or colorimetric moiety.

Examples of detectable moieties include various radioactive moieties, enzymes, prosthetic groups, fluorescent markers, luminescent markers, bioluminescent markers, metal particles, and protein-protein binding pairs, e.g., protein-antibody binding pairs. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, and phycoerythrin. Examples of bioluminescent markers include, but are not limited to, luciferase (e.g., bacterial, firefly, click beetle and the like), luciferin, and aequorin. Examples of enzyme systems having visually detectable signals include, but are not limited to, galactosidases, glucorinidases, phosphatases, peroxidases, and cholinesterases. Identifiable markers also include radioactive elements such as ¹²⁵I, ³⁵S, ¹⁴C, or ³H. Reporters can also include a change in pH or charge of the signal boost assay reaction mix.

The methods used to detect the generated signal will depend on the reporter moiety or moieties used. For example, a radioactive label can be detected using a scintillation counter, photographic film as in autoradiography, or storage phosphor imaging. Fluorescent labels can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Enzymatic labels can be detected by providing the appropriate substrates for the enzyme and detecting the resulting reaction product. Simple colorimetric labels can be detected by observing the color associated with the label. When pairs of fluorophores are used in an assay, fluorophores are chosen that have distinct emission patterns (wavelengths) so that they can be easily distinguished.

Single-stranded, double-stranded or reporter moieties comprising both single- and double-stranded portions can be introduced to show a signal change proportional to the cleavage rate, which increases with every new activated RNP2 complex over time. In some embodiments and as described in detail below, reporter moieties can also be embedded into the blocked nucleic acid molecules (or blocked primer molecules) for real time reporting of results.

For example, the method of detecting a target nucleic acid of interest in a sample using a signal boost assay as described herein can involve contacting the reaction mix with a labeled detection ssDNA containing a fluorescent resonance energy transfer (FRET) pair, a quencher/phosphor pair, or both. A FRET pair consists of a donor chromophore and an acceptor chromophore, where the acceptor chromophore may be a quencher molecule. FRET pairs (donor/acceptor) suitable for use include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5, Texas Red/DABCYL, BODIPY/DABCYL, Lucifer yellow/DABCYL, coumarin/DABCYL, and fluorescein/LC Red 705. In addition, a fluorophore/quantum dot donor/acceptor pair can be used. EDANS is (5-((2-Aminoethyl)amino) naphthalene-1-sulfonic acid); IAEDANS is 5-({2-[(iodoacetyl)amino]ethyl}amino) naphthalene-1-sulfonic acid); DABCYL is 4-(4-dimethylaminophenyl)diazenylbenzoic acid. Useful quenchers include, but are not limited to, BHQ, DABCYL, QSY 7 and QSY 33.

In any of the foregoing embodiments, the reporter moiety may comprise one or more modified nucleic acid molecules, containing a modified nucleoside or nucleotide. In some embodiments the modified nucleoside or nucleotide is chosen from 2′-O-methyl(2′-O-Me) modified nucleoside, a 2′-fluoro (2′-F) modified nucleoside, and a phosphorothioate (PS) bond, or any other nucleic acid molecule modifications described below.

### Nucleic Acid Modifications

For any of the nucleic acid molecules described herein (e.g., blocked nucleic acid molecules, blocked primer molecules, gRNAs, template molecules, synthesized activating molecules, RNP2 activating nucleic acids and reporter moieties), the nucleic acid molecules may be used in a wholly or partially modified form. Typically, modifications to the blocked nucleic acid molecules, gRNAs, template molecules, reporter moieties, and blocked primer molecules described herein are introduced to optimize the molecule's biophysical properties (e.g., increasing nucleic acid-guided nuclease resistance and/or increasing thermal stability). Modifications typically are achieved by the incorporation of, for example, one or more alternative nucleosides, alternative sugar moieties, and/or alternative internucleoside linkages.

For example, one or more of the signal boost assay components may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The nucleic acid molecules described herein (e.g., blocked nucleic acid molecules, blocked primer molecules, gRNAs, reporter molecules, synthesized activating molecules, and template molecules) may also include nucleobases in which the purine or pyrimidine base is replaced with other heterocycles, for example 7-deaza-adenine, 7-deazaguanosine, 2-aminopyridine, and/or 2-pyridone. Further modification of the nucleic acid molecules described herein may include nucleobases disclosed in U.S. Pat. No. 3,687,808; Kroschwitz, ed., The Concise Encyclopedia of Polymer Science and Engineering, NY, John Wiley & Sons, 1990, pp. 858-859; Englisch, et al., Angewandte Chemie, 30:613 (1991); and Sanghvi, Chapter 16, Antisense Research and Applications, CRC Press, Gait, ed., 1993, pp. 289-302.

In addition to or as an alternative to nucleoside modifications, the signal boost assay components may comprise 2′ sugar modifications, including 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can modify the nucleic acid molecules described herein (i.e., blocked nucleic acid molecules, gRNAs, synthesized activating molecules, reporter molecules, and blocked primer molecules) may include all possible orientations of OH; F; O—, S—, or N-alkyl (mono- or di-); O—, S—, or N-alkenyl (mono- or di-); O-, S- or N-alkynyl (mono- or di-); or O-alkyl-O-alkyl, wherein the alkyl, alkenyl and alkynyl may be substituted or unsubstituted C₁ to C₁₀ alkyl or C₂ to C₁₀ alkenyl and alkynyl. Other potential sugar substituent groups include, e.g., aminopropoxy (—OCH₂CH₂CH₂NH₂), allyl (—CH₂—CH—CH₂),—O-allyl (—O—CH₂—CH—CH₂) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

Finally, modifications to the signal boost assay components may comprise internucleoside modifications such as phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### The Signal Boost Assay Employing Blocked Nucleic Acid Molecules

As described above in relation to FIGS. 1A and 1B, the signal boost assay is performed in partitions in assay modules, where each partition comprises specific, known RNP1s. FIG. 1D, described above, depicts the signal boost assay generally. A specific embodiment of the signal boost assay utilizing blocked nucleic acid molecules is depicted in FIG. 2A and described in detail below. In this embodiment, a blocked nucleic acid is used to prevent the activation of RNP2 in the absence of a target nucleic acid of interest. The method in FIG. 2A begins with providing the signal boost assay components RNP1 (201), RNP2 (202) and blocked nucleic acid molecules (203). RNP1 (201) comprises a gRNA specific for a target nucleic acid of interest and a nucleic acid-guided nuclease (e.g., Cas 12a or Cas 14 for a DNA target nucleic acid of interest or a Cas 13a for an RNA target nucleic acid of interest) and RNP2 (202) comprises a gRNA specific for an unblocked nucleic acid molecule and a nucleic acid-guided nuclease (again, e.g., Cas 12a or Cas 14 for a DNA unblocked nucleic acid molecule or a Cas 13a for an RNA unblocked nucleic acid molecule). As described above, the nucleic acid-guided nucleases in RNP1 (201) and RNP2 (202) can be the same or different depending on the type of target nucleic acid of interest and unblocked nucleic acid molecule. What is key, however, is that the nucleic acid-guided nucleases in RNP1 and RNP2 are activated to have trans-cleavage activity following target nucleic acid or unblocked nucleic acid molecule binding and/or initiation of cis-cleavage activity.

In a first step, a sample—in the present case, an aliquot of a sample—comprising a target nucleic acid of interest (204) is added to the signal boost assay reaction mix. Keep in mind that although shown as a single reaction, the method depicted in FIG. 2A is performed in several to many to a large number of partitions in parallel, where each partition comprises different RNP1s. The target nucleic acid of interest (204) combines with and activates RNP1 (205) but does not interact with or activate RNP2 (202). Once activated, RNP1 binds the target nucleic acid of interest (204) and cuts the target nucleic acid of interest (204) via sequence-specific cis-cleavage, activating non-specific trans-cleavage of other nucleic acids present in the reaction mix, including the blocked nucleic acid molecules (203). At least one of the blocked nucleic acid molecules (203) becomes an unblocked nucleic acid molecule (206) when the blocking moiety (207) is removed. As described below, “blocking moiety” may refer to nucleoside modifications, topographical configurations such as secondary structures, and/or structural modifications.

Once at least one of the blocked nucleic acid molecules (203) is unblocked, the unblocked nucleic acid molecule (206) can then bind to and activate an RNP2 (208). Because the nucleic acid-guided nucleases in the RNP1s (205) and RNP2s (208) have both cis- and trans-cleavage activity, cis-cleavage activity cuts the unblocked nucleic acid molecule and the trans-cleavage activity causes more blocked nucleic acid molecules (203) become unblocked nucleic acid molecules (206) triggering activation of even more RNP2s (208) and more trans-cleavage activity in a cascade. FIG. 2A at bottom depicts the concurrent activation of reporter moieties. Intact reporter moieties (209) comprise a quencher (210) and a fluorophore (211) linked by a nucleic acid sequence. As described above in relation to FIG. 1D, the reporter moieties are also subject to trans-cleavage by activated RNP1 (205) and RNP2 (208). The intact reporter moieties (209) become activated reporter moieties (212) when the quencher (210) is separated from the fluorophore (211), emitting a fluorescent signal (213). Signal strength increases rapidly as more blocked nucleic acid molecules (203) become unblocked nucleic acid molecules (206) triggering cis-cleavage activity of more RNP2s (208) and thus more trans-cleavage activity of the reporter moieties (209). Again, the reporter moieties are shown here as separate molecules from the blocked nucleic acid molecules, but other configurations may be employed and are discussed in relation to FIG. 5. One particularly advantageous feature of the signal boost assay is that, with the exception of the gRNA in the RNP1 (gRNA1), the signal boost assay components are modular in the sense that the components can stay the same no matter what target nucleic acids of interest are being detected.

FIG. 2B is a diagram showing an exemplary blocked nucleic acid molecule (220) and an exemplary technique for unblocking the blocked nucleic acid molecules described herein. A blocked single-stranded or double-stranded, circular or linear, DNA or RNA molecule (or a combination of DNA and RNA) (220) comprising a target strand (222) may contain a partial hybridization with a complementary non-target strand nucleic acid molecule (224) containing unhybridized and cleavable secondary loop structures (226) (e.g., hairpin loops, tetraloops, pseudoknots, junctions, kissing hairpins, internal loops, bulges, and multibranch loops). Trans-cleavage of the loops by, e.g., activated RNP1s or RNP2s, generates short strand nucleotide sequences or regions (228) which, because of the short length and low melting temperature Tₘ can dehybridize at room temperature (e.g., 15°−25° C.), thereby unblocking the blocked nucleic acid molecule (220) to create an unblocked nucleic acid molecule (230), enabling the internalization of the unblocked nucleic acid molecule (230) (target strand) into an RNP2, leading to RNP2 activation.

A blocked nucleic acid molecule may be single-stranded or double-stranded, circular or linear, and may further contain a partially hybridized nucleic acid sequence containing cleavable secondary loop structures, as exemplified in FIG. 2B. Such blocked nucleic acid molecules typically have a low binding affinity, or high dissociation constant (K_{d}) in relation to binding to RNP2 and may be referred to herein as a high K_{d} nucleic acid molecule. In the context of the present disclosure, the binding of blocked or unblocked nucleic acid molecules or blocked or unblocked primer molecules to RNP2, low K_{d} values range from about 100 fM to about 1 aM or lower (e.g., 100 zM) and high K_{d} values are in the range of 100 nM to about 10-100 10 mM and thus are about 10⁵-, 10⁶-, 10⁷-, 10⁸-, 10⁹- to 10¹⁰-fold or higher as compared to low K_{d} values. Of course, the ideal blocked nucleic acid molecule would have an “infinite K_{d}.”

The blocked nucleic acid molecules (high K_{d} molecules) described herein can be converted into unblocked nucleic acid molecules (low K_{d} molecules—also in relation to binding to RNP2) via cleavage of nuclease-cleavable regions (e.g., via active RNP1s and RNP2s). The unblocked nucleic acid molecule has a higher binding affinity for the gRNA in RNP2 than does the blocked nucleic acid molecule.

Once the unblocked nucleic acid molecule is bound to RNP2, the RNP2 activation triggers trans-cleavage activity, which in turn leads to more RNP2 activation by further cleaving blocked nucleic acid molecules, resulting in a positive feedback loop or cascade.

In embodiments where blocked nucleic acid molecules are linear and/or form a secondary structure, the blocked nucleic acid molecules may be single-stranded (ss) or double-stranded (ds) and contain a first nucleotide sequence and a second nucleotide sequence. The first nucleotide sequence has sufficient complementarity to hybridize to a gRNA of RNP2, and the second nucleotide sequence does not. The first and second nucleotide sequences of a blocked nucleic acid molecule may be on the same nucleic acid molecule (e.g., for single-strand embodiments) or on separate nucleic acid molecules (e.g., for double-strand embodiments). Trans-cleavage (e.g., via RNP1 or RNP2) of the second nucleotide sequence converts the blocked nucleic acid molecule to a single-strand unblocked nucleic acid molecule. The unblocked nucleic acid molecule contains only the first nucleotide sequence, which has sufficient complementarity to hybridize to the gRNA of RNP2, thereby activating the trans-cleavage activity of RNP2.

The second nucleotide sequence at least partially hybridizes to the first nucleotide sequence, resulting in a secondary structure containing at least one loop (e.g., hairpin loops, tetraloops, pseudoknots, junctions, kissing hairpins, internal loops, bulges, and multibranch loops). Such loops block the nucleic acid molecule from binding or incorporating into an RNP complex thereby initiating cis- or trans-cleavage.

In some embodiments, the blocked nucleic acid molecule may contain a protospacer adjacent motif (PAM) sequence, or partial PAM sequence, positioned between the first and second nucleotide sequences, where the first sequence is 5′ to the PAM sequence, or partial PAM sequence. Inclusion of a PAM sequence may increase the reaction kinetics internalizing the unblocked nucleic acid molecule into RNP2 and thus decrease the time to detection. In other embodiments, the blocked nucleic acid molecule does not contain a PAM sequence. In some embodiments, the blocked nucleic acid molecules are configured as described in U.S. Pat. Nos. 11,639,520; 11,702,686; 11,821,025; 11,970,730; 11,884,921; 11,820,983.

Nucleotide mismatches can be introduced in double-strand regions of the blocked nucleic acid molecules to reduce the melting temperature (Tm) of the region (i.e., in the “clamp” regions) such that once the loop is cleaved, the double-strand segment is unstable and dehybridizes rapidly. The percentage of nucleotide mismatches of a given region may vary between 0% and 50%; however, the maximum number of nucleotide mismatches is limited to a number where the secondary loop structure still forms. In other words, the number of hybridized bases can be less than or equal to the length of each double-strand segment and vary based on number of mismatches introduced.

In any of the foregoing embodiments, the blocked nucleic acid molecules of the disclosure may further contain a reporter moiety attached thereto such that cleavage of the blocked nucleic acid releases a signal from the reporter moiety. (See FIG. 5, mechanisms depicted at center and bottom.)

Also, in any of the foregoing embodiments, the blocked nucleic acid molecule may be a modified or non-naturally occurring nucleic acid molecule. In some embodiments, the blocked nucleic acid molecules of the disclosure may further contain a locked nucleic acid (LNA), a bridged nucleic acid (BNA), and/or a peptide nucleic acid (PNA). The blocked nucleic acid molecule may contain a modified or non-naturally occurring nucleoside, nucleotide, and/or internucleoside linkage, such as a 2′-O-methyl(2′-O-Me) modified nucleoside, a 2′-fluoro (2′-F) modified nucleoside, and a phosphorothioate (PS) bond, any other nucleic acid molecule modifications described above, and any combination thereof.

In some embodiments, the blocked nucleic acid molecules are circular DNAs, RNAs or chimeric (DNA-RNA) molecules, and the blocked nucleic acid molecules may include different base compositions depending on the Cas enzyme used for RNP1 and RNP2. For the circular design of blocked nucleic acid molecules, the 5′ and 3′ ends are covalently linked together. This configuration makes internalization of the blocked nucleic acid molecule into RNP2—and subsequent RNP2 activation—sterically unfavorable, thereby blocking the progression of the signal boost assay. Thus, RNP2 activation (e.g., trans-cleavage activity) happens after cleavage of a portion of the blocked nucleic acid molecule followed by linearization and internalization of unblocked nucleic acid molecule into RNP2.

In some embodiments, the blocked nucleic acid molecules are topologically circular molecules with 5′ and 3′ portions hybridized to each other using DNA, RNA, LNA, BNA, or PNA bases which have a very high melting temperature (Tm). The high Tm causes the structure to effectively behave as a circular molecule even though the 5′ and 3′ ends are not covalently linked. The 5′ and 3′ ends can also have base non-naturally occurring modifications such as phosphorothioate bonds to provide increased stability.

In embodiments where the blocked nucleic acid molecules are circularized (e.g., circular or topologically circular), each blocked nucleic acid molecule includes a first region, which is a target sequence specific to the gRNA of RNP2, and a second region, which is a sequence that can be cleaved by nuclease enzymes of activated RNP1 and/or RNP2. The first region may include a nuclease-resistant nucleic acid sequence such as, for example, a phosphorothioate group or other non-naturally occurring nuclease-resistant base modifications, for protection from trans-nucleic acid-guided nuclease activity. In some embodiments, when the Cas enzyme in both RNP1 and RNP2 is Cas12a, the first region of the blocked nucleic acid molecule includes a nuclease-resistant DNA sequence, and the second region of the blocked nucleic acid molecule includes a cleavable DNA sequence. In other embodiments, when the Cas enzyme in RNP1 is Cas12a and the Cas enzyme in RNP2 is Cas13a, the first region of the blocked nucleic acid molecule includes a nuclease-resistant RNA sequence, and the second region of the blocked nucleic acid molecule includes a cleavable DNA sequence and a cleavable RNA sequence. In yet other embodiments, when the Cas enzyme in RNP1 is Cas13a and the Cas enzyme in RNP2 is Cas12a, the first region of the blocked nucleic acid molecule includes a nuclease-resistant DNA sequence, and the second region of the blocked nucleic acid molecule includes a cleavable DNA sequence and a cleavable RNA sequence. In some other embodiments, when the Cas enzyme in both RNP1 and RNP2 is Cas13a, the first region of the blocked nucleic acid molecule includes a nuclease-resistant RNA sequence, and the second region of the blocked nucleic acid molecule includes a cleavable RNA sequence.

### The Signal Boost Assay Employing Blocked Primer Molecules

The blocked nucleic acid molecules described above may also be blocked primer molecules. Blocked primer molecules may be configured identically to or similarly to blocked nucleic acid molecules as described above, except blocked primer molecules include a sequence complementary to a primer binding domain (PBD) on a template molecule (see description below in reference to FIGS. 3A and 3B). An unblocked primer nucleic acid molecule can bind to a template molecule at the PBD and copy the template molecule via polymerization by a polymerase.

Specific embodiments of the signal boost assay utilize blocked primer molecules and are depicted in FIGS. 3A and 3B. As with the embodiment of the signal boost assay shown in FIG. 2A, keep in mind that in the massively multiplexed signal boost assays described herein, the single reaction method depicted in FIGS. 3A and 3B is performed in many reactions in separate partitions in parallel, wherein each partition comprises different RNP1s. In the embodiments using blocked nucleic acid molecules described above, activation of RNP1 by binding of N nucleotides of the target nucleic acid molecules or cis-cleavage of the target nucleic acid molecules initiates trans-cleavage of the blocked nucleic acid molecules which were used to activate RNP2—that is, the unblocked nucleic acid molecules are a target sequence for the gRNA in RNP2. In contrast, in the embodiments using blocked primers, activation of RNP1 and trans-cleavage unblocks a blocked primer molecule that is then used to prime a template molecule for extension by a polymerase, thereby synthesizing synthesized activating molecules that are the target sequence for the gRNA in RNP2.

FIG. 3A is a diagram showing the sequence of steps in an exemplary signal boost assay involving circular blocked primer molecules and linear template molecules. At left of FIG. 3A is a signal boost assay reaction mix comprising 1) RNP1s (301) (only one RNP1 is shown); 2) RNP2s (302); 3) linear template molecules (330) (which is the non-target strand); 4) a circular blocked primer molecule (334) (i.e., a high K_{d} molecule); and 5) a polymerase (338), such as a Φ29 polymerase. The linear template molecule (330) (non-target strand) comprises a PAM sequence (331), a primer binding domain (PBD) (332) and, optionally, a nucleoside modification (333) to protect the linear template molecule (330) from 3′→5′ exonuclease activity. Blocked primer molecule (334) comprises a cleavable region (335) and a complement to the PBD (332) on the linear template molecule (330).

Upon addition of a sample comprising a target nucleic acid of interest (304) (capable of complexing with the gRNA in RNP1 (301)), the target nucleic acid of interest (304) is bound with and activates RNP1 (305) but does not interact with or activate RNP2 (302). Once activated, RNP1 cuts the target nucleic acid of interest (304) via sequence specific cis-cleavage, which activates non-specific trans-cleavage of other nucleic acids present in the reaction mix, including at least one of the blocked primer molecules (334). The circular blocked primer molecule (334) (i.e., a high K_{d} molecule, where high K_{d} relates to binding to the template molecules) upon cleavage becomes an unblocked linear primer molecule (344) (a low K_{d} molecule, where low K_{d} relates to binding to the template molecules), which has a region (336) complementary to the PBD (332) on the linear template molecule (330) and thus the unblocked linear primer molecule (344) can bind to the linear template molecule (330).

Once the unblocked linear primer molecule (344) and the linear template molecule (330) are hybridized (i.e., hybridized at the PBD (332) of the linear template molecule (330) and the PBD complement (336) on the unblocked linear primer molecule (344)), 3′→5′ exonuclease activity of the polymerase (338) removes the unhybridized single-stranded DNA at the end of the unblocked primer molecule (344) and the polymerase (338) can copy the linear template molecule (330) to produce a synthesized activating molecule (346) which is a complement of the template molecule (i.e., the non-target strand), thus the synthesized activating molecule (346) is the target strand.

The synthesized activating molecule (346) is capable of activating RNP2 (302→308). As described above, because the nucleic acid-guided nuclease in the RNP2 (308) complex exhibits (that is, possesses) both cis- and trans-cleavage activity, more blocked primer molecules (334) become unblocked primer molecules (344) triggering activation of more RNP2s (308) and more trans-cleavage activity in a cascade. As stated above in relation to blocked and unblocked nucleic acid molecules (both linear and circular), the unblocked primer molecule has a higher binding affinity for the template molecule (330) than does the blocked primer molecule. However, an unblocked primer molecule has a substantially higher likelihood than a blocked primer molecule to hybridize with the template molecule (330).

FIG. 3A at bottom depicts the concurrent activation of reporter moieties. Intact reporter moieties (309) comprise a quencher (310) and a fluorophore (311). As described above in relation to FIGS. 1D and 2A, the reporter moieties are also subject to trans-cleavage by activated RNP1 (305) and RNP2 (308). The intact reporter moieties (309) become activated reporter moieties (312) when the quencher (310) is separated from the fluorophore (311), and the fluorophore emits a fluorescent signal (313). Signal strength increases rapidly as more blocked primer molecules (334) become unblocked primer molecules (344) generating synthesized activating molecules (346) and triggering activation of more RNP2 (308) complexes and more trans-cleavage activity of the reporter moieties (309). Again, here the reporter moieties are shown as separate molecules from the blocked nucleic acid molecules, but other configurations may be employed and are discussed in relation to FIG. 5. Also, as with the signal boost assay embodiment utilizing blocked nucleic acid molecules that are not blocked primers, with the exception of the gRNA in RNP1, the signal boost assay components stay the same no matter what target nucleic acid(s) of interest are being detected.

FIG. 3B is a diagram showing the sequence of steps in an exemplary signal boost assay involving circular blocked primer molecules and circular template molecules. The signal boost assay of FIG. 3B differs from that depicted in FIG. 3A by the configuration of the template molecule. Where the template molecule in FIG. 3A was linear, in FIG. 3B the template molecule is circular. At left of FIG. 3B is a signal boost assay reaction mix comprising 1) RNP1s (301) (only one RNP1 is shown); 2) RNP2s (302); 3) a circular template molecule (352) (non-target strand); 4) a circular blocked primer molecule (334); and 5) a polymerase (338), such as a Φ29 polymerase. The circular template molecule (352) (non-target strand) comprises a PAM sequence (331) and a primer binding domain (PBD) (332). Blocked primer molecule (334) comprises a cleavable region (335) and a complement to the PBD (332) on the circular template molecule (352).

Upon addition of a sample comprising a target nucleic acid of interest (304) (capable of complexing with the gRNA in RNP1 (301)), the target nucleic acid of interest (304) binds to and activates RNP1 (305) but does not interact with or activate RNP2 (302). Once activated, RNP1 cuts the target nucleic acid of interest (304) via sequence specific cis-cleavage, which activates non-specific trans-cleavage of other nucleic acids present in the reaction mix, including at least one of the blocked primer molecules (334). The circular blocked primer molecule (334), upon cleavage, becomes an unblocked linear primer molecule (344), which has a region (336) complementary to the PBD (332) on the circular template molecule (352) and can hybridize with the circular template molecule (352).

Once the unblocked linear primer molecule (344) and the circular template molecule (352) are hybridized (i.e., hybridized at the PBD (332) of the circular template molecule (352) and the PBD complement (336) on the unblocked linear primer molecule (344)), 3′→5′ exonuclease activity of the polymerase (338) removes the unhybridized single-stranded DNA at the 3′ end of the unblocked primer molecule (344). The polymerase (338) can now use the circular template molecule (352) (non-target strand) to produce concatenated activating nucleic acid molecules (360) (which are concatenated target strands), which will be cleaved by the trans-cleavage activity of activated RNP1. The cleaved regions of the concatenated synthesized activating molecules (360) (target strand) are capable of activating the RNP2 (302→308) complex.

As described above, because the nucleic acid-guided nuclease in RNP2 (308) comprises both cis- and trans-cleavage activity, more blocked primer molecules (334) become unblocked primer molecules (344) triggering activation of more RNP2s (308) and more trans-cleavage activity in a cascade. FIG. 3B at bottom depicts the concurrent activation of reporter moieties. Intact reporter moieties (309) comprise a quencher (310) and a fluorophore (311). As described above in relation to FIG. 1D, the reporter moieties are also subject to trans-cleavage by activated RNP1 (305) and RNP2 (308). The intact reporter moieties (309) become activated reporter moieties (312) when the quencher (310) is separated from the fluorophore (311), and the fluorescent signal (313) is unquenched and can be detected. Signal strength increases rapidly as more blocked primer molecules (334) become unblocked primer molecules (344) generating synthesized activating nucleic acid molecules and triggering activation of more RNP2s (308) and more trans-cleavage activity of the reporter moieties (309). Again, here the reporter moieties are shown as separate molecules from the blocked nucleic acid molecules, but other configurations may be employed and are discussed in relation to FIG. 5. Also note that as with the other embodiments of the signal boost assay, in this embodiment, with the exception of the gRNA in RNP1, the signal boost assay components optionally may stay the same no matter what target nucleic acid(s) of interest are being detected.

The polymerases used in the “blocked primer molecule” embodiments serve to polymerize a reverse complement strand of the template molecule (non-target strand) to generate a synthesized activating molecule (target strand) as described above. In some embodiments, the polymerase is a DNA polymerase, such as a BST, T4, or Therminator polymerase (New England BioLabs Inc., Ipswich MA., USA). In some embodiments, the polymerase is a Klenow fragment of a DNA polymerase. In some embodiments the polymerase is a DNA polymerase with 5′→3′ DNA polymerase activity and 3′→5′ exonuclease activity, such as a Type I, Type II, or Type III DNA polymerase. In some embodiments, the DNA polymerase, including the Phi29, T7, Q5®, Q5UR, Phusion®, OneTaq®, LongAmp®, Vent®, or Deep Vent® DNA polymerases (New England BioLabs Inc., Ipswich MA., USA), or any active portion or variant thereof. Also, a 3′ to 5′ exonuclease can be separately used if the polymerase lacks this activity.

The Signal Boosting Signal Boost Assay Employing Blocked Guide (gRNA) Molecules

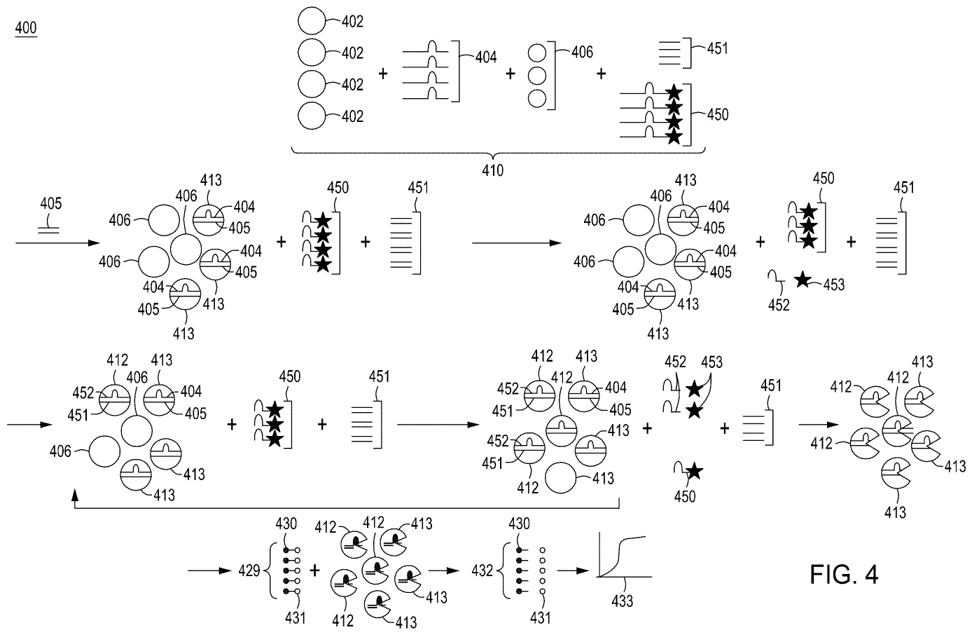

FIG. 4 is a diagram showing the sequence of steps in an exemplary signal boost assay utilizing blocked guide nucleic acid (gRNA) molecules. In this embodiment, instead of a blocked nucleic acid molecule or a blocked primer molecule, a blocked guide molecule (i.e., a blocked guide RNA or blocked gRNA) is used to block activation of RNP2. The blocked guide nucleic acid molecules (blocked gRNA2s) cannot bind to and complex with the second nucleic acid nuclease to form the second ribonucleoprotein complex (RNP2) unless and until the blocked gRNA2s are unblocked via trans-cleavage activity of RNP1. The blocked gRNA2 is complementary to an RNP2 activating nucleic acid. That is, the blocked guide molecule functions like the blocked nucleic acid molecules and the blocked primer molecules to “lock” RNP2 unless and until a target nucleic acid molecule activates RNP1, the trans-cleavage activity of which then unblocks the blocked guide molecules which can then complex with the second nucleic acid-guided nuclease to form second ribonucleoprotein complexes (i.e., RNP2s) which can then be activated. As with the embodiments of the signal boost assay depicted in FIGS. 2A, 3A and 3B, the massively multiplexed signal boost assay depicted in FIG. 4 is performed in several to many partitions in parallel, where each partition comprises different RNP1s.

FIG. 4 is a diagram showing the sequence of steps in an exemplary signal boost assay utilizing blocked guide molecules. In this embodiment, a blocked guide molecule is used to prevent the activation of RNP2 in the absence of activation of RNP1 by a target nucleic acid. The signal boost assay (400) in FIG. 4 begins with providing the signal boost assay components in a reaction mix (410) comprising: 1) first nucleic acid-guided nuclease enzymes (402); 2) first guide nucleic acids (gRNA1) (404); 3) second nucleic acid-guided nuclease enzymes (406); 4) RNP2 activating nucleic acids (451); 5) blocked guide molecules (blocked gRNA2s) (450); and 6) reporter moieties (429) (seen only at bottom of FIG. 4). The RNP2s that will be formed as a result of an activated RNP1 comprise unblocked gRNA2s that are specific for the RNP2 activating nucleic acids (451) and the second nucleic acid-guided nuclease (406) (e.g., Cas12a or Cas14 for DNA RNP2 activating nucleic acids or, e.g., a Cas 13a for RNA RNP2 activating nucleic acids). Both of the nucleic acid-guided nucleases that form RNP1 and RNP2 must, when activated, have trans-cleavage activity following initiation of cis-cleavage activity.

In a first step, the first Cas enzyme (402) is in the reaction mix (410) with the first guide nucleic acids (gRNA1) (404); second nucleic acid-guided nuclease (406); RNP2 activating nucleic acids (451); and blocked guide molecules (450). The first nucleic acid-guided nuclease (402) is complexed with gRNA1 (404) to form RNP1 (413), which then complexes with target nucleic acid molecules (405) to activate cis-cleavage of the target nucleic acid molecules (405). Also seen are second nucleic acid-guided nuclease (406), blocked guide molecules (blocked gRNA2s) (450) and RNP2 activating nucleic acids (451).

Once cis-cleavage of the target nucleic acid molecules (405) occurs, indiscriminate trans-cleavage activity of other nucleic acids in the reaction mix is initiated, including at least one of the blocked gRNA2s (450). The blocked gRNA2s (i.e., a high K_{d} molecules, where high K_{d} relates to binding to the second nucleic acid-guided nuclease (406)) upon cleavage become unblocked gRNA2s 452 (a low K_{d} molecule, where low K_{d} relates to binding to the second nucleic acid-guided nuclease (406)). Thus, at least one of the blocked gRNA2s (450) becomes an unblocked gRNA2 (452) when the blocking moiety (453) is removed from the blocked gRNA2 (450). As described above, “blocking moiety” may refer to nucleoside modifications, topographical configurations such as secondary structures, and/or structural modifications.

Once at least one of the blocked gRNA2s (450) is unblocked, the unblocked gRNA2 (451) can then complex with the second nucleic acid-guided nuclease (406) to form RNP2 (412) which then complexes with RNP2 activating nucleic acids (451) and cleaves the RNP2 activating nucleic acids (451) via cis-cleavage, triggering trans-cleavage of more blocked gRNA2s (450) in the reaction mix (410). Because the nucleic acid-guided nucleases in the activated RNP1s (413) and RNP2s (412) have both cis- and trans-cleavage activity, the trans-cleavage activity causes more blocked gRNA2s (450) to become unblocked gRNA2s (451) triggering activation of even more RNP2s (412) and more trans-cleavage activity in a reaction cascade.

FIG. 4 at bottom depicts the concurrent activation of reporter moieties. Intact reporter moieties (429) comprise a quencher (430) and a fluorophore (431) linked by a nucleic acid sequence. As described above in relation to FIG. 1D, the intact reporter moieties (429) are also subject to trans-cleavage by activated RNP1 (413) and, primarily, RNP2 (412). The intact reporter moieties (429) become unquenched reporter moieties (432) when the quencher (430) is separated from the fluorophore (431), emitting a fluorescent signal (433). Signal strength increases rapidly as more blocked gRNA2s (450) become unblocked gRNA2s (411) triggering cis-cleavage activity of more RNP2s (412) and thus more trans-cleavage activity of the reporter moieties (429). Again, the reporter moieties are shown here as separate molecules from the blocked nucleic acid molecules, but other configurations may be employed and are discussed in relation to FIG. 5. Also note that as with the other embodiments of the signal boost assay, in this embodiment, with the exception of the gRNA in RNP1, the signal boost assay components optionally may stay the same no matter what target nucleic acid(s) of interest are being detected.

### Reporter Moiety Configurations

FIG. 5 illustrates three exemplary embodiments of reporter moieties. FIG. 5 at top shows the mechanism discussed in relation to FIGS. 2A, 3A and 3B. In this embodiment, a reporter moiety (509) is a separate molecule from the blocked nucleic acid molecules present in the reaction mixture. Reporter moiety (509) comprises a quencher (510) and a fluorophore (511). An activated reporter moiety (512) emits a signal from the fluorophore (511) once it has been physically separated from the quencher (510). Again, if the reporter moiety is a separate molecule that is not activated as part of the blocked nucleic acid molecule (or blocked primer molecule), then activation kinetics of the reporter will be more rapid; however, if activation of the reporter moiety is coupled to unblocking of the blocked nucleic acid molecules (or blocked primer molecules), activation kinetics will be slower.

FIG. 5 at center shows a blocked nucleic acid molecule (503), which is also a reporter moiety. In addition to quencher (510) and fluorophore (511), a blocking moiety (507) can be seen (see also blocked nucleic acid molecules 203 in FIG. 2A). Blocked nucleic acid molecule/reporter moiety (553) comprises a quencher (510) and a fluorophore (511). In this embodiment of the signal boost assay, when the blocked nucleic acid molecule (503) is unblocked due to trans-cleavage initiated by the target nucleic acid of interest binding to RNP1, the unblocked nucleic acid molecule (506) also becomes an activated reporter moiety with fluorophore (511) separated from quencher (510). Note both the blocking moiety (507) and the quencher (510) are removed. In this embodiment, reporter signal is directly generated as the blocked nucleic acid molecules become unblocked. Embodiments of this schema can be used to supply the bulky modifications to the blocked nucleic acid molecules described below.

FIG. 5 at the bottom shows that cis-cleavage of an unblocked nucleic acid molecule or a synthesized activating molecule at a PAM distal sequence by RNP2 generates a signal. Shown are activated RNP2 (508), unblocked nucleic acid molecule (561), quencher (510), and fluorophore (511) forming an activated RNP2 with the unblocked nucleic acid/reporter moiety intact (560). Cis-cleavage of the unblocked nucleic acid/reporter moiety (561) results in an activated RNP2 with the reporter moiety activated (562), comprising the activated RNP2 (508), the unblocked nucleic acid molecule with the reporter moiety activated (563), quencher (510) and fluorophore (511). Embodiments of this schema also can be used to supply the bulky modifications to the blocked nucleic acid molecules described below, and in fact a combination of the configurations of reporter moieties shown in FIG. 5 at center and at bottom may be used.

### Assay Modules

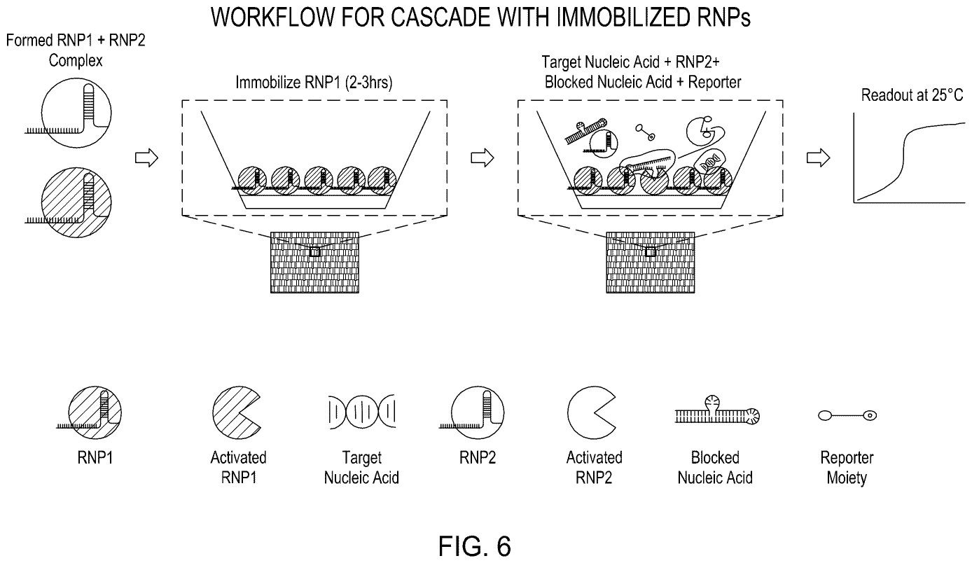

FIG. 6 is a graphic illustration of one embodiment of an exemplary workflow for the 2D or parallel and 3D or array modules where the reactions take place in a partition. As described above, a “partition” may be a well, an isolated region surrounded by interstitial regions or, as described below, a region upon which assay components—specifically RNP1s—are distributed, e.g., on a membrane, beads or in a packed bed of beads. The workflow in FIG. 6 is essentially identical to that shown in and described in relation to FIG. 2A (i.e., the “blocked nucleic acid molecule” embodiment of the signal boost assay).

At left in FIG. 6 are seen preformed ribonucleoprotein complexes RNP1 and RNP2. Moving right, the RNP1s are distributed (or formed) in a partition (here, only one partition is magnified). Following distribution of the RNP1s into the partitions, the sample, RNP2s, blocked nucleic acid molecules and reporter molecules are distributed into the partition and allowed to contact RNP1, where unactivated RNP1s, a target nucleic acid of interest, an activated RNP1, an unactivated RNP2, an activated RNP2, a blocked nucleic acid molecule, and a reporter moiety are all present. When a target nucleic acid of interest activates the trans-cleavage activity of RNP1 by binding to RNP1, unblocking of the blocked nucleic acid molecules is initiated where the unblocked nucleic acid molecules bind to RNP2s activating the trans-cleavage activity of the RNP2s thereby initiating more unblocking of blocked nucleic acid molecules and unquenching reporter moieties in a continuing cascade, thereby “boosting” the signal produced by the reporter moieties.

### 2D or Parallel Module

FIG. 7A is a simplified graphic of an architecture and readout for an exemplary 2D or parallel module. As was seen in FIG. 1C, FIG. 7A is a 2D or parallel module comprising a central well or hub (or “sample splitting zone”) where a sample can be distributed, with ten channels radiating out from the central well leading to intermediate reaction wells and ending in terminal reaction wells. In this 2D or parallel module, a sample is distributed in the center of the assay module, then aliquots of the sample are driven by air displacement using positive or negative pressure into the ten channels and toward the intermediate reaction wells where different RNP1s are distributed at known addresses. Each of the ten intermediate reaction wells comprises a known (and different) RNP1. The RNP1s in some embodiments are lyophilized or air dried for reagent stability and storage. Because the sample is an aqueous-based liquid, as the sample moves into the intermediate reaction wells the lyophilized or air dried RNP1s are reconstituted such that the sample and RNP1 are able to interact if there are target nucleic acids of interest present in the sample.

Once target nucleic acids of interest from the sample, if present, and the RNP1s are able to interact, the RNP1/target nucleic acid complex is driven toward the terminal reaction wells which contain the other assay components—i.e., RNP2, blocked nucleic acids, and reporter moieties—where the signal boost assay takes place. At right in FIG. 7A is an embodiment of the 2D or parallel module where the RNP1 at approximately 4 o'clock is shown to have complexed with a target nucleic acid of interest. As described above, the binding of a single target nucleic acid of interest to RNP1 will initiate unblocking of the blocked nucleic acid molecules via trans-cleavage of the nucleic acid guided nuclease in RNP1 where the unblocked nucleic acid molecules bind to RNP2s initiating trans-cleavage activity of the RNP2s, which in turn cause unblocking of more blocked nucleic acid molecules and unquenching of reporter moieties in a continuing cascade. Imaging will reveal which RNP1s were activated, and thus which target nucleic acid(s) of interest were present in the sample.

In preferred embodiments of the assay modules (both the 2D or parallel and the 3D or array modules), the assay components—i.e., RNP1, RNP2, the blocked nucleic acid molecules, and the reporter moieties—are stored in the assay module in the appropriate well or partition depending on the sequence of steps in which the assay components are added as described in FIGS. 1A and 1B having been lyophilized or air dried. Lyophilization (also known as freeze drying or cryodessication) is a low temperature freeze drying process in which water is removed from a compound after it is frozen and placed under a vacuum, allowing the ice to change directly from solid to vapor without passing through a liquid phase (i.e., by sublimation). Air drying involves allowing a compound (here, the assay components) to be exposed to air so the water evaporates. Typically, air drying for at least 24 hours, or at least 36 hours, or at least 48 hours, or at least 60 hours, or at least 72 hours, or at least 84 hours, depending on the volume of the assay component(s), results in the highest retained activity. See Example VII below. The parameters for lyophilization of the various reagents can be adjusted such that the size of the lyophilized reagent(s) is large enough to be “picked-and-placed” in the partitions of the assay modules and formulated such that reconstitution begins immediately upon contact with the sample.

In an alternative or addition to lyophilization or air drying, some of the assay components (e.g., RNP1 and RNP2) may be immobilized to the surface of the assay module where the surface of the assay module where the assay components are immobilized may comprise features, e.g., such as creases, pillars, fins or other physical properties to increase the surface area for assay component binding. That is, assay components may be disposed on the surface (i.e., in the partitions or wells) of the 2D or parallel module or disposed on functionalized beads or other substrates (generally, “beads”) using, e.g., ligand-receptor interactions or enzyme-mediated reactions, where the beads are then distributed into the partitions. Beads of interest include those fabricated from polyethylene (PE), polyethylene terephthalate (PET), nylon (PA), polypropylene (PA), polystyrene (PS), polymethyl methacrylate (PMMA), glass, silica, and zirconium. In some embodiments, the RNP1s (and one or more other assay components, if applicable) are coupled to the bottom of the partitions/wells or are coupled to beads of size 0.1-100 microns, or 1-100 microns or 1-10 microns in size to optimize retention of the RNP1s via limited diffusion (and one or more other assay components, if applicable) in the proper well. Bead sizes appropriate for the present assay modules are those that are large enough such that diffusion throughout the assay module cannot take place.

The simplest way for assay components to be coupled to a solid support such as the surface of the 2D or parallel module or a bead relies on physical adsorption. That is, RNPs (i.e., RNP1s and/or RNP2s) can be synthesized with an N-terminal or C-terminal peptide of a suitable length with a complementary property to a support to adsorb rapidly to the support; for example, hydrophobic amino acids will adsorb to a hydrophobic material and acidic amino acids will adsorb onto a positively charged surface. This approach is analogous to the immobilization of proteins in ELISA and Western blotting applications that have been used for many years. (See, e.g., Katz, et al., Chem. Soc. Rev., 40:2131-45 (2011).) In another example, nucleic acid-guided nucleases such as Cas 12 and Cas 13 enzymes contain cystine amino acids. Di-sulfide bonds on cystine can be reduced by TCEP, a reducing agent, to create reactive sulfhydryl groups that can then be used for surface binding. Maleimides have covalent affinity for reduced sulfhydryl groups. Therefore, using a maleimide-activated protein like BSA can be used to coat a surface and indirectly immobilize reduced RNPs. In other examples, RNPs can be bound directly to high-bind surfaces.

Another common strategy available to couple the RNPs to a solid support such as the assay module surface or to a bead is to use selective or nonselective chemical reactions to covalently attach peptides to the support. This approach usually requires chemical modification of the surface of the substrate to introduce the relevant functional groups for attachment, with the benefit being the RNPs are covalently attached to the substrate and have no risk of dissociating from the surface during an assay. A variety of reactions have been used to covalently immobilize peptides, including using a nucleophilic α-amino group on the RNP to condense with a carboxylate group on the support; using side chain amino groups on the RNP as a functional handle for coupling to polylysine-coated surfaces; using carboxylate groups on RNPs to react through esterification reactions with the hydroxyl groups presented on cellulose membranes; using amine groups on the RNPs to react with activated succinimidyl ester or isocyanate groups; or selective reaction of thiols with several electrophilic groups. (See, e.g., Geysen, et al., PNAS USA, 81:3998 (1984); Saxinger, et al., BMC Immunol., 6:1.15647109 (2005); and Schutkowski, et al., Agnew. Chem, Int., 43:2671-74 (2004).)

RNPs also can be immobilized using biological strategies, either based on ligand-receptor interactions or enzyme-mediated reactions. The specific noncovalent complex between a biotin tag and avidin or streptavidin is a common example for capturing tagged RNPs onto solid supports. Alternatively, the hybridization of two complementary oligonucleotides can be used to immobilize RNPs, allowing for an array to be “self-assembled.” If complementary oligonucleotides are not employed, presynthesized RNP1s comprising oligonucleotide tags are typically patterned onto a functionalized surface using a robotic liquid handling system. The density of peptide spots prepared using this method depends on the minimum dispensing capacity of the robotic liquid handler, the hydrophobicity of the surface, and solvent evaporation (rapid evaporation leads to incomplete immobilization). The RNP1 arrays can also be fabricated using noncontact inkjet printers or laser printing. (See, e.g., Lesaicherre, et al., Med. Chem. Lett, 12:2079-83 (2002).) In addition, the RNP1 arrays can be fabricated using Cas-antibody coatings.

FIG. 7B is an illustration of an exemplary 2D or parallel module (700). 2D or parallel module (700) comprises an inlet (701), which facilitates fluid displacement via positive or negative pressure; a sample splitting zone (702); twenty fluid channels (703) which connect the sample splitting zone with twenty first reagent wells (704) (i.e., the reagent wells proximal the sample splitting zone); and then connect the first reagent wells (704) with the twenty second reagent wells (705). Also seen are outlets (706), which facilitate fluid displacement (again, via positive or negative pressure), but may also be part of terminal wells used for, e.g., detection. As noted above, the wells in which the reacted assay reagents are detected may be a well in which the final reagent components reside or may be a separate well into which the reacted assay reagents are moved for imaging/detection. Further, here first (704) and second (705) reagent wells are shown to be the same size; however, reagent wells or partitions may be different in size to facilitate mixing, imaging, etc.; for example, if separate wells are used for imaging, the wells may be decreased in size to focus the signal to be detected. Also shown are coupling devices (707), which allow the 2D or parallel modules to be coupled to the pump or plunger or other means for supplying the negative or positive pressure to drive the sample through the fluid channels (703) and/or for coupling the assay module to a sample prep instrumentation and/or a detection and imaging instrumentation.

The sample, when supplied to the sample splitting zone, may be distributed to the fluid channels (703) passively—that is, the sample is applied to the sample splitting zone and distribution of the sample (i.e., displacement of the sample from the sample splitting zone) into each of the twenty fluid channels (703) is achieved via positive pressure applied to the inlet (701) or negative pressure applied via the twenty outlets (706). The challenge for passive distribution is that the volume of sample distributed to each fluid channel (703) must be consistent, since if the sample aliquot volumes are too different the movement of the sample aliquots through the fluid channels (703) and into the first and second reagent wells (704 and 705) cannot be synchronized. In an alternative, the sample may be distributed to the fluid channels (703) actively, e.g., via, e.g., a twenty-way valve, where the sample aliquots are distributed to each fluid channel (703) one at a time, or e.g., two or four at a time. The volume of the sample aliquot distributed into each fluid channel (703) is controlled by the pressure (or vacuum) driving the sample from the sample splitting zone (702) into each fluid channel and the period of time each valve is open. As an additional alternative, a one-way valve could be used to distribute the sample aliquots to each fluid channel (703) by, e.g., rotating the inlet of each fluid distribution channel (703) to match the valve outlet (not shown).

Note that other configurations for the sample splitting zone are possible, including a member or layer above the 2D or parallel module (700) substrate shown in FIG. 7B, where the sample flows from the outside diameter of the 2D or parallel module (700) toward the middle; that is, where outlets (706) are actually inlets and inlet (701) functions as an outlet such that detection takes place in the more compact middle region of 2D or parallel module (700). In addition, although fluid channels (703) are shown here as being straight, the fluid channels (703) between, e.g., the first (704) and second (705) reagent wells may be serpentine shaped to facilitate mixing between the sample and the RNP1s. Other modalities may be employed as well, such as including features in the fluid channels (703) such as constrictions or expansions and/or physical structures such as pillars, creases, steps or fins. Further, the first (704) and/or second (705) reagent wells may comprise features that allow for various mixing strategies, such as bubbling, ultrasonic perturbation, magnetic beads present that may be actuated, or pressure/vacuum can be alternated in a “push/pull” configuration. Other mixing modalities may be employed such as Dean flows in curved channels. Such features induce secondary flows with transverse recirculation.

Whether the sample aliquots are delivered to the fluid channels (703) passively or actively, it is important that the sample aliquots be synchronized as they travel through the fluid channels (703) and as they pass through the first (704) and second (705) reagent wells. In addition to employing active sample aliquot distribution, various other methods can be used to synchronize the sample aliquots. In one method, the first (704) and second (705) reagent wells could be hydrophilic (that is, coated in a bioinert hydrophilic substance) and the outlets of the first (704) and/or second (705) reagent wells could be hydrophobic (that is, coated in a bioinert hydrophobic substance). In this method, the sample aliquot is drawn in to the first (704) and second (705) reagent wells, but movement of the sample aliquot out of the reagent wells is impeded. The action of drawing the sample aliquot into the reagent wells and impeding flow of the sample aliquot out of the first (704) and/or second (705) reagent wells allows sample aliquots that may be moving more slowly to “catch up” with the sample aliquots that are moving more rapidly.

Another exemplary method for synchronizing the movement of the sample aliquots is to constrict the portion of the fluid channels (703) proximate to one or both of the outlets of the first (704) and second (705) reagent wells; that is, to narrow a portion or region of the fluid channels (703) where the fluid channels exit the first and second/or reagent wells (704 and 705). The restriction in flow of the sample aliquots at this (these) “choke point” allows the sample aliquots that may be moving more slowly to “catch up” with the sample aliquots that are moving more rapidly.

A third method for synchronizing the movement of the sample aliquots is to employ gravity to increase the elevation of the flow channels (703) as they progress from the sample splitting zone to the first reagent wells (704) and/or to the second reagent wells (705). As the sample aliquots flow up the fluid channels (703), at some point they will be held in place by a force equal to their weight. Yet another exemplary method for synchronizing the movement of the sample aliquots is to add membranes or fine mesh screens to the flow channels (703). The membranes allow the flow of air but significantly slow the flow of fluid. Membranes also assist in reducing air bubbles and foaming that may occur.

Liquid pinning is an exemplary fourth method that may be used for synchronizing the sample aliquots as they flow through the fluid channels (703) encountering the first reagent well (704) and the second reagent well (705). In one embodiment, liquid pinning uses a physical barrier in the fluid channel (703) to slow the movement of the sample aliquot through the fluid channel (703). For example, the diameter of the flow channel (703) can be restricted by a “step” perpendicular to the direction of the flow of the sample aliquot. The sample aliquot must flow over the step, thereby restricting the velocity of the flow. Liquid pinning can be combined, e.g., with using hydrophobic and hydrophilic surfaces to control the velocity of the flow of the sample aliquots, thereby synchronizing the sample aliquots. Indeed, it should be apparent to one of ordinary skill in the art given the present discussion that one, two or more of these methods may be employed for synchronizing the sample aliquots as they travel through the fluid channels (703).

FIG. 7C illustrates yet another exemplary method for synchronizing and evenly splitting a sample in a 2D module. In this example, a fluid source (e.g., the sample) is connected to the inlet channel. The fluid source is also connected to pneumatic control hardware that can supply positive and negative pressures for fluid control. By pulling a vacuum on the fluid source, the pressure in the downstream channel network is also reduced; however, once the vacuum levels across the system are equilibrated, the pneumatic control hardware on the fluid source reduces the vacuum level, at varying rates as desired (see graphs (i) and (ii)). The reduction in vacuum causes fluid to be pulled from the fluid source and into the downstream channel network, and fluid continues to be drawn into the downstream channel network until the system equilibrates to atmosphere. In addition, positive pressure forces can be applied at the fluid source to send fluid deeper into the downstream channel network. This vacuum filling method is advantageous for applications requiring the even splitting of fluid across a channel network, such as in the present case where even splitting of the sample is desired. In this method, fluid is split according to vacuum levels in the channel network and is significantly less influenced by variations in surface roughness, wettability, etc. Although the initial filling of this system requires channels to initially be dead-ended, a valve system can be used to open the channel ends to allow fluid flow.

As described in relation to FIGS. 1A and 1B, the various assay components may be introduced to the sample, (or the sample introduced to the assay components) in different orders. In some aspects, one or more assay components are introduced into the sample before splitting the sample, and thus are aliquoted along with the sample. In other aspects, all assay components (RNP1s, RNP2s, reporter moieties and blocked nucleic acid molecules) will all reside in the same partition. In yet other aspects, the RNP1s and the blocked nucleic acid molecules may reside in a first partition and the RNP2s and reporter moieties may reside in a partition “downstream” such that the RNP1s are able to react with the sample and unblock the blocked nucleic acid molecules prior to the unblocked nucleic acid molecules encountering the RNP2s and reporter moieties. In short, many scenarios are possible, the key being that the RNP1s reside in separate, known partitions where they are retained.

The diameter “D” of 2D or parallel module (700) will depend on the volume of the sample to be split and the number of target nucleic acids of interest (i.e., RNP1s) to be tested. The assay modules described herein can be designed and optimized for samples from approximately 10 μL to 1 mL in size.

In an alternative embodiment to the “hub and spoke” configuration of the 2D or parallel module (700), a bifurcation configuration could be employed. That is, instead of taking the sample and distributing the sample into twenty channels all at once or one, two or, e.g., four aliquots at a time, the sample could be halved, then halved again, then halved again and so on until the sample is introduced to a desired number of wells. For example, a 500 μL sample could be split into two 250 μL aliquots, then the two 250 μL aliquots are split into four 125 μL aliquots, then the four 125 μL aliquots into eight 62.5 μL aliquots, then the eight 62.5 μL aliquots into sixteen 31 μL aliquots that are distributed into sixteen wells containing RNP1s and some or all of the other assay components.

Again, the embodiment of the 2D or parallel module (700) described above in FIG. 7B and those described in relation to FIGS. 7D-7F, 7G-7J, and 7K-7L below are exemplary only. As stated above, aside from the sample, other assay components with the exception of the RNP1s may be distributed to or may reside (as one or more lyophilized reagents or soluble reagents) in the sample splitting zone. That is, one or more of the reporter moieties, RNP2s, or blocked nucleic acid molecules may be added to the sample splitting zone. In this embodiment, the sample and the one or more assay components would then be distributed into the fluid channels, which would then flow toward a reagent well comprising RNP1 with no need for a second reagent well. In yet another example, no assay components may be distributed via the sample splitting zone; however, there may be only a single reagent well with all assay components—RNP1, RNP2, blocked nucleic acid molecules and reporter moieties—residing in the single reagent well (see, e.g., FIG. 1A, method 120).

FIGS. 7D-7F are illustrations of an “array type” 2D or parallel module (710). FIG. 7D, shows an assembled 2D or parallel module (710) from a top perspective view comprising one to several inlets (701) (or outlets (706) (outlets not shown here)), depending on the configuration of the pumps); here, one in each corner and in the center of the 2D or parallel module (710) originating on a top surface of an upper member (708) of the 2D or parallel module (710), in addition to two inlets (701) (or outlets (706)) from opposing sides of the upper member (708) of the 2D or parallel module (710). As described above, the inlets facilitate fluid movement through the 2D or parallel module (710) via positive or negative pressure. In addition to the upper member (708) of the 2D or parallel module (710), there is a lower member (709). In the embodiment of the 2D or parallel module (710), instead of splitting the sample and distributing the sample aliquots into individual channels that travel to wells—in the “array type” 2D or parallel module (710) the sample is distributed into one or more of the inlets (701) (preferably into the inlet (701) that originates in the top middle of upper member (708) or into two or more of the other inlets (701)) where the sample is distributed into fluid channels (703) which surround and run between wells/partitions (711). The sample will be distributed into and fill wells/partitions (711) where cross-talk between samples is limited by the very low rate of diffusion between wells due to the distance between them.

Although diffusion between partitions/wells is limited, to further minimize cross-talk between wells and to facilitate distribution of the sample into the wells/partitions, the wells may be hydrophilic (e.g., coated with a compound to make the wells hydrophilic) and the interstitial regions surrounding the wells may be hydrophobic (e.g., coated with a compound to make the wells hydrophobic). In another approach, an immiscible fluid (such as an oil) may be distributed across the top of the 2D or parallel module (710) after the sample has been distributed. In yet another method, a physical barrier may be lowered—in some aspects, comprising a grid to sit down on top of the wells and physically separate the wells—or to sweep the excess sample over the top surface of the wells pushing excess fluid away.

Here, the length “L” and width “W” (and depth “D”) of 2D or parallel module (710) will depend on the volume of the sample to be split and the number of target nucleic acids of interest (i.e., RNP1s) to be tested, which dictates the number of wells required. For example, for a, e.g., 100 μL sample split into ten 10 μL aliquots, the length/width of the 2D or parallel module (710) will range from about 0.5 cm to 2 cm, with sample wells having a volume of approximately 5 μL to 15 μL. For a 500 μL sample split into twenty 25 μL aliquots, the length/width of the splitting assay module (710) will range from about 1 cm to 3 cm, with sample wells having a volume of approximately 10 μL to 50 μL. The fluid channels that distribute the sample to the wells have a cross-section of 0.01 cm to 0.1 cm depending on the sample volume and the number of wells/partitions present.

FIG. 7E shows the 2D or parallel module (710) from a top perspective view with upper member (708) transparent such that inlets ((701) or outlets (706)) are seen, as are 25 wells or partitions (711) disposed in lower member (709) (in a 5×5 configuration) and interstitial regions (713) between wells (711). Also seen are reagent solids (lyophilized reagents) or reagents disposed on beads (712) in each of the 25 wells (711). FIG. 7F shows a top perspective view of lower member (709) comprising wells or partitions (711), having lyophilized or airdried reagents (or beads) (712) disposed therein, and including interstitial regions (713) between wells (711). Fluid channels (703) which surround and run between wells/partitions (711) are not formed in lower member (709), but in this embodiment are formed by the fitting of upper member (708) to lower member (709).

As with the 2D or parallel module (710) seen in FIG. 7B, the assay components of the “array type” 2D or parallel module (710) can be distributed in various ways and added in various orders, as long as the RNP1s are contained in different, known partitions. That is, the blocked nucleic acid molecules and the reporter moieties can be added to the sample before splitting and then distributed to each partition/well with the sample aliquots where both the different RNP1s and the RNP2s are distributed into each partition (712). Alternatively, all of RNP2, the blocked nucleic acid molecules and the reporter moieties can be added to the sample before splitting the distributed to the wells or partitions (712); or RNP2 and the reporter moieties can be added to the sample before distribution to wells or partitions (712) with the blocked nucleic acid molecules and RNP1s residing in each well.

FIGS. 7G and 7H are side transparent and side transparent perspective views of an “array type” 2D or parallel module (720) with upper member (708), well member (714), and lower member (709). In each of FIGS. 7F and 7G, inlet (701) and outlet (706) are seen, as are sample splitting zone (702), wells/partitions (711), lyophilized or airdried assay components (712) (or bead-bound assay components (712)), and detection zone (715). Separating well member (714) (and wells/partitions (711)) from lower member (709) is membrane/barrier (716). Membrane (716) may be fluid permeable and/or light permeable and may be impregnated with one or more than one assay components, such as RNP1, RNP2, blocked nucleic acid molecules, and/or reporter moieties. A sample can flow through inlet (701) and sample splitting zone (702) in upper member (708) and on into wells/partitions (711). As with embodiments described above, any one of the assay components except for the RNP1s can be distributed with the sample into wells/partitions (711), including the RNP2s, blocked nucleic acid molecules and reporter moieties. Alternatively, any one of the assay components including the RNP1s can be distributed into wells/partitions (711) as part of the lyophilized or airdried assay components (712) (or bead-bound assay components) or may be impregnated into regions of membrane/barrier (716) including the RNP1s, RNP2s, blocked nucleic acid molecules and/or reporter moieties. In some cases, all assay components aside from the reporter moieties may be included in the lyophilized bead and the reporter moieties are impregnated in membrane (716) at a bottom of each well for imaging.

The membranes (716) useful in the embodiments described herein include those with pore sizes of from 0.02 μm, 0.05 μm, 0.07 μm, 0.10 μm, 0.15 μm, 0.20 μm, 0.25 μm, 0.30 μm, 0.35 μm, 0.40 μm, 0.45 μm, or 0.50 μm, or larger depending on whether beads are used to bind any assay components (in., e.g., a packed bead bed) and what size the beads may be. The membranes (716) may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, and glass fibers. In other embodiments, the membranes may be frangible; i.e., designed to break upon application of a pre-determined pressure.

As with embodiments described above, the length “L” and width “W” (and depth “D”) of 2D or parallel module (720) will depend on the volume of the sample to be split and the number of target nucleic acids of interest (i.e., RNP1s) to be tested. For example, for a, e.g., 100 μL sample split into ten 10 μL aliquots, the length/width of the 2D or parallel module (720) will range from about 0.5 cm to 2 cm, with sample wells having a volume of approximately 5 μL to 15 μL. For a 500 μL sample split into twenty 25 μL aliquots, the length/width of the splitting assay module (720) will range from about 1 cm to 3 cm, with sample wells having a volume of approximately 10 μL to 50 μL. The fluid channels that distribute the sample to the wells have a cross-section of 0.01 cm to 0.1 cm depending on the sample volume and the number of wells/partitions present.

FIG. 7I is a view of detection zone (715) seen through a bottom view of lower member (709). Seen are wells (711). FIG. 7J is a top view of upper member (708) showing fluid channels (703) and wells/partitions (711).

FIGS. 7K and 7L are side transparent and side transparent perspective views, respectively, of an alternative “array type” 2D or parallel module (730) with upper member (708), two well members (714a and 714b), and lower member (709). In each of FIGS. 7K and 7L, inlet (701) and an outlet (706) are seen, as are sample splitting zone (702), upper wells/partitions (711), lower wells/partitions (718), lyophilized or air dried assay components (712) (or bead-bound assay components (712)) in upper wells/partitions (711), lyophilized or air dried assay components (719) (or bead-bound assay components (719)) in lower wells/partitions (718), and detection zone (715). Separating upper well member (714a) and lower well member (714b) (and upper wells/partitions (711) from lower wells/partitions (718)) is membrane (717) and separating lower well member (714b) and lower member (709) is membrane (716).

As described above, membranes (716 and 717) may be fluid permeable and/or light permeable and may be impregnated with or coupled to one or more than one assay component, such as RNP1, RNP2, blocked nucleic acid molecules, and/or reporter moieties. Membrane (717) separating upper well/partition (714a) from lower well/partition (714b) is fluid permeable to allow the sample, RNP1s and other assay components to combine and typically RNP1 will reside in upper well members (711). Membrane (716) separating lower well/partition (714b) from lower member (709) preferably is light permeable to allow imaging in detection zone (715).

A sample can flow through inlet (701) into sample splitting zone (702) in upper member (708), and on into wells/partitions (711) in which the RNP1s reside. The sample fills the wells/partitions (711) reconstituting lyophilized assay components if present, and reactions with the assay components. As with embodiments described above, any one of the assay components except for the RNP1s may be distributed with the sample into wells/partitions (711), including the RNP2s, blocked nucleic acid molecules and reporter moieties. Alternatively, any one of the assay components (including the RNP1s) can be distributed into wells/partitions (711) as part of the lyophilized or airdried assay components (712) (or bead-bound assay components) or may be impregnated into regions of membrane/barrier (717) including the RNP1s, RNP2s, blocked nucleic acid molecules and/or reporter moieties.

Because there is both an upper and a lower well member (714a and 714b), there are additional options for assay component distribution. For example in some embodiments, all assay components aside from the reporter moieties may be included in the lyophilized reagents or coupled to beads (712) in upper wells (711) and the reporter moieties may then be impregnated in membrane (716) at the bottom of the bottom wells/partitions (718) for imaging; alternatively, some assay components (aside from the RNP1s) may be added to the sample before sample splitting and the rest of the assay components will reside in top wells/partitions (711) with bottom wells/partitions (718) used for imaging only. As described in relation to other assay modules above, other configurations of the wells/partitions (711, 718) may be employed such as configuring the wells/partitions side-to-side as opposed to top-to-bottom.

Again, the membranes (716 and 717) useful in the embodiments described herein include those with pore sizes of from 0.02 μm, 0.05 μm, 0.07 μm, 0.10 μm, 0.15 μm, 0.20 μm, 0.25 μm, 0.30 μm, 0.35 μm, 0.40 μm, 0.45 μm, or 0.50 μm, or larger depending on whether beads are used to bind any assay components and what size the beads may be. Depending on the purpose of the membranes (716 and 717) (e.g., delivery of impregnated assay components, retention of beads, allowing fluids to pass through the membrane once wetted), they may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, and glass fibers.

As with embodiments described above, the length “L” and width “W” (and depth “D”) of 2D or parallel module (730) will depend on the volume of the sample to be split and the number of target nucleic acids of interest (i.e., RNP1s) to be tested. For example, for a, e.g., 100 μL sample split into ten 10 μL aliquots, the length/width of the 2D or parallel module (730) will range from about 0.5 cm to 2 cm, with sample wells having a volume of approximately 5 μL to 15 μL. For a 500 μL sample split into twenty 25 μL aliquots, the length/width of the splitting assay module (730) will range from about 1 cm to 3 cm, with sample wells having a volume of approximately 10 μL to 50 μL. The fluid channels that distribute the sample to the wells have a cross-section of 0.01 cm to 0.1 cm depending on the sample volume and the number of wells/partitions present.

FIG. 7M is an overview of the principles behind one sample splitting method. Because the present signal boost assay methods do not involve amplification of the nucleic acids from source organisms, other approaches may be used to increase assay sensitivity. One sample splitting method includes the steps of shearing or fragmenting the nucleic acids obtained from the genomes of the source organisms in a sample, then designing and using several to many first guide nucleic acids (gRNA1s) specific for different loci from each source organism of interest.

At right in FIG. 7M are seen four exemplary source organisms, e.g., source organism X (with genome 752), source organism Y (with genome 762), source organism Z (with genome 772), and source organism AA (with genome 782). Each genome (752, 762, 772, and 782) of each source organism (X, Y, Z and AA, respectively) comprises four genomic loci (753, 754, 755, and 756; 763, 764, 765, and 766; 773, 774, 775, and 776; and 783, 784, 785 and 786, respectively) that will be interrogated by the first ribonucleoprotein complexes (RNP1s). That is, the first ribonucleoprotein complexes (RNP1s) comprise four different first guide nucleic acids (gRNA1s) for each of the four source organisms; thus, in this example, there will be sixteen different RNP1s, four for each source organism.

During sample preparation, the source organisms are lysed and the nucleic acids from the source organisms are fragmented, resulting in fragments (757) from genome (752) from source organism X, fragments (767) from genome (762) from source organism Y, and fragments (777) from genome (772) from source organism Z; however, for the purpose of demonstration of the principle of splitting and multiplexing, imagine genome (782) of source organism AA is not fragmented. Lysing and fragmentation results in a pool of fragmented nucleic acids from the sample except for source organism AA, where the genomic nucleic acids 782 remain unfragmented. In a next step, the sample is split and aliquots (790, 791, 792, and 793) are distributed into partitions (794, 795, 796, and 797). Partition (794) comprises gRNA1s (753′, 754′, 755′, and 756′) specific for fragments (753, 754, 755, and 756) from genome (752) of source organism X; partition (795) comprises gRNA1s (763′, 764′, 765′, and 766′) specific for fragments (763, 764, 765, and 766) from genome (762) of source organism Y; partition (796) comprises gRNA1s (773′, 774′, 775′, and 776′) specific for fragments (773, 774, 775, and 776) from genome (772) of source organism Z; and partition (797) comprises gRNA1s (783′, 784′, 785′, and 786′) specific for fragments (783, 784, 785, and 786) from genome (782) of source organism AA.

Aliquot (790) is distributed into partition (794). Because aliquot (790) comprises fragment (753) from genome (752), source organism X is detected by gRNA1 (753′) in partition (794). Aliquot (791) is distributed into partition (795). Because aliquot (791) comprises fragment (763) from genome (762), source organism Y is detected by gRNA1 (763′) in partition (795). Aliquot (792) is distributed into partition (796). Because aliquot (792) comprises fragment (774) from genome (772), source organism Z is detected by gRNA1 (774′) in partition (796). Aliquot (793) is distributed into partition (797). Aliquot (793) does not comprise a fragment that corresponds to genome (782) of source organism AA and thus source organism AA is not detected by any gRNA1 (783′, 784′, 785′, or 786′) in partition (797).

Source organism (782) was not fragmented, therefore all genomic loci (783, 784, 785 and 786) remained on the genome (782) of source organism AA, which was in aliquot (790) distributed into partition (794) comprising the gRNA1s for source organism X. Note that because the genome (782) for source organism AA was not fragmented, source organism AA would have only been detected if aliquot (790) had been distributed into partition (797). Note that a sample will contain several to many genome copies from the source organisms in the sample depending on the prevalence of a particular source organism in the sample; therefore, there will be several to many copies of each fragment from these genomes.

### 3D or Array Modules

FIG. 8A is a graphic representation of the 3D or array module. Seen in FIG. 8A is an RNP1 array (800) comprising partitions (802-831) on substrate (801). Partitions (802-831) may all comprise different RNP1s (RNP1s that differ on the basis of at least the gRNA1 present) or some or all of the RNP1s may be disposed in, e.g., duplicate or triplicate partitions. That is, partitions (802-831) may all comprise different gRNA1s or some partitions (e.g., 802-804 and 822-824) may comprise the same gRNA1 and other partitions (e.g., 805-821, 823, and 825-831) may comprise different gRNAs from one another and from partitions (802-804 and 822-824). As described above in relation to FIG. 7A, in preferred embodiments the RNP1s would be lyophilized or air dried, or, in some embodiments, the RNP1s may reside with one or more assay components on a functionalized bead or coupled to the surface of the assay module substrate.

Sample (840) comprising nucleic acids (841-849) is flowed over or otherwise introduced to the RNP1 3D or array module under conditions that enable target nucleic acids of interest, if present, to bind to complementary gRNA1s in the RNP1s. Of the nucleic acids (841-849) only one nucleic acid (e.g., 843) may be a target nucleic acid of interest. Finally, a reaction mixture of assay components 840 comprising RNP2s, blocked nucleic acid molecules (or in other embodiments described above, blocked primer molecules or blocked guide molecules) and reporter molecules are introduced to the RNP1 array under conditions that enable the signal boost assay to take place. Once the signal boost assay takes place, reporter signals, if present, are detected and the target nucleic acids of interest present in the sample are identified, as seen at partition (818).

FIGS. 8B-8D are different perspective views and exemplary embodiments of a 3D or array module. FIG. 8B is a top view of a 3D or array module (860). 3D or array module (860) comprises a top surface (861), an assay region (862), where the assay region (862) comprises multiple partitions or wells (863) (here, in a 5×5 configuration), interstitial regions (867), an inlet (864), and an outlet (865). In this embodiment, the sample is not split, but is delivered to the 3D or array module (860) via inlet (864), which addresses all wells via fluid channels (866). In this embodiment, wells (863) comprise RNP1s at known addresses, but may also comprise some or all of the other assay components (i.e., RNP2s, blocked nucleic acids, and reporter moieties). For example, if wells (863) comprise all assay components, the sample is delivered to wells (863) in bulk and then imaging can occur. In other embodiments, the wells may comprise both RNP1s and RNP2s, the sample is delivered to wells (863) in bulk, and then the blocked nucleic acid molecules and reporter moieties are delivered to the wells (863) in bulk. In some embodiments, the RNPs (and one or more other assay components, if applicable) are coupled to the bottom of the wells or are coupled to beads of size 0.1-100 microns, or 1-100 microns or 1-10 microns in size to optimize retention of the RNP1s (and one or more other assay components, if applicable) in the proper well. Bead sizes appropriate for the present assay modules are those that are large enough such that diffusion throughout the assay module cannot take place.

If other assay components (aside from RNP1) are delivered in bulk, in some embodiments any excess reaction mix following delivery to wells (863) is removed or “swept” from the top of assay region (862) using oil or a physical barrier such as a membrane that layers on the top of assay region (862) “pushing” the excess reaction mix from the end of the assay region (862) with inlet (864) to the end of the assay region (862) with outlet (865). As the sample is delivered in bulk, in some embodiments active mixing of the sample may be employed, including magnetic beads or bubbling or other methods (e.g., push/pull strategies, ultrasonic mixing) for moving the sample throughout wells (863) in assay region (862). Alternatively or in addition, other modalities for mixing may be employed as well, such as including features in the fluid channels such as constrictions or expansions and/or physical structures such as pillars, creases, steps or fins, or pressure/vacuum can be alternated in a “push/pull” configuration. Other mixing modalities may be employed such as Dean flows in curved channels. Such features induce secondary flows with transverse recirculation. Preferably, the RNP1s will be coupled to the surface of the partitions or retained on beads if active mixing of the sample is to be employed. Note that although wells (863) are presented here in a 5×5 configuration, other configurations (e.g., 3×4, 2×10, concentric circles, etc.) are contemplated.

FIG. 8C is a side transparent perspective view of an alternative 3D or array module (870) comprising a “double layer” wherein partitions or wells (863) are positioned under partitions or wells (873). Seen in this view of 3D or array module (870) are top surface (861), inlet (864), fluid channels (866), and top (882) of 3D or array module (870).

FIG. 8D is a cross sectional view of 3D or array module (870) showing wells (863) in layer (884) positioned under wells (873) in layer (883). Wells (873) comprise lyophilized RNP1 (874) and one or more of the other assay components and wells (863) comprise solubilized or lyophilized assay components (875) that are not contained in wells (873). A membrane member (880) separates wells (873) from wells (863). The sample is delivered in bulk to wells (873) comprising RNP1 and, e.g., RNP2 and the reporter moieties and allowed to react with the RNP1s. Following delivery of the sample to the lyophilized RNP1s, RNP2s and reporter moieties in wells (873), membrane (880) is broken via pressure (i.e., a “frangible” barrier or membrane) or dissolved and the contents (874) of wells (873) (e.g., all assay components aside from the blocked nucleic acids (875)) is delivered to wells (863). Once the assay components (874) from wells (873) are delivered to the lyophilized or air dried blocked nucleic acids (875) in wells (863), the assay reaction takes place and imaging can then be performed through, e.g., the bottom of wells (863).

Membranes appropriate for use in the “double layer” 3D or array module are those that are biocompatible and able to retain the assay components in either a solubilized state or a lyophilized state and/or coupled to beads and provided, e.g., in a packed bed of beads, whichever is employed. For example, membranes with pore sizes from 0.02 μm, 0.05 μm, 0.07 μm, 0.10 μm, 0.15 μm, 0.20 μm, 0.25 μm, 0.30 μm, 0.35 μm, 0.40 μm, 0.45 μm, or 0.50 μm may be used. As described above, the membranes may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber. The membranes may be hydrophobic and are frangible or burstable, predictably broken upon application of a pre-determined positive or negative pressure. In an alternative embodiment, the membrane may be soluble where the assay components in wells (873) are solids (i.e., lyophilized or air dried); however, delivery of the sample to wells (873) solubilizes the solid assay components, which in turn solubilize the membrane allowing delivery of the now solubilized assay components to the RNP1s (and other assay components, if present) into wells 863. Although this embodiment has wells (863) and (873) configured such that RNP1 is in a bottom well (863) and other assay components are in a top well (873), the double layer array may be configured such that that RNP1 is in a top well (873) and other assay components are in a bottom well (863), or wells (863) and (873) may be configured side-by-side.

The length “L” and width “W” (and depth “D”) of 3D or array module 870 will depend on the volume of the sample and the number of target nucleic acids of interest (i.e., RNP1s) to be tested. For example, for a, e.g., 100 μL sample split into ten 10 μL aliquots, the length/width of the 2D or parallel module (870) will range from about 0.5 cm to 2 cm, with sample wells having a volume of approximately 5 μL to 15 μL. For a 500 μL sample split into twenty 25 μL aliquots, the length/width of the splitting assay module (870) will range from about 1 cm to 3 cm, with sample wells having a volume of approximately 10 μL to 50 μL. The fluid channels that distribute the sample to the wells have a cross-section of 0.01 cm to 0.1 cm depending on the sample volume and the number of wells/partitions present.

FIGS. 8E-8H illustrates alternative 3D or array module embodiments, where again instead of splitting the sample, the sample is distributed in bulk to the 3D or array module. In the embodiment in FIGS. 8E-8H, instead of a grid array as shown in FIGS. 8B-8D, the sample is distributed through a fluid channel sequentially to the various RNP1s spaced along fluid channels. FIG. 8E illustrates a top-down transparent view of an array module (850) in a circular configuration, comprising twelve wells (853) connected by fluid channels (856). Fluid inlet (854) and outlet (855) are shown. In this configuration, known RNP1s reside in the wells (853) and the sample and other reaction mix components are flowed sequentially through fluid channels (856). For sequential delivery, it is important that the RNP1s are immobilized and cannot move from one well to another, which would lead to faulty results. Further, it is important that the sample is allowed adequate time to interact with the RNP1s in each well. Finally, once the sample has interacted with each RNP1, delivery of the other reaction mix components (i.e., RNP2, blocked nucleic acid molecules, reporter moieties) optimally is employed such that the reaction mix components cannot be activated by RNP1 trans-cleavage activity, which runs the risk of transferring activated reporter moieties from one well to the next.

For sequential bulk delivery, thus, the sample is delivered to assay module (850) via inlet (854) and moves through the fluid channels (856) from the first well (853), to the second well (853), to the third well, and so on. The sample may be flowed at a constant slow rate or the sample may be flowed and arrested at intervals to assure that each RNP1 is allowed to interact adequately with the sample. Flow rates can vary depending, e.g., on sample size and the number of wells; however, typical flow rates are between 10-1000 μL/minute, or between 50-500 μL/minute, or between 100-250 μL/minute. After the sample has flowed through assay module (850), the remaining reaction mix components (again, i.e., RNP2, blocked nucleic acid molecules, reporter moieties) are flowed through fluid channels (856); however, because cross-contamination is an issue in sequential delivery, it is important that the reaction mix components not be activated by RNP1 trans-cleavage activity, where the activated RNP2s or reporter moieties could then be transferred from one well to the next. One approach to preventing initial activation—but that allows for subsequent activation—is to lower the temperature of the assay module (850) and reaction mix components until the reaction mix components have been distributed to all wells (853). Once distribution is complete, the temperature of the assay module (850) can be raised so that if a target nucleic acid of interest is present in a well, RNP1 trans-cleavage activity is triggered and the cascade occurs. As described above, the sample and reaction mix is driven by air displacement (using positive or negative pressure) through the fluid channels (856); further, valves may be used in this embodiment as well.

FIG. 8F illustrates a 3D or array module (876) similar to array module (850) in FIG. 8E viewed from the top (861) comprises a fluid inlet (864) and outlet (865), with fluid channels (866) flowing counterclockwise around 3D or array module (876); however, array module (876) comprises a three-dimensional channel structure where the fluid channels (866) flow through both top member (883) and bottom member (885) and traverse vertically in addition to horizontally. Outlet (865) resides on the bottom (not labeled) of bottom member (884). Also seen are “blisters” (869) adjacent to fluid channels (866), residing both on the top (861) of top member (883) and the bottom of bottom member (885). Blisters (869) comprise some or all assay reagents aside from the RNP1s and, when triggered, can dispense the assay reagents into the fluid channels (866). FIG. 8G shows inlet (864) and outlet (865), with fluid channels (866) around the top (861) of top member (883) and bottom of bottom member (885) (fluid channels in bottom member (885) not seen) of 3D or array module (876). A blister (869), fluid channel (866) and RNP1 region is shown (877) and described in more detail in relation to FIG. 8H.

FIG. 8H shows fluid channels (866), a blister (869), and membrane (880). Here, fluid channels (866) are configured horizontally, connected by a vertical region (877) comprising two compartments, lower RNP1 region (878) and upper RNP1 region (879), separated by membrane (880). Membrane (880) may comprise RNP1s (and one or more other assay components) impregnated into membrane (880) or the RNP1s (and one or more other assay components) may reside on functionalized beads (for example, in a packed bed of beads) retained by membrane (880). If not residing on beads, the RNP1s (and other assay components, if present) may be impregnated on either side or both sides of membrane (880). In use, the sample flows horizontally through the fluid channels (866), vertically through vertical region (877) and membrane (880) impregnated with the RNP1s, where target nucleic acids of interest, if present, can bind to the RNP1s. The sample continues to flow through the vertical region (877) of the fluid channel (866), then into the horizontal portion of fluid channel (866) and on to the next vertical region comprising the next RNP1. In this method, the target nucleic acids of interest are captured by the RNP1s as the sample flows through the fluid channels (866) encountering all RNP1s sequentially. As described above, the sample is driven by air displacement (using positive or negative pressure) through the fluid channels (866).

The blisters (869) (one is shown in FIG. 8H) are fluidically connected to the fluid channels (866) at the vertical regions (877) and comprise one or more assay components aside from the RNP1s. Once the sample has passed through fluid channels (866), vertical regions (877) and the series of membranes (880) comprising the different RNP1s, the target nucleic acids of interest, if present, have been captured by the appropriate RNP1s and the remaining assay components can be delivered from the blisters (869) to the vertical regions (877) and membrane (880) by rupturing a membrane or barrier (not shown) separating the contents of the blister (869) and the vertical region (877) of the fluid channel (866). As described above, the assay components may be provided in virtually any sequence as long as the RNP1s are provided at partitioned, known locations in the 3D or array module. Here, the RNP1s may be impregnated into membranes (880) disposed in the vertical regions (877) or disposed on functionalized beads (in a, e.g., packed bead bed) retained by the membranes (880). In addition to the RNP1s, the RNP2s may be impregnated into membranes (880) or retained on functionalized beads as well, or may be sequestered along with, e.g., the blocked nucleic acid molecules and/or reporter moieties in the blisters (869). In this embodiment, the blisters (869) are shown on both the top and the bottom of 3D or array module (876) but may in other embodiments reside on only the top or bottom of 3D or array module (876).

FIGS. 8I and 8J illustrate yet another embodiment of a 3D or array module embodiment (890), where again, instead of splitting the sample, the sample is distributed in bulk to the 3D or array module. In the embodiment in FIGS. 8I-8J, instead of a grid array as shown in FIGS. 8B-8D and like that shown in FIGS. 8E-8H, the sample is distributed through a fluid channel (891) sequentially to the various RNP1s residing in reaction regions (894). The sample flows through fluid channels (891) encountering the different RNP1s residing in (i.e., functionally attached or coupled in or residing on functionalized beads) reaction regions (894). As described above, in addition to the different RNP1s, one or more other assay components may reside in reaction regions (894) as well, including RNP2s and/or reporter moieties.

In use, the sample (895) flows horizontally through the sample channels (891) and reagent regions (894) comprising the RNP1s. The target nucleic acid(s) of interest, if present, can then bind to the RNP1s. The sample continues to flow through the fluid channels (891) and on to the reaction region (894) comprising the next RNP1. In this method as with the 3D or array module described in relation to FIGS. 8E-8H, the target nucleic acids of interest are captured by the RNP1s as the sample flows through the fluid channels (891) encountering all RNP1s sequentially. As described above, the sample is driven by air displacement (using positive or negative pressure) through the fluid channels (866).

Optional one-way valves (not shown) residing in valve regions (893) are opened using the pressure driving the sample through the 3D or array module, then closed once the pressure is relieved, isolating each reaction region (894) from other reaction regions (864) and thus each RNP1 from other RNP1s. The assay components (896) that are not residing in reaction regions (894) (e.g., such as the blocked nucleic acid molecules and reporter moieties) can then be delivered via reagent channels (892) that are oriented perpendicularly to the sample channels (891) to the reaction regions (894) and detected in detection regions (897) downstream of reaction regions (894).

The length “L” and width “W” (and depth “D”) of 3D or array module (890) will depend on the volume of the sample and the number of target nucleic acids of interest (i.e., RNP1s) to be tested. For example, for a, e.g., 100 μL sample split into ten 10 μL aliquots, the length/width of the 2D or parallel module (890) will range from about 0.5 cm to 2 cm, with sample wells having a volume of approximately 5 μL to 15 μL. For a 500 μL sample split into twenty 25 μL aliquots, the length/width of the splitting assay module (890) will range from about 1 cm to 3 cm, with sample wells having a volume of approximately 10 μL to 50 μL. The fluid channels that distribute the sample to the wells have a cross-section of 0.01 cm to 0.1 cm depending on the sample volume and the number of wells/partitions present.

### The Assay Array Modules in an Assay System

The assay modules described herein may be one module in a two- to many-module system, which may include a sample prep module, as well as a detection and imaging module. For example, the sample prep module may comprise various modalities for lysing cells, extracting nucleic acids from cells and purifying the nucleic acids. In one example, the sample prep module may utilize bead beating methods and devices. Bead beating is a known effective method of cell lysis which is used to disrupt virtually any biological sample by agitating the sample (and thus the source organisms) with a lysing matrix (i.e., a grinding medium or beads) in a bead beater instrument. The lysing matrix refers to the physical beads or matrix used to lyse and homogenize the samples and the lysing matrix chosen depends on the size and physical properties of the source organisms (e.g., whether there is a cell wall, if the source organism is a spore). Bead shape determines how cells are disrupted and influences how aggressive the lysing process is. In addition to cell lysis, bead beating can shear nucleic acids into fragments to improve sensitivity for array-based or partitioned reaction detection formats. Lysing beads are classified as spherical, utilizing impaction as the leading force, and angular, generating mechanical shear forces to chop and cut samples. The bead size selected is based on the properties of the material being disrupted and the compatibility with the device hardware, fluidics and processing steps. Finally, beads are fashioned from many different materials, which vary depending on desired hardness, durability and chemical resistance including but not limited to glass, zirconium silicate, zirconium oxide, stainless steel, silica, various ceramics, and silicon carbide. For example, bacteria, and algae comprise a soft cell wall and small cell size and lysing matrices that are spherical and small made of silica, ceramics or glass are appropriate; whereas yeast, fungi, and environmental samples such as soil and wood have harder cells walls and larger cell size and lysing matrices that deliver medium sheer and high impact such as silicon carbide or zirconium oxide are appropriate.

In the present protocols, 100 μm glass or zirconia-silicate beads (BioSpec, Bartelsville, OK USA) are used and a custom bead beater instrument was utilized. Alternatively, commercially available bead beating systems are known and include the Biospec BeadBeater, Powerlyzer 24 Homognizer, the Fisherbrand™ Bead Mill, the Bead Bug™ microtube homogenizer, and the suite of MP FastPrep™ Instruments.

In addition to a sample prep module, the assay modules described herein may be included with a detection and imaging module capable of detecting the presence of the reporter moieties-typically fluorescent reporter moieties-used in the signal boost assays. For example, the optical measurement devices may include a microscope (e.g., a digital microscope), a spectrophotometer, a photometer, a spectrometer, a camera, a spectral camera, a hyperspectral camera, a fluorometer, a spectrofluorometer, and/or a photodetector (such as a photodiode, a photoresistor, and/or a phototransistor). For some applications, the detection and imaging module includes dedicated light sources (such as light emitting diodes, incandescent light sources, etc.) and/or optical elements for manipulating light collection and/or light emission (such as lenses, diffusers, filters, etc.). Typically, the detection and imaging module comprises a set of fluorescent light sources (e.g., light emitting diodes) that are configured to be used for fluorescent imaging. Exemplary image-capturing devices include, but are not limited to, a camera (e.g., a charge-coupled device (CCD) camera or a scientific complementary metal oxide semiconductor sensor (CMOS or sCMOS) camera. Fluorescence intensity is proportional to the amount of unquenched reporter moieties, thus enabling quantitative measurements.

A computer processor typically receives and processes optical measurements that are performed by the detection and imaging module. The computer processor controls the acquisition of optical images that are performed by the detection and imaging module. The computer processor typically communicates with a memory, comprises a user interface where a user may send instructions to the computer processor, and generates an output via an output device including a display.

### Uses of the Signal Boost Assays and Assay Array Modules

As described above, the present disclosure describes signal boost assays for detecting a target nucleic acid of interest in a sample that provide instantaneous or nearly instantaneous results in less than ten minutes including sample prep, allow for massive multiplexing and minimum workflow yet provide accurate results at low cost. Moreover, the various embodiments of the signal boost assay are notable in that, with the exception of the gRNAs in RNP1, the signal boost assay components may stay the same no matter what target nucleic acid(s) of interest are being detected and RNP1 is easily reprogrammed using known guide design tools. As described above, the signal boost assay can be massively multiplexed for detecting several to many to target nucleic acid molecules simultaneously without amplification of the nucleic acids in the sample. For example, the assay may be designed to detect several to many different pathogens (e.g., testing for many different pathogens in one assay), or the assay may be designed to detect one to several to many different sequences from the same pathogen (e.g., to increase specificity and sensitivity), or a combination of the two.

As described above, early and accurate identification of, e.g., infectious agents, microbe contamination, and variant nucleic acid sequences that indicate the present of such diseases such as cancer or contamination by heterologous sources is important in order to select correct therapeutic treatment, identify tainted food, pharmaceuticals, cosmetics and other commercial goods; and to monitor the environment. The signal boost assay described herein can be applied in diagnostics for, e.g., infectious disease (including but not limited to Covid, HIV, flu, the common cold, Lyme disease, STDs, chicken pox, diptheria, mononucleosis, hepatitis, UTIs, pneumonia, tetanus, rabies, malaria, dengue fever, Ebola, plague), for rapid liquid biopsies and companion diagnostics (biomarkers for cancers, early detection, progression, monitoring), prenatal testing (including but not limited to chromosomal abnormalities and genetic diseases such as sickle cell, including over-the-counter versions of prenatal testing assays), rare disease testing (achondroplasia, Addison's disease, α1-antitrypsin deficiency, multiple sclerosis, muscular dystrophy, cystic fibrosis, blood factor deficiencies), SNP detection/DNA profiling/epigenetics, genotyping, low abundance transcript detection, labeling for cell or droplet sorting, in situ nucleic acid detection, sample prep, library quantification of NGS, screening biologics (including engineered therapeutic cells for genetic integrity and/or contamination), development of agricultural products, food compliance testing and quality control (e.g., detection of genetically modified products, confirmation of source for high value commodities, contamination detection), infectious disease in livestock, infectious disease in cash crops, livestock breeding, drug screening, personal genome testing including clinical trial stratification, personalized medicine, nutrigenomics, drug development and drug therapy efficacy, transplant compatibility and monitoring, environmental testing and forensics, and bioterrorism agent monitoring.

Target nucleic acids of interest are derived from samples as described in more detail above. Suitable samples for testing include, but are not limited to, any environmental sample, such as air, water, soil, surface, food, clinical sites and products, industrial sites and products, pharmaceuticals, medical devices, nutraceuticals, cosmetics, personal care products, agricultural equipment and sites, and commercial samples, and any biological sample obtained from an organism or a part thereof, such as a plant, animal, or microbe. In some embodiments, the biological sample is obtained from an animal subject, such as a human subject. A biological sample may be any solid or fluid sample obtained from, excreted by or secreted by any living organism, including, without limitation, single celled organisms, such as bacteria, yeast, protozoans, and amoebas among others, multicellular organisms including plants or animals, including samples from a healthy or apparently healthy human subject or a human patient affected by a condition or disease to be diagnosed or investigated, such as an infection with a pathogenic microorganism, such as a pathogenic bacteria or virus.

For example, a biological sample can be a biological fluid obtained from a human or non-human (e.g., livestock, pets, wildlife) animal, and may include but is not limited to blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface (e.g., a nasal or buccal swab).

The sample can be a viral or bacterial sample or a biological sample that has been minimally processed as described herein, e.g., only treated with a brief lysis step prior to detection. In other embodiments, minimal processing can include thermal lysis at an elevated temperature. In some embodiments, minimal processing can include treating the sample with chaotropic salts such as guanidine isothiocyanate or guanidine HCl and in some embodiments, minimal processing may include contacting the sample with reducing agents such as DTT or TCEP and EDTA to inactivate inhibitors and/or other nucleases present in the samples. In other embodiments, minimal processing for biofluids may include centrifuging the samples to obtain cell-debris free supernatant before applying the reagents.

In an embodiment where blocked nucleic acid molecules are employed, the components of the signal boost assay may be provided in various kits for testing at, e.g., point of care facilities, in the field, pandemic testing sites, and the like. In one aspect, the kit for detecting target nucleic acids of interest in a sample includes: one or more assay modules, preferably pre-loaded with assay components, where the RNP1s are separated into partitions with, in some embodiments, one or more or all of the additional assay components, including the RNP2s, blocked nucleic acid molecules, and reporter moieties.

In an embodiment where blocked primer molecules are employed, the kit for detecting a target nucleic acid of interest in sample includes: one or more assay modules, preferably pre-loaded with assay components, where the RNP1s are separated into partitions with, in some embodiments, one or more or all of the additional assay components, including the RNP2s, template molecules, blocked primer molecules, a polymerase, NTPs, and reporter moieties.

In an embodiment where blocked guides are involved, the kit for detecting target nucleic acid molecules in sample includes: one or more assay modules, preferably pre-loaded with assay components, where the RNP1s are separated into partitions with, in some embodiments, one or more of all the additional assay components, including second nucleic acid nucleases, blocked guide molecules, RNP2 activator nucleic acids, and reporter moieties. Again, the first gRNAs include a sequence complementary to the target nucleic acids of interest and where binding of RNP1 to one or more of the target nucleic acids of interest activates trans-cleavage activity of the first nucleic acid-guided nuclease.

Any of the kits described herein may further include a sample collection device, e.g., a syringe, lancet, nasal swab, or buccal swab for collecting a biological sample from a subject, and/or a sample preparation reagent, e.g., a lysis reagent. Each component of the kit may be in a separate container or two or more components may be in the same container although the RNP1s will be partitioned. In addition, the kit may further include instructions for use and other information.

### EXAMPLES

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Preparation of Sample Nucleic Acids

Mechanical lysis: Nucleic acids of interest may be isolated by various methods depending on the cell type and source (e.g., tissue, blood, saliva, environmental sample, etc.). Mechanical lysis is a widely used cell lysis method and may be used to extract nucleic acids from bacterial, yeast, plant and mammalian cells. Cells are disrupted by agitating a cell suspension with “beads” at high speeds (beads for disrupting various types of cells can be sourced from, e.g., OPS Diagnostics (Lebanon NJ, US) and MP Biomedicals (Irvine, CA, USA)). Mechanical lysis via beads begins with harvesting cells in a tissue or liquid, where the cells are first centrifuged and pelleted. The supernatant is removed and replaced with a buffer containing detergents as well as lysozyme and protease. The cell suspension is mixed to promote breakdown of the proteins in the cells and the cell suspension then is combined with small beads (e.g., glass, steel, or ceramic beads) that are mixed (e.g., vortexed) with the cell suspension at high speeds. The beads collide with the cells, breaking open the cell membrane with shear forces. After “bead beating”, the cell suspension is centrifuged to pellet the cellular debris and beads, and the supernatant may be purified via a nucleic acid binding column (such as the MagMAX™ Viral/Pathogen Nucleic Acid Isolation Kit from ThermoFisher (Waltham, MA, USA) and others from Qiagen (Hilden, Germany), TakaraBio (San Jose, CA, USA), and Biocomma (Shenzen, China)) to collect the nucleic acids (see the discussion of solid phase extraction below).

Solid phase extraction (SPE): Another method for capturing nucleic acids is through solid phase extraction. SPE involves a liquid and stationary phase, which selectively separates the target analyte (here, nucleic acids) from the liquid in which the cells are suspended based on specific hydrophobic, polar, and/or ionic properties of the target analyte in the liquid and the stationary solid matrix. Silica binding columns and their derivatives are the most commonly used SPE techniques, having a high binding affinity for DNA under alkaline conditions and increased salt concentration; thus, a highly alkaline and concentrated salt buffer is used. The nucleic acid sample is centrifuged through a column with a highly porous and high surface area silica matrix, where binding occurs via the affinity between negatively charged nucleic acids and positively charged silica material. The nucleic acids bind to the silica matrices, while the other cell components and chemicals pass through the matrix without binding. One or more wash steps typically are performed after the initial sample binding (i.e., the nucleic acids to the matrix), to further purify the bound nucleic acids, removing excess chemicals and cellular components non-specifically bound to the silica matrix. Alternative versions of SPE include reverse SPE and ion exchange SPE, and use of glass particles, cellulose matrices, and magnetic beads.

Thermal lysis: Thermal lysis involves heating a sample of mammalian cells, virions, or bacterial cells at high temperatures thereby damaging the cellular membranes by denaturizing the membrane proteins. Denaturizing the membrane proteins results in the release of intracellular DNA. Cells are generally heated above 90° C., however time and temperature may vary depending on sample volume and sample type. Once lysed, typically one or more downstream methods, such as use of nucleic acid binding columns for solid phase extraction as described above, are required to further purify the nucleic acids.

Physical lysis: Common physical lysis methods include sonication and osmotic shock. Sonication involves creating and rupturing of cavities or bubbles to release shockwaves, thereby disintegrating the cellular membranes of the cells. In the sonication process, cells are added into lysis buffer, often containing phenylmethylsulfonyl fluoride, to inhibit proteases. The cell samples are then placed in a water bath and a sonication wand is placed directly into the sample solution. Sonication typically occurs between 20-50 kHz, causing cavities to be formed throughout the solution as a result of the ultrasonic vibrations; subsequent reduction of pressure then causes the collapse of the cavity or bubble resulting in a large amount of mechanical energy being released in the form of a shockwave that propagates through the solution and disintegrates the cellular membrane. The duration of the sonication pulses and number of pulses performed varies depending on cell type and the downstream application. After sonication, the cell suspension typically is centrifuged to pellet the cellular debris and the supernatant containing the nucleic acids may be further purified by solid phase extraction as described above.

Another form of physical lysis is osmotic shock, which is most typically used with mammalian cells. Osmotic shock involves placing cells in DI/distilled water with no salt added. Because the salt concentration is lower in the solution than in the cells, water is forced into the cell causing the cell to burst, thereby rupturing the cellular membrane. The sample is typically purified and extracted by techniques such as e.g., solid phase extraction or other techniques known to those of skill in the art.

Chemical lysis: Chemical lysis involves rupturing cellular and nuclear membranes by disrupting the hydrophobic-hydrophilic interactions in the membrane bilayers via detergents. Salts and buffers (such as, e.g., Tris-HCl pH8) are used to stabilize pH during extraction, and chelating agents (such as ethylenediaminetetraacetic acid (EDTA)) and inhibitors (e.g., Proteinase K) are also added to preserve the integrity of the nucleic acids and protect against degradation. Often, chemical lysis is used with enzymatic disruption methods (see below) for lysing bacterial cell walls. In addition, detergents are used to lyse and break down cellular membranes by solubilizing the lipids and membrane proteins on the surface of cells. The contents of the cells include, in addition to the desired nucleic acids, inner cellular proteins and cellular debris. Enzymes and other inhibitors are added after lysis to inactivate nucleases that may degrade the nucleic acids. Proteinase K is commonly added after lysis, destroying DNase and RNase enzymes capable of degrading the nucleic acids. After treatment with enzymes, the sample is centrifuged, pelleting cellular debris, while the nucleic acids remain in the solution. The nucleic acids may be further purified as described above.

Another form of chemical lysis is the widely used procedure of phenol-chloroform extraction. Phenol-chloroform extraction involves the ability for nucleic acids to remain soluble in an aqueous solution in an acidic environment, while the proteins and cellular debris can be pelleted down via centrifugation. Phenol and chloroform ensure a clear separation of the aqueous and organic (debris) phases. For DNA, a pH of 7-8 is used, and for RNA, a more acidic pH of 4.5 is used.

Enzymatic lysis: Enzymatic disruption methods are commonly combined with other lysis methods such as those described above to disrupt cellular walls (bacteria and plants) and membranes. Enzymes such as lysozyme, lysostaphin, zymolase, and protease are often used in combination with other techniques such as physical and chemical lysis. For example, one can use cellulase to disrupt plant cell walls, lysosomes to disrupt bacterial cell walls and zymolase to disrupt yeast cell walls.

### Example II: RNP Formation

For RNP complex formation, 250 nM of LbCas12a nuclease protein was incubated with 375 nM of a target specific gRNA in 1X Buffer (10 mM Tris-HCl, 100 μg/mL BSA) with 2-15 mM MgCl₂ at 25° C. for 20 minutes. The total reaction volume was 2 μL. Other ratios of LbCas12a nuclease to gRNAs were tested, including 1:1, 1:2 and 1:5. The incubation temperature ranged from 16° C.-37° C., and the incubation time ranged from 10 minutes to 4 hours.

### Example III: Blocked Nucleic Acid Molecule Formation

Ramp cooling: For formation of the secondary structure of blocked nucleic acid molecules, 2.5 μM of a blocked nucleic acid molecule (any of Formulas I-IV) was mixed in a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to 37° C. at 0.015° C./second to form the desired secondary structure.

Snap cooling: For formation of the secondary structure of blocked nucleic acid molecules, 2.5 μM of a blocked nucleic acid molecule (any of Formulas I-IV) was mixed in a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to room temperature by removing the heat source to form the desired secondary structure.

Snap cooling on ice: For formation of the secondary structure of blocked nucleic acid molecules, 2.5 μM of a blocked nucleic acid molecule (any of Formulas I-IV) was mixed in a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to room temperature by placing the reaction tube on ice to form the desired secondary structure.

### Example IV: Reporter Moiety Formation

The reporter moieties used in the reactions herein were single-stranded DNA oligonucleotides 5-9 bases in length (e.g., with sequences of TTATT, TTTATTT, ATTAT, ATTTATTTA, AAAAA, or AAAAAAAAA) with a fluorophore and a quencher attached on the 5′ and 3′ ends, respectively. In one example using a Cas12a cascade, the fluorophore was FAM-6 and the quencher was IOWA BLACK® (Integrated DNA Technologies, Coralville, IA). In another example using a Cas13 cascade, the reporter moieties were single-stranded RNA oligonucleotides 5-10 bases in length (e.g., r(U)n, r(UUAUU)n, r(A)n).

### Example V: Signal Boost Assay

Format I (final reaction mix components added at the same time); RNP1 was assembled using the LbCas12a nuclease and a gRNA for the Methicillin resistant Staphylococcus aureus (MRSA) DNA according to the RNP complex formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule (Formula I-IV) using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. Thereafter, the final reaction was carried out in 1X Buffer, with 500 nM of the ssDNA reporter moiety, 1×ROX dye (Thermo Fisher Scientific, Waltham, MA) for passive reference, 2.5 mM MgCl₂, 4 mM NaCl, 15 nM LbCas12a: 22.5 nM gRNA RNP1, 20 nM LbCas12a: 35 nM gRNA RNP2, and 50 nM blocked nucleic acid molecule (any one of Formula I-IV) in a total volume of 9 μL. 1 μL of MRSA DNA target (with samples having as low as three copies and as many as 30000 copies-see FIGS. 6-14) was added to make a final volume of 10 μL. The final reaction was incubated in a thermocycler at 25° C. with fluorescence measurements taken every 1 minute.

Format II (RNP1 and MRSA target pre-incubated before addition to final reaction mix); RNP1 was assembled using the LbCas12a nuclease and a gRNA for the MRSA DNA according to RNP formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule (Formula I-IV) using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. After dilution, the formed RNP1 was mixed with 1 μL of MRSA DNA target and incubated at 16° C.-37° C. for up to 10 minutes to activate RNP1. The final reaction was carried out in 1× Buffer, with 500 nM of the ssDNA reporter moiety, 1×ROX dye (Thermo Fisher Scientific, Waltham, MA) for passive reference, 2.5 mM MgCl₂, 4 mM NaCl, the pre-incubated and activated RNP1, 20 nM LbCas12a: 35 nM gRNA RNP2, and 50 nM blocked nucleic acid molecule (any one of Formula I-IV) in a total volume of 9 μL. The final reaction was incubated in a thermocycler at 25° C. with fluorescence measurements taken every 1 minute.

Format III (RNP1 and MRSA target pre-incubated before addition to final reaction mix and blocked nucleic acid molecule added to final reaction mix last): RNP1 was assembled using the LbCas12a nuclease and a gRNA for the MRSA DNA according to the RNP complex formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule (Formula I-IV) using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. After dilution, the formed RNP1 was mixed with 1 μL of MRSA DNA target and incubated at 16° C.-37° C. for up to 10 minutes to activate RNP1. The final reaction was carried out in 1× Buffer, with 500 nM of the ssDNA reporter moiety, 1×ROX dye (Thermo Fisher Scientific, Waltham, MA) for passive reference, 2.5 mM MgCl₂, 4 mM NaCl, the pre-incubated and activated RNP1, and 20 nM LbCas12a: 35 nM gRNA RNP2 in a total volume of 9 μL. Once the reaction mix was made, 1 μL (50 nM) blocked nucleic acid molecule (any one of Formula I-IV) was added for a total volume of 10 μL. The final reaction was incubated in a thermocycler at 25° C. with fluorescence measurements taken every 1 minute.

### Example VI: Detection of MRSA and Test Reaction Conditions

To detect the presence of Methicillin resistant Staphylococcus aureus (MRSA) and determine the sensitivity of detection with the signal boost assay, titration experiments with a MRSA DNA target nucleic acid of interest were performed. The MRSA DNA sequence (NCBI Reference Sequence NC: 007793.1) is as follows.

SEQ ID NO: 615:
ATGAAAAAGATAAAAATTGTTCCACTTATTTTAATAGTTGTAGTTGTCGGG
TTTGGTATATATTTTTATGCTTCAAAAGATAAAGAAATTAATAATACTAT
TGATGCAATTGAAGATAAAAATTTCAAACAAGTTTATAAAGATAGCAGTT
ATATTTCTAAAAGCGATAATGGTGAAGTAGAAATGACTGAACGTCCGATA
AAAATATATAATAGTTTAGGCGTTAAAGATATAAACATTCAGGATCGTAA
AATAAAAAAAGTATCTAAAAATAAAAAACGAGTAGATGCTCAATATAAAA
TTAAAACAAACTACGGTAACATTGATCGCAACGTTCAATTTAATTTTGTT
AAAGAAGATGGTATGTGGAAGTTAGATTGGGATCATAGCGTCATTATTCC
AGGAATGCAGAAAGACCAAAGCATACATATTGAAAATTTAAAATCAGAAC
GTGGTAAAATTTTAGACCGAAACAATGTGGAATTGGCCAATACAGGAACA
GCATATGAGATAGGCATCGTTCCAAAGAATGTATCTAAAAAAGATTATAA
AGCAATCGCTAAAGAACTAAGTATTTCTGAAGACTATATCAAACAACAAA
TGGATCAAAATTGGGTACAAGATGATACCTTCGTTCCACTTAAAACCGTT
AAAAAAATGGATGAATATTTAAGTGATTTCGCAAAAAAATTTCATCTTAC
AACTAATGAAACAGAAAGTCGTAACTATCCTCTAGGAAAAGCGACTTCAC
ATCTATTAGGTTATGTTGGTCCCATTAACTCTGAAGAATTAAAACAAAAA
GAATATAAAGGCTATAAAGATGATGCAGTTATTGGTAAAAAGGGACTCGA
AAAACTTTACGATAAAAAGCTCCAACATGAAGATGGCTATCGTGTCACAA
TCGTTGACGATAATAGCAATACAATCGCACATACATTAATAGAGAAAAAG
AAAAAAGATGGCAAAGATATTCAACTAACTATTGATGCTAAAGTTCAAAA
GAGTATTTATAACAACATGAAAAATGATTATGGCTCAGGTACTGCTATCC
ACCCTCAAACAGGTGAATTATTAGCACTTGTAAGCACACCTTCATATGAC
GTCTATCCATTTATGTATGGCATGAGTAACGAAGAATATAATAAATTAAC
CGAAGATAAAAAAGAACCTCTGCTCAACAAGTTCCAGATTACAACTTCAC
CAGGTTCAACTCAAAAAATATTAACAGCAATGATTGGGTTAAATAACAAA
ACATTAGACGATAAAACAAGTTATAAAATCGATGGTAAAGGTTGGCAAAA
AGATAAATCTTGGGGTGGTTACAACGTTACAAGATATGAAGTGGTAAATG
GTAATATCGACTTAAAACAAGCAATAGAATCATCAGATAACATTTTCTTT
GCTAGAGTAGCACTCGAATTAGGCAGTAAGAAATTTGAAAAAGGCATGAA
AAAACTAGGTGTTGGTGAAGATATACCAAGTGATTATCCATTTTATAATG
CTCAAATTTCAAACAAAAATTTAGATAATGAAATATTATTAGCTGATTCA
GGTTACGGACAAGGTGAAATACTGATTAACCCAGTACAGATCCTTTCAAT
CTATAGCGCATTAGAAAATAATGGCAATATTAACGCACCTCACTTATTAA
AAGACACGAAAAACAAAGTTTGGAAGAAAAATATTATTTCCAAAGAAAAT
ATCAATCTATTAACTGATGGTATGCAACAAGTCGTAAATAAAACACATAA
AGAAGATATTTATAGATCTTATGCAAACTTAATTGGCAAATCCGGTACTG
CAGAACTCAAAATGAAACAAGGAGAAACTGGCAGACAAATTGGGTGGTTT
ATATCATATGATAAAGATAATCCAAACATGATGATGGCTATTAATGTTAA
AGATGTACAAGATAAAGGAATGGCTAGCTACAATGCCAAAATCTCAGGTA
AAGTGTATGATGAGCTATATGAGAACGGTAATAAAAAATACGATATAGAT
GAATAA

Briefly, an RNP1 was preassembled with a gRNA sequence designed to target MRSA DNA. Specifically, RNP1 was designed to target a 20 bp region of the mecA gene of MRSA: TGTATGGCATGAGTAACGAA (SEQ ID NO: 616). An RNP2 was preassembled with a gRNA sequence designed to target the unblocked nucleic acid molecule that results from unblocking (i.e., linearizing) blocked nucleic acid molecule. The reaction mix contained the preassembled RNP1, preassembled RNP2, and a blocked nucleic acid molecule, in a buffer (pH of about 8) containing 4 mM MgCl₂ and 101 mM NaCl.

The blocked nucleic acid molecule used herein had a secondary structure free energy value of −5.84 kcal/mol and relatively short self-hybridizing, double-stranded regions of 5 bases and 6 bases. Results were achieved for detection of 3E4 copies, 30 copies, 3 copies and 0 copies of the mecA gene of MRSA (n=3) at 25° C. with varying concentrations of blocked nucleic acid, RNP2 and reporter moiety. When 100 nM blocked nucleic acid molecules, 10 nM RNP2s and 500 nM reporter moieties are used, the ratio of blocked nucleic acid molecules to RNP2s was 10:1. With 3E4 copies, nearly 100% of the reporters were cleaved at t=0 with a signal-to-noise ratio of 28.06 at 0 minutes, a signal-to-noise ratio of 24.23 at 5 minutes, and a signal-to-noise ratio of 21.01 at 10 minutes (data not shown). Additionally, the signal-to-noise ratios for detection with 30 copies of MRSA target was 12.45 at 0 minutes, 14.07 at 5 minutes and 16.16 at 10 minutes; and the signal-to-noise ratios for detection with 3 copies of MRSA target is 1.79 at 0 minutes, 1.64 at 5 minutes and is 2.04 at 10 minutes. Note the measured fluorescence at 0 copies increases only slightly over the 10- and 30-minutes intervals, resulting in a flat negative. A flat negative (the results obtained over the time period for 0 copies) demonstrated that there is very little non-specific or undesired signal generation in the system.

### Example VII: Activity of RNPs Following Air Drying

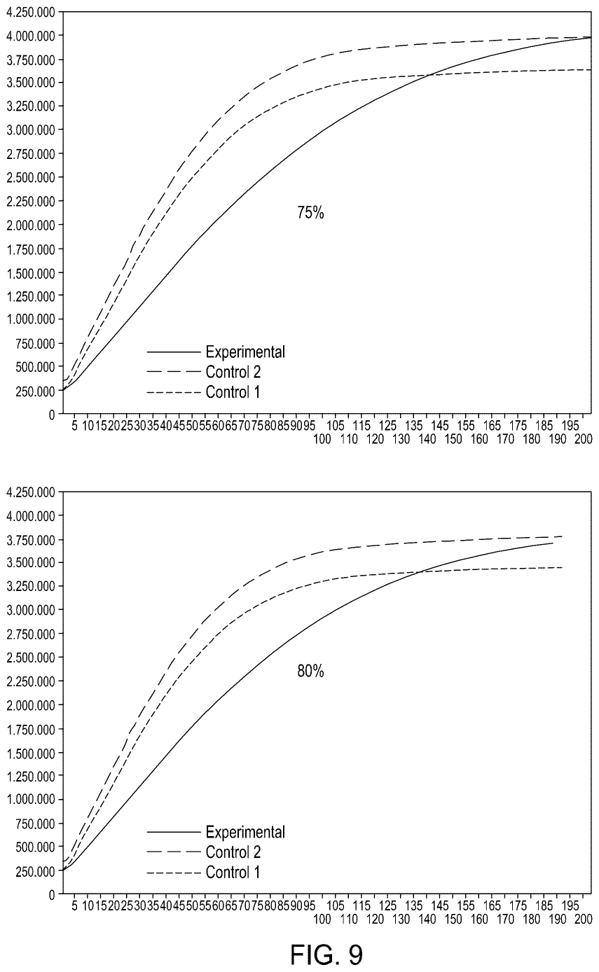

The signal boost assay was performed using air dried RNPs. First, air drying of RNP1s were tested. FIG. 9 shows two graphs of fluorescence units vs. time for replicates where the experimental reaction included 100 nM RNP1 dried for 72 hours at room temperature, with 25 mM Mg, 100 mM Na, 500 mM trehalose pH 7.0; and reconstituted with 10 nM target nucleic acid, and 500 nM reporter. The controls included 1) 10 nM RNP1, 10 nM target, 500 nM reporter, and 50 nM trehalose; and 2) 1) 10 nM RNP1, 10 nM target, and 500 nM reporter (no trehalose). The y-axis is arbitrary fluorescence units and the x-axis is time in minutes. Note activity stood at 75% and 80% for the first and second repetitions.

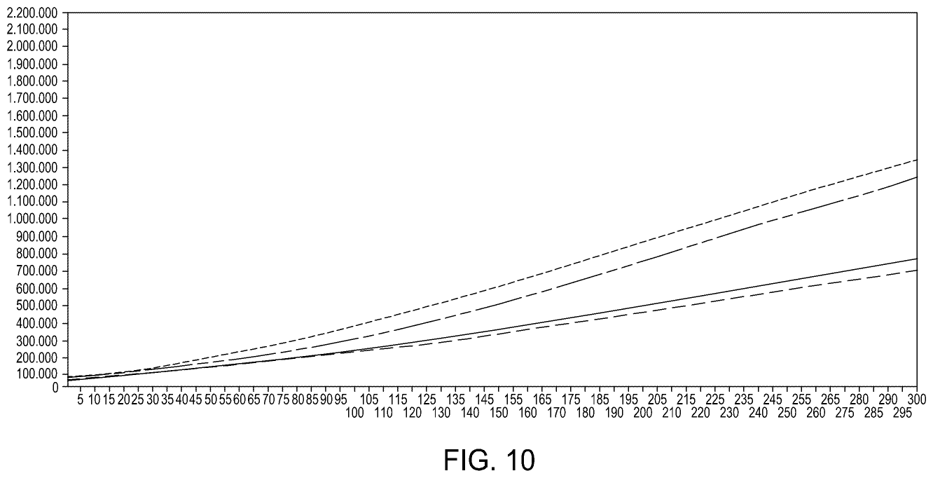

Next, RNP1s and RNP2s were tested after air drying for 72 hours versus no airdrying. FIG. 10 shows a graph of the results obtained using 1) 0 copies of target (control) or 3E4 copies of target (experimental), 20 nM blocked target, 20 nM RNP1, 10 nM RNP2, 500 mM Trehalose, 2.5 mM Mg, 45 mM Na at pH 7.0. The y-axis is arbitrary fluorescence units and the x-axis is time in minutes. Note activity is high even with air drying the RNPs.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

We claim:

1. An assay module for identifying one or more target nucleic acids of interest from two or more source organisms in a sample comprising:
at least one inlet coupled to a sample splitting region, wherein the sample splitting region is fluidically coupled to a plurality of first fluid channels and each first fluid channel is fluidically coupled to a first partition;
a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, and wherein different RNP1s reside in different first partitions;
second ribonucleoprotein complexes (RNP2s) comprising a second nucleic acid-guided nuclease and a second guide nucleic acid complementary to a portion of blocked nucleic acid molecules;
the blocked nucleic acid molecules;
reporter moieties;
a pump configured to provide negative or positive pressure to the assay module; and
a detection and imaging zone.

2. The assay module of claim 1, wherein the RNP1s are lyophilized.

3. The assay module of claim 1, wherein the RNP1s are air dried.

4. The assay module of claim 1, wherein the reporter moieties and blocked nucleic acid molecules are introduced in the sample splitting region with the sample and the RNP2s reside in the first partitions with the RNP1s.

5. The assay module of claim 1, wherein the RNP2s, reporter moieties and blocked nucleic acid molecules are introduced in the sample splitting region with the sample.

6. The assay module of claim 1, comprising valves in the first fluid channels between the sample splitting region and the first partitions.

7. The assay module of claim 1, further comprising second fluid channels connecting the first partitions to second partitions, and wherein the RNP2s, blocked nucleic acid molecules, and reporter moieties reside in the second partitions.

8. The assay module of claim 7, wherein there are at least ten first and second fluid channels and ten first and second partitions.

9. The assay module of claim 7, further comprising third fluid channels connecting the second partitions to third partitions, wherein detection and imaging is performed in the third partitions.

10. The assay module of claim 7, wherein the first partitions and/or the second partitions provide mixing of the sample and the RNP1s, RNP2s, blocked nucleic acid molecules and/or reporter moieties via bubbling, ultrasonic perturbation, magnetic beads or push/pull pressure changes.

11. A method for performing an assay cascade on a sample comprising the steps of:
providing the assay module of claim 1, wherein the detection and imaging zones are in each of the first partitions;
providing the sample, RNP2s, blocked nucleic acid molecules, and reporter moieties to the sample splitting region;
using the pump to flow the sample, RNP2s, blocked nucleic acid molecules, and reporter moieties from the sample splitting region through the first fluid channels into the first partitions; and
detecting and imaging signals from the reporter moieties in the first partitions.

12. An assay module for identifying one or more target nucleic acids of interest from two or more source organisms in a sample comprising:
a bulk sample introduction region comprising at least one inlet and a fluid channel fluidically coupled sequentially to a plurality of first partitions;
a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, wherein different RNP1s reside in different partitions, wherein the different RNP1s are coupled to the partitions or to supports within the partitions, and wherein reacted reporter moieties, if present, can be detected and imaged in each of the partitions;
second ribonucleoprotein complexes (RNP2s) comprising a second nucleic acid-guided nuclease and a second guide nucleic acid complementary to a portion of blocked nucleic acid molecules;
the blocked nucleic acid molecules;
reporter moieties; and
a pump configured to provide negative or positive pressure to the assay module.

13. The assay module of claim 12, wherein the RNP1s are lyophilized.

14. The assay module of claim 12, wherein the RNP1s are air dried.

15. The assay module of claim 12, wherein the reporter moieties and blocked nucleic acid molecules are introduced in the bulk sample introduction region with the sample and the RNP2s are coupled to the first partitions or to supports within the partitions.

16. The assay module of claim 12, wherein the RNP2s, reporter moieties and blocked nucleic acid molecules are introduced in the bulk sample introduction region with the sample.

17. The assay module of claim 12, wherein the first partitions provide mixing of the sample and the reaction components.

18. A method for performing an assay cascade on a sample comprising the steps of:
providing the assay module of step 12;
providing the sample, RNP2s, blocked nucleic acid molecules, and reporter moieties to the bulk sample introduction region;
using the pump to flow the sample, RNP2s, blocked nucleic acid molecules, and reporter moieties from bulk sample introduction region through the fluid channel into each of the partitions; and
detecting and imaging signals from the reporter moieties in the partitions.

19. The method of claim 18, wherein the second providing step is performed at below room temperature.

20. An assay module for identifying one or more target nucleic acids of interest from two or more source organisms in a sample using reaction components comprising:
a bulk sample introduction region comprising at least one inlet and a fluid channel fluidically coupled to a plurality of first partitions;
a plurality of first ribonucleoprotein complexes (RNP1s) wherein the RNP1s comprise a first nucleic acid-guided nuclease and different first guide nucleic acids (gRNA1s) complementary to one or more loci in each genome of the two or more source organisms, wherein different RNP1s reside in different first partitions, and wherein the different RNP1s are coupled to the first partitions or to supports within the first partitions;
a plurality of second ribonucleoprotein complexes (RNP2s) comprising a second nucleic acid-guided nuclease and a second guide nucleic acid complementary to a portion of blocked nucleic acid molecules wherein the RNP2s reside in a plurality of second partitions fluidically coupled to the plurality of first partitions but separated from the plurality of first partitions by a frangible membrane, wherein the frangible membrane is ruptured at pressure X;
blocked nucleic acid molecules and reporter moieties;
a pump configured to provide negative or positive pressure to the assay module; and
detection and imaging zones adjacent to the second partitions.

21. The assay module of claim 20, wherein the RNP1s and RNP2s are lyophilized.

22. The assay module of claim 20, wherein the RNP1s and RNP2s are air dried.

23. The assay module of claim 20, wherein the reporter moieties and blocked nucleic acid molecules are introduced in the bulk sample introduction region with the sample.

24. The assay module of claim 20, wherein the reporter moieties and blocked nucleic acid molecules reside in the second partitions with the RNP2s.

25. The assay module of claim 20, wherein the reporter moieties and blocked nucleic acid molecules reside in the first partitions with the RNP1s.

26. A method for performing an assay cascade on a sample comprising the steps of:
providing the assay module of step 20;
providing the sample, blocked nucleic acid molecules, and reporter moieties to the bulk sample introduction region;
using the pump at a pressure less than X to flow the sample, blocked nucleic acid molecules, and reporter moieties from the bulk sample introduction region through the fluid channel into each of the first partitions;
allowing the sample to react with the RNP1s;
using the pump to provide a pressure X to rupture the frangible membrane between the first and second partitions;
allowing the unblocked nucleic acid molecules, if present, to react with the RNP2s in the second partitions; and
detecting and imaging signals from the reporter moieties in the second partitions.
